(19) United States
(12) Patent Application Publication
(10) Pub. No.: US 2025/0221410 A1
(43) Pub. Date: Jul. 10, 2025

(54) SUBSTITUTED 2-C-AZINES AND SALTS THEREOF, AND USE THEREOF AS HERBICIDAL ACTIVE SUBSTANCES

(71) Applicant: BAYER AKTIENGESELLSCHAFT, LEVERKUSEN (DE)

(72) Inventors: Ralf BRAUN, Leverkusen (DE); Ines HEINEMANN, Leverkusen (DE); Mohan PADMANABAN, Leverkusen (DE); Michael Peter BADART, Leverkusen (DE); Birgit BOLLENBACH-WAHL, Leverkusen (DE); Dirk SCHMUTZLER, Leverkusen (DE); Anna Maria REINGRUBER, Leverkusen (DE)

(21) Appl. No.: 18/849,823

(22) PCT Filed: Mar. 23, 2023
(86) PCT No.: PCT/EP2023/057451

§ 371 Date: Sep. 23, 2024

(30) Foreign Application Priority Data
Mar. 28, 2022 (EP) 22164820.7

(51) Int. Cl. A01N 43/54; A01N 41/06; A01N 43/42; A01N 43/56; A01N 43/80; A01N 43/82; A01P 13/00; C07D 239/30

(52) U.S. Cl. CPC A01N 43/54; A01N 41/06; A01N 43/42; A01N 43/56; A01N 43/80; A01N 43/82; A01P 13/00; C07D 239/30

(57) ABSTRACT

Substituted 2-C-azines of the general formula (I) are described,

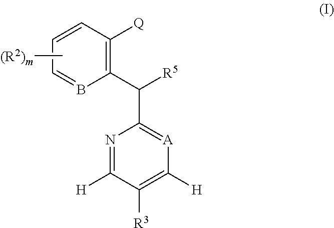

and the use thereof as herbicides, in particular for controlling broad-leaved weeds and/or weed grasses in crops of useful plants and/or as plant growth regulators for influencing the growth of crops of useful plants.

The present invention further relates to herbicidal and/or plant growth-regulating compositions comprising one or more compounds of the general formula (I).

The invention relates to the technical field of crop protection products, in particular that of herbicides for selective control of broad-leaved weeds and weed grasses in crops of useful plants.

Specifically, the present invention relates to substituted 2-C-azines and salts thereof, to processes for their preparation and to their use as herbicides.

In their application, crop protection products known to date for the selective control of harmful plants in crops of useful plants or active ingredients for controlling unwanted vegetation sometimes have disadvantages, whether (a) that they have insufficient herbicidal activity, if any, against particular harmful plants, (b) that the spectrum of harmful plants which can be controlled with an active ingredient is not wide enough, (c) that their selectivity in crops of useful plants is too low and/or (d) that they have a toxicologically unfavourable profile. Furthermore, some active ingredients which can be used as plant growth regulators for a number of useful plants cause undesirably reduced harvest yields in other useful plants or are compatible with the crop plant only within a narrow application rate range, if at all. Some of the known active ingredients cannot be produced economically on an industrial scale owing to precursors and reagents which are difficult to obtain, or they have only insufficient chemical stabilities.

In the case of other active ingredients, the activity is too highly dependent on environmental conditions, such as weather and soil conditions.

The herbicidal action of these known compounds, especially at low application rates, and/or the compatibility thereof with crop plants is still in need of improvement.

AU535637, EP8192, EP61913, JP61236766, WO2016/196606, WO2021/204706, GB2594931 and WO2016/010731 are among the documents that describe heteroaryloxybenzenes to which herbicidal action has been ascribed. WO2020/002089 and WO2022/002838 describe heteroaryloxypyridines to which herbicidal action has been ascribed. WO2020/193474 describes 2-heteroarylaminobenzenes to which herbicidal action has been ascribed.

By contrast, substituted 2-C-azines or salts thereof are yet to be described as active herbicidal ingredients.

Surprisingly, it has now been found that particular substituted 2-C-azines or salts thereof are particularly suitable as active herbicidal ingredients.

The present invention thus provides substituted 2-C-azines of the general formula (I) or salts thereof:
in which

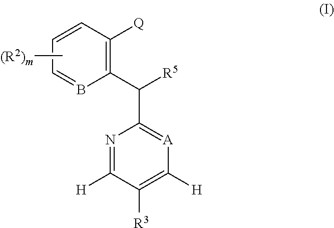

A is nitrogen or CR¹,
R¹ is hydrogen, CN or halogen,
B is CH, CR² or N,
Q is Y—(C₂-C₆)-haloalkyl, where Y denotes direct bond, oxygen, S(O)ₙ, CO or OSO₂, or
Q is Z-aryl or Z-heteroaryl, where aryl is substituted by 1 to 5 substituents independently selected from the group of R⁴, where heteroaryl is substituted by up to 2 substituents independently selected from the group of R⁴, and where Z denotes direct bond, O, S(O)ₙ or CH₂,
R² is independently halogen, cyano, nitro, amino, (C₁-C₄)-alkyl, (C₁-C₄)-haloalkyl, cyclopropyl, (C₁-C₄)-alkoxy, (C₁-C₄)-haloalkoxy or (C₁-C₄)-alkyl-S(O)ₙ—,
m is 0, 1, 2 or 3,
n is 0, 1 or 2,
R³ is hydrogen, halogen, cyano, nitro, (C₁-C₄)-alkyl, (C₁-C₄)-haloalkyl, (C₁-C₄)-alkoxy, (C₁-C₂)-haloalkoxy or (C₁-C₄)-alkyl-S(O)ₙ—, under the condition that, when A is CR¹, R³ must not be hydrogen,
R⁴ is halogen, cyano, nitro, (C₁-C₄)-alkyl, (C₁-C₅)-haloalkyl, (C₁-C₄)-alkoxy, (C₁-C₂)-haloalkoxy, (C₁-C₄)-alkoxymethyl, (C₁-C₄)-alkyl-S(O)ₙ—,
and
R⁵ is hydrogen or CN.

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. These salts then contain the conjugate base of the acid as anion. Suitable substituents in deprotonated form, for example sulfonic acids, particular sulfonamides or carboxylic acids, are capable of forming internal salts with groups, such as amino groups, which are themselves protonatable. Salts may also be formed by action of a base on compounds of the general formula (I). Suitable bases are, for example, organic amines such as trialkylamines, morpholine, piperidine and pyridine, and the hydroxides, carbonates and hydrogencarbonates of ammonium, alkali metals or alkaline earth metals, especially sodium hydroxide, potassium hydroxide, sodium carbonate, potassium carbonate, sodium hydrogencarbonate and potassium hydrogencarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{c}R^{d}]⁺ in which R^{a} to R^{d}are each independently an organic radical, especially alkyl, aryl, arylalkyl or alkylaryl. Also useful are alkylsulfonium and alkylsulfoxonium salts, such as (C₁-C₄)-trialkylsulfonium and (C₁-C₄)-trialkylsulfoxonium salts.

The inventive substituted 2-C-azines of the general formula (I), depending on external conditions such as pH, solvent and temperature, may possibly be present in various tautomeric structures, all of which are embraced by the general formula (I).

The compounds of the formula (I) used in accordance with the invention and salts thereof are referred to hereinafter as “compounds of the general formula (I)”.

The invention more preferably provides compounds of the general formula (I) in which
A is nitrogen or CR¹,
R¹ is hydrogen, CN or halogen,
B is CH, CR² or N,
Q is Y—(C₃-C₅)-haloalkyl, where Y denotes direct bond, oxygen, S(O)ₙ, CO or OSO₂, or
Q is Z-aryl or Z-heteroaryl, where aryl is substituted by 1 to 3 substituents independently selected from the group of R⁴, where heteroaryl is substituted by up to 2 substituents independently selected from the group of R⁴, and where Z denotes direct bond, O, S(O)ₙ or CH₂,
R² is independently halogen, cyano, nitro, amino, (C₁-C₄)-alkyl, (C₁-C₄)-haloalkyl, cyclopropyl, (C₁-C₄)-alkoxy, (C₁-C₄)-haloalkoxy or (C₁-C₄)-alkyl-S(O)ₙ—,
m is 0, 1, 2 or 3,
n is 0, 1 or 2,
R³ is hydrogen, halogen, cyano, (C₁-C₄)-alkyl, (C₁-C₄)-haloalkyl, (C₁-C₄)-alkoxy or (C₁-C₂)-haloalkoxy,
   under the condition that, when A is CR¹, R³ must not be hydrogen,
R⁴ is halogen, cyano, (C₁-C₄)-alkyl, (C₁-C₈)-haloalkyl, (C₁-C₄)-alkoxymethyl,
and
R⁵ is hydrogen or CN.

The invention very particularly preferably provides compounds of the general formula (I) in which
A is nitrogen or CR¹,
R¹ is hydrogen, cyano, fluorine or chlorine,
B is CH, CR² or N,
Q is Y—(C₃-C₅)-haloalkyl, where Y denotes direct bond, oxygen, S, CO or OSO₂, or
Q is Z-aryl or Z-heteroaryl, where aryl is substituted by 1 or 2 substituents independently selected from the group of R⁴, where heteroaryl is substituted by up to 2 substituents independently selected from the group of R⁴, and where Z denotes direct bond, O, S or CH₂,
R² is independently fluorine, chlorine, bromine, cyano, methyl, CF₃ or methoxy,
m is 0, 1 or 2,
R³ is hydrogen, chlorine, bromine, fluorine, cyano, methyl, CF₃, methoxy or CHF₂O, under the condition that, when A is CR¹, R³ must not be hydrogen,
R⁴ is fluorine, chlorine, methyl, CHF₂, CF₃ or methoxymethyl,
and
R⁵ is hydrogen or CN.

The invention likewise further preferably provides compounds of the general formula (I), in which
A is nitrogen or CR¹,
R¹ is hydrogen, cyano, fluorine or chlorine,
B is CH, CR² or N,
Q is Y—(C₃-C₅)-haloalkyl, where Y denotes direct bond, oxygen, S, CO or OSO₂, or
Q is Z-aryl or Z-heteroaryl, where aryl is substituted by 1 or 2 substituents independently selected from the group of R⁴, where heteroaryl is substituted by up to 2 substituents independently selected from the group of R⁴, and where Z denotes direct bond, O, S or CH₂,
R² is independently fluorine, chlorine, bromine, cyano, methyl, CF₃ or methoxy,
m is 0, 1 or 2,
R³ is hydrogen, chlorine, bromine, fluorine, cyano, methyl, CF₃, methoxy or CHF₂O, under the condition that, when A is CR¹, R³ must not be hydrogen,
R⁴ is fluorine, chlorine, methyl, CHF₂, CF₃ or methoxymethyl,
and
R⁵ is hydrogen or CN.

The invention likewise further preferably provides compounds of the general formula (I), in which
A is nitrogen, CH, CCN or CF,
B is CH,
Q is (CH₂)₃CF₃, (CH₂)₄CF₃, S(CH₂)₃CF₃, S(CH₂)₃Cl, SO(CH₂)₃Cl, SO(CH₂)₂CF₃, SO(CH₂)₃CF₃, SO₂(CH₂)₃CF₃, O(CH₂)₃CF₃, C(O)(CH₂)₃CF₃, OSO₂(CH₂)₂CF₃, OSO₂(CH₂)₃CF₃, OSO₂(CH₂)₃Cl, 4-fluorobenzyl, 4-chlorophenyl, 3,4-difluorophenyl, 4-fluorophenoxy, 5-Cl-pyrimidin-2-oxy, 5-F-pyrimidin-2-oxy, 5-Cl-3-F-pyridin-2-oxy, 4-fluorophenylsulfanyl, 2,4-Cl₂-phenylsulfonyl, 2,4-Cl₂-phenylsulfanyl, 2,4-Cl₂-phenylsulfinyl, 3-(CHF₂)-isoxazol-5-yl, 5-(CHF₂)-isoxazol-3-yl, 1,2,4-oxadiazol-3-yl, 5-methyl-1,2,4-oxadiazol-3-yl, 5-(CHF₂)-1,2,4-oxadiazol-3-yl, 5-(CF₃)-1,2,4-oxadiazol-3-yl, 5-Cl-pyrimidin-2-ylsulfanyl, 5-Cl-pyrimidin-2-ylsulfinyl, 5-Cl-pyrimidin-2-ylsulfonyl, 5-F-pyrimidin-2-ylsulfanyl, 4-(CF₃)-pyrazol-1-yl, 4-Cl-pyrazol-1-ylmethyl, 4-Br-pyrazol-1-ylmethyl or 4-(CF₃)-pyrazol-1-ylmethyl,
R² is independently fluorine, chlorine, bromine, cyano, methyl, CF₃ or methoxy,
m is 0, 1 or 2,
R³ is chlorine, bromine or fluorine
and
R⁵ is hydrogen or CN.

The invention more preferably provides compounds of the general formula (I), in which
A is nitrogen, CH or CF,
B is CH,
Q is (CH₂)₃CF₃, S(CH₂)₃CF₃, SO(CH₂)₃CF₃, O(CH₂)₃CF₃, OSO₂(CH₂)₃CF₃, OSO₂(CH₂)₃Cl, 4-fluorobenzyl, 3,4-difluorophenyl, 4-fluorophenoxy, 4-fluorophenylsulfanyl, 2,4-Cl₂-phenylsulfanyl, 4-(CF₃)-pyrazol-1-yl, 5-Cl-pyrimidin-2-oxy, 5-F-pyrimidin-2-oxy, 5-Cl-3-F-pyridin-2-oxy, 3-(CHF₂)-isoxazol-5-yl or 5-methyl-1,2,4-oxadiazol-3-yl,
R² is independently fluorine, chlorine, bromine, cyano, methyl, CF₃ or methoxy,
m is 0, 1 or 2,
R³ is chlorine, bromine or fluorine
and
R⁵ is hydrogen or CN.

The definitions of radicals listed above in general terms or within areas of preference apply both to the end products of the general formula (I) and correspondingly to the starting materials or intermediates required for preparation in each case. These radical definitions can be combined with one another as desired, i.e. including combinations between the given preferred ranges.

Of particular interest, primarily for reasons of higher herbicidal activity, better selectivity and/or better preparability, are inventive compounds of the general formula (I) given or salts thereof or the inventive use thereof in which individual radicals have one of the preferred meanings already specified or specified below, or in particular those in which one or more of the preferred meanings already specified or specified below occur in combination.

With regard to the compounds of the invention, the terms used above and further down will be elucidated. These are familiar to the person skilled in the art and especially have the definitions elucidated hereinafter:

Unless defined differently, names of chemical groups should generally be understood such that attachment to the skeleton or the remainder of the molecule is via the structural element of the relevant chemical group mentioned last, i.e. for example in the case of (C₁-C₄)-alkoxy via the oxygen atom, in the case of (C₁-C₄)-alkyl-S(O)ₙ— via the sulfur atom, and in the case of (C₁-C₄)-alkoxymethyl via the carbon atom of the methyl group.

The term “halogen” denotes, for example, fluorine, chlorine, bromine or iodine. If the term is used for a radical, “halogen” denotes, for example, a fluorine, chlorine, bromine or iodine atom.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₂—C)-alkyl”, correspondingly also encompass straight-chain or branched alkyl radicals with a greater number of carbon atoms, i.e. according to the example also the alkyl radicals having 5 and 6 carbon atoms.

“Haloalkyl” denotes alkyl partly or fully substituted by identical or different halogen atoms, e.g. monohaloalkyl, for example CH₂CH₂Cl, CH₂CH₂Br, CHClCH₃, CH₂Cl, CH₂F, CH₂CH₂CF₃; perhaloalkyl, for example CCl₃, CClF₂, CFCl₂, CF₂CClF₂, CF₂CClFCF₃; polyhaloalkyl, for example CH₂CHFCl, CF₂CClFH, CF₂CBrFH, CH₂CF₃; the term perhaloalkyl also encompasses the term perfluoroalkyl.

“Alkoxy” denotes an alkyl radical bonded via an oxygen atom, for example (but not limited to) (C₁-C₄)-alkoxy such as methoxy, ethoxy, propoxy, 1-methylethoxy, butoxy, 1-methylpropoxy, 2-methylpropoxy and 1,1-dimethylethoxy.

Haloalkoxy is, for example, OCF₃, OCHF₂, OCH₂F, OCF₂CF₃, OCH₂CF₃ and OCH₂CH₂C₁.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

When a base structure is substituted “by one or more radicals” from a list of radicals (=group) or a generically defined group of radicals, this in each case includes simultaneous substitution by a plurality of identical and/or structurally different radicals.

According to the invention, the expression “heteroaryl” represents heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups of the invention may also be substituted by one or more identical or different radicals. If two adjacent carbon atoms are part of a further aromatic ring, the systems are fused heteroaromatic systems, such as benzofused or polyannelated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

If the compounds can form, through a hydrogen shift, tautomers whose structure would not formally be covered by the general formula (I), these tautomers are nevertheless encompassed by the definition of the inventive compounds of the general formula (I), unless a particular tautomer is under consideration.

For example, many carbonyl compounds may be present both in the keto form and in the enol form, both forms being encompassed by the definition of the compound of the general formula (I).

Depending on the nature of the substituents and the manner in which they are attached, the compounds of the general formula (I) may be present as stereoisomers. The possible stereoisomers defined by the specific three-dimensional form thereof, such as enantiomers, diastereomers, Z and E isomers, are all encompassed by the general formula (I). If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

If the compounds are obtained as solids, the purification can also be carried out by recrystallisation or digestion. If individual compounds of the general formula (I) cannot be obtained in a satisfactory manner by the routes described below, they can be prepared by derivatisation of other compounds of the general formula (I).

Suitable isolation methods, purification methods and methods for separating stereoisomers of compounds of the general formula (I) are methods generally known to the person skilled in the art from analogous cases, for example by physical processes such as crystallisation, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallisation, for example of diastereomeric salts which can be obtained from the diastereomer mixtures using optically active acids and, if appropriate, provided that acidic groups are present, using optically active bases.

The present invention also claims processes for preparing the inventive compounds of the general formula (I).

The inventive compounds of the general formula (I) can be prepared, inter alia, using known processes. The synthesis routes used and examined proceed from commercially available or easily preparable building blocks. In the schemes which follow, the moieties A, B, Q, R², R³ and m of the general formula (I) have the meanings defined above, unless illustrated but non-limiting definitions are given.

Inventive compounds can be prepared, for example, by the method specified in Scheme 1 below.

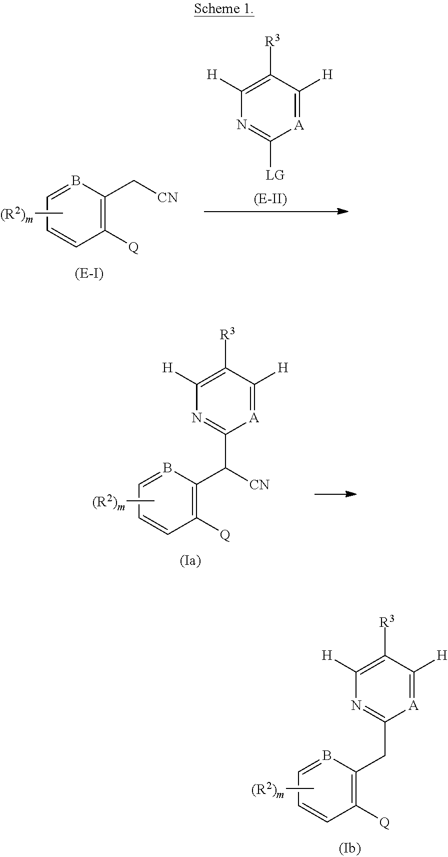

The pyri(mi)dines of the general formula (Ia) can be prepared by coupling the corresponding acetonitriles (E-I) with the pyri(mi)dines (EII), where LG is a leaving group, in the presence of a base for example. The base required for this purpose may, for example, be a carbonate salt of an alkali metal (for example sodium or potassium) or a hydride of an alkali metal (for example sodium). The reactions are generally conducted in an organic solvent, for example dioxane, dimethyl sulfoxide or dimethylformamide, at temperatures between 0° C. and the boiling point of the solvent.

The pyri(mi)dines of the general formula (Ib) can be prepared, for example, via a decarboxylating hydrolysis of the corresponding acetonitriles (Ia). Corresponding conditions are, for example, aqueous acids (for example hydrochloric acid) with heating up to the boiling point.

The acetonitriles of the general formula (E-I) are known from the literature and can be prepared, for example, by methods described in Helvetica Chimica Acta (2003), 86(2), 343-360, in Angewandte Chemie, International Edition (2011), 50(19), 4470-4474, and similar methods.

Selected detailed synthesis examples for the inventive compounds of the general formula (I) are adduced below. The ¹H NMR, ¹³C NMR and ¹⁹F NMR spectroscopy data reported for the chemical examples described in the sections which follow (400 MHz for ¹H NMR and 150 MHz for ¹³C NMR and 375 MHz for ¹⁹F NMR, solvent CDCl₃, CD₃OD or d₆-DMSO, internal standard: tetramethylsilane δ=0.00 ppm) were obtained on a Bruker instrument, and the signals listed have the meanings given below: b=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, what is reported is either the significant signals for each of the two diastereomers or the characteristic signal of the main diastereomer. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### SYNTHESIS EXAMPLES

### Table Example Number 1-34: (5-Chloropyrimidin-2-yl)[3-fluoro-2-(4-fluorophenoxy)phenyl]acetonitrile

### Synthesis Stage 1: 3-Fluoro-2-(4-fluorophenoxy)benzaldehyde

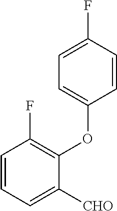 [SMILES: O=Cc1cccc(F)c1Oc1ccc(F)cc1]

A solution of 2.0 g of 2,3-difluorobenzaldehyde (14.1 mmol, 1.0 eq), 2.37 g of 2-fluorophenol (21.1 mmol, 1.5 eq) and 3.89 g of potassium carbonate (28.1 mmol, 2.0 eq) in 30 ml of DMF was stirred at 100° C. for 3 h. This was followed by dilution with 50 ml of ether, washing with 2N sodium hydroxide solution (30 ml×2) and saturated sodium chloride solution, drying over sodium sulfate and concentration under reduced pressure. The residue was purified by column chromatography. The yield was 2.82 g (85%). ¹H-NMR (400.0 MHz, CDCl₃): 10.35 (s, 1H); 7.75 (m, 1H); 7.43 (m, 1H); 7.32 (m, 1H); 7.01 (m, 2H); 6.91 (m, 2H).

### Synthesis Stage 2: 3-Fluoro-2-(4-fluorophenoxy)benzyl alcohol

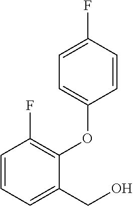 [SMILES: OCc1cccc(F)c1Oc1ccc(F)cc1]

To 2.8 g of 3-fluoro-2-(4-fluorophenoxy)benzaldehyde (12 mmol, 1.0 eq) in a mixture of 44 ml of THF and 22 ml of MeOH was added 0.91 g of sodium borohydride (24 mmol, 2.0 eq), and the mixture was stirred at room temperature for 3 h. This was followed by addition of water to the solution and extraction with ethyl acetate. The organic phase was concentrated under reduced pressure, and the residue was taken up with dichloromethane, dried over sodium sulfate and concentrated under reduced pressure. The product obtained was used without further purification. The yield was 2.77 g (98% crude). ¹H-NMR (400.0 MHz, CDCl₃): 7.30 (m, 1H); 7.23 (m, 1H); 7.13 (m, 1H); 6.97 (m, 2H); 6.84 (m, 2H); 4.69 (s, 2H); 1.79 (bs, 1H)

### Synthesis Stage 3: 3-Fluoro-2-(4-fluorophenoxy)benzyl chloride

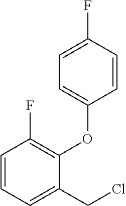 [SMILES: Fc1ccc(Oc2c(F)cccc2CCl)cc1]

To a solution of 2.70 g of 3-fluoro-2-(4-fluorophenoxy)benzyl alcohol (11.4 mmol, 1.0 eq) in 18 ml of toluene was added 1.0 ml of thionyl chloride, and the mixture was stirred at room temperature for 5 h. This was followed by addition to water and extraction with dichloromethane. The organic phase was dried with sodium sulfate and concentrated under reduced pressure. The product obtained was used without further purification. The yield was 3.29 g (100% crude). ¹H-NMR (400.0 MHz, CDCl₃): 7.31-7.12 (m, 3H); 6.98 (m, 2H); 6.87 (m, 2H); 4.60 (s, 2H)

### Synthesis Stage 4: (3-Fluoro-2-(4-fluorophenoxy)phenyl)acetonitrile

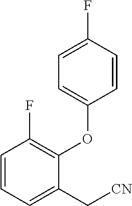 [SMILES: N#CCc1cccc(F)c1Oc1ccc(F)cc1]

3.2 g of 3-fluoro-2-(4-fluorophenoxy)benzyl chloride (12.6 mmol, 1.0 eq) and 982 mg of potassium cyanide (15.1 mmol, 1.2 eq) was heated under reflux in a mixture of 5 ml of water and 25 ml of ethanol for 6 h. This was followed by concentration and taking-up of the residue with dichloromethane. The organic phase was dried with sodium sulfate and concentrated under reduced pressure, and the residue was purified by column chromatography. The yield was 1.46 g (47%). ¹H-NMR (400.0 MHz, CDCl₃): 7.33 (m, 1H); 7.26 (m, 1H); 7.18 (m, 1H); 6.99 (m, 2H); 6.83 (m, 2H); 3.72 (s, 2H);

### Synthesis Stage 5: (5-Chloropyrimidin-2-yl)[3-fluoro-2-(4-fluorophenoxy)phenyl]acetonitrile

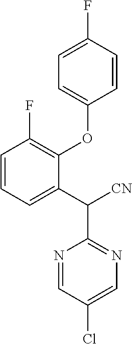 [SMILES: N#CC(c1ncc(Cl)cn1)c1cccc(F)c1Oc1ccc(F)cc1]

To 500 mg of (3-fluoro-2-(4-fluorophenoxy)phenyl)acetonitrile (2.0 mmol, 1.0 eq) in 15 ml of THF was added 179 mg of sodium hydride (60%) (4.49 mmol, 2.2 eq), and the mixture was stirred at room temperature for 30 min. Subsequently, 365 mg of 2,5-dichloropyrimidine (2.45 mmol, 1.2 eq) was added, and the mixture was stirred at room temperature overnight. Thereafter, the mixture was admixed with 2N hydrochloric acid and extracted with dichloromethane. The organic phase was dried with sodium sulfate and concentrated under reduced pressure, and the residue was purified by column chromatography. The yield was 631 mg (87%).

### Table Example Number 1-35: 5-Chloro-2-[3-fluoro-2-(4-fluorophenoxy)benzyl]pyrimidine

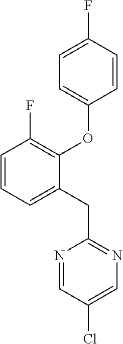 [SMILES: Fc1ccc(Oc2c(F)cccc2Cc2ncc(Cl)cn2)cc1]

530 mg of (5-chloropyrimidin-2-yl)[3-fluoro-2-(4-fluorophenoxy)phenyl]acetonitrile (1.48 mmol) in a mixture of 6 ml of glacial acetic acid and 2 ml of conc. hydrochloric acid was stirred at 100° C. for 8 h. Subsequently, the mixture was concentrated and the residue was purified by column chromatography. The yield was 200 mg (41%).

In analogy to the preparation examples cited above and recited at the appropriate point, the compounds of the general formula specified hereinafter and shown in Table 1 are obtained.

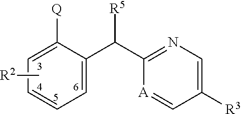

TABLE 1

| Example | | | | | |
|---|---|---|---|---|---|
| number | R² | R³ | R⁵ | A | Q |
| 1-1 | H | Cl | H | N | (CH₂)₃CF₃ |
| 1-2 | H | Cl | H | N | CH₂CF₄ |
| 1-3 | H | F | H | N | (CH₂)₃CF₃ |
| 1-4 | H | Cl | H | N | S(CH₂)₃CF₃ |
| 1-5 | H | Cl | H | N | SO(CH₂)₃CF₃ |
| 1-6 | H | Cl | H | N | SO₂(CH₂)₃CF₃ |
| 1-7 | H | F | H | N | S(CH₂)₃CF₃ |
| 1-8 | H | Cl | H | N | O(CH₂)₃CF₃ |
| 1-9 | H | Cl | H | N | C(O)(CH₂)₃CF₃ |
| 1-10 | H | Cl | H | N | OSO₂(CH₂)₃CF₃ |
| 1-11 | H | F | H | N | OSO₂(CH₂)₃CF₃ |
| 1-12 | H | Cl | H | N | OSO₂(CH₂)₂CF₃ |
| 1-13 | H | F | H | N | OSO₂(CH₂)₂CF₃ |
| 1-14 | 3-F | Cl | H | N | OSO₂(CH₂)₃CF₃ |
| 1-15 | 3-F | F | H | N | OSO₂(CH₂)₃CF₃ |
| 1-16 | 3-F | Cl | H | N | OSO₂(CH₂)₂CF₃ |
| 1-17 | 3-F | F | H | N | OSO₂(CH₂)₂CF₃ |
| 1-18 | 3-Cl | Cl | H | N | OSO₂(CH₂)₃CF₃ |
| 1-19 | 3-Cl | F | H | N | OSO₂(CH₂)₃CF₃ |
| 1-20 | 3-Cl | Cl | H | N | OSO₂(CH₂)₂CF₃ |
| 1-21 | 3-Cl | F | H | N | OSO₂(CH₂)₂CF₃ |
| 1-22 | 3-F | Cl | H | N | OSO₂(CH₂)₃C₂F₅ |
| 1-23 | H | Cl | H | N | 4-fluorobenzyl |
| 1-24 | H | Cl | H | N | 4-fluorophenyl |
| 1-25 | H | Cl | H | N | 4-chlorophenyl |
| 1-26 | H | Cl | H | N | 3,4-difluorophenyl |
| 1-27 | H | F | H | N | 3,4-difluorophenyl |
| 1-28 | H | Cl | CN | N | 4-fluorophenoxy |
| 1-29 | H | Cl | H | N | 4-fluorophenoxy |
| 1-30 | 3-Me | Cl | CN | N | 4-fluorophenoxy |
| 1-31 | 3-Me | Cl | H | N | 4-fluorophenoxy |
| 1-32 | 3-MeO | Cl | CN | N | 4-fluorophenoxy |
| 1-33 | 3-MeO | Cl | H | N | 4-fluorophenoxy |
| 1-34 | 3-F | Cl | CN | N | 4-fluorophenoxy |
| 1-35 | 3-F | Cl | H | N | 4-fluorophenoxy |
| 1-36 | 3-Br | Cl | CN | N | 4-fluorophenoxy |
| 1-37 | 3-Br | Cl | H | N | 4-fluorophenoxy |
| 1-38 | 3-CN | Cl | H | N | 4-fluorophenoxy |
| 1-39 | 3-CF₃ | Cl | CN | N | 4-fluorophenoxy |
| 1-40 | 3-CF₃ | Cl | H | N | 4-fluorophenoxy |
| 1-41 | H | Cl | CN | N | 4-(CF₃)-pyrazol-1-yl |
| 1-42 | H | Cl | H | N | 4-(CF₃)-pyrazol-1-yl |
| 1-43 | 3-Br | Cl | CN | N | 4-(CF₃)-pyrazol-1-yl |
| 1-44 | 3-Br | Cl | H | N | 4-(CF₃)-pyrazol-1-yl |
| 1-45 | 3-CN | Cl | H | N | 4-(CF₃)-pyrazol-1-yl |
| 1-46 | 3-F | Cl | CN | N | 4-(CF₃)-pyrazol-1-yl |
| 1-47 | 3-F | Cl | H | N | 4-(CF₃)-pyrazol-1-yl |
| 1-48 | 3-Me | Cl | CN | N | 4-(CF₃)-pyrazol-1-yl |
| 1-49 | 3-Me | Cl | H | N | 4-(CF₃)-pyrazol-1-yl |
| 1-50 | H | Cl | H | N | 5-Cl-pyrimidin-2-oxy |
| 1-51 | 6-F | Cl | H | N | 5-Cl-pyrimidin-2-oxy |
| 1-52 | 6-F | Cl | H | N | 5-F-pyrimidin-2-oxy |
| 1-53 | 6-Br | Cl | H | N | 5-Cl-pyrimidin-2-oxy |
| 1-54 | 6-Br | Cl | H | N | 5-F-pyrimidin-2-oxy |
| 1-55 | 6-Br | Cl | H | N | 5-Cl-3-F-pyrimidin-2-oxy |
| 1-56 | 6-Cl | Cl | H | N | 5-Cl-pyrimidin-2-oxy |
| 1-57 | 6-Cl | Cl | H | N | 5-F-pyrimidin-2-oxy |
| 1-58 | 6-Cl | Cl | H | N | 5-Cl-3-F-pyrimidin-2-oxy |
| 1-59 | 6-CN | Cl | H | N | 5-F-pyrimidin-2-oxy |
| 1-60 | 6-F | Cl | H | N | O(CH₂)₃CF₃ |
| 1-61 | 6-CN | Cl | H | N | 5-Cl-pyrimidin-2-oxy |
| 1-62 | 6-CN | F | H | N | 5-Cl-pyrimidin-2-oxy |
| 1-63 | 6-CN | F | H | N | 5-F-pyrimidin-2-oxy |
| 1-64 | 6-Me | Cl | H | N | 5-Cl-pyrimidin-2-oxy |
| 1-65 | 6-Me | Cl | H | N | 5-F-pyrimidin-2-oxy |
| 1-66 | 6-Me | Cl | H | N | 5-Cl-3-F-pyridin-2-oxy |
| 1-67 | H | F | H | N | 4-fluorophenoxy |
| 1-68 | H | Cl | CN | N | (CH₂)₃CF₃ |
| 1-69 | H | Cl | CN | N | 4-fluorobenzyl |
| 1-70 | H | F | H | N | 5-Cl-pyrimidin-2-oxy |
| 1-71 | 3-CN | Cl | CN | N | 4-(CF₃)-pyrazol-1-yl |
| 1-72 | H | F | CN | N | (CH₂)₃CF₃ |
| 1-73 | H | Cl | CN | CH | (CH₂)₃CF₃ |
| 1-74 | H | Cl | H | CH | 4-fluorophenoxy |
| 1-75 | H | H | H | N | 5-F-pyrimidin-2-oxy |
| 1-76 | H | H | H | N | 5-Cl-pyrimidin-2-oxy |
| 1-77 | H | H | H | N | 5-Cl-3-F-pyridin-2-oxy |
| 1-78 | H | Cl | CN | N | 3,4-difluorophenyl |
| 1-79 | H | F | CN | N | 3,4-difluorophenyl |
| 1-80 | H | Cl | CN | CH | 3,4-difluorophenyl |
| 1-81 | H | F | CN | N | 4-fluorobenzyl |
| 1-82 | H | Cl | CN | CH | 4-fluorobenzyl |
| 1-83 | H | Br | CN | N | 4-fluorophenoxy |
| 1-84 | H | Cl | CN | CH | 4-fluorophenoxy |
| 1-85 | H | Cl | CN | CCN | 4-fluorophenoxy |
| 1-86 | H | Cl | CN | N | 4-fluorophenylsulfanyl |
| 1-87 | H | Br | H | N | 4-fluorophenoxy |
| 1-88 | H | F | CN | N | 4-fluorophenylsulfanyl |
| 1-89 | H | F | H | N | 5-F-pyrimidin-2-oxy |
| 1-90 | H | F | H | N | 5-Cl-3-F-pyridin-2-oxy |
| 1-91 | H | Cl | H | CH | 5-Cl-pyrimidin-2-oxy |
| 1-92 | H | F | H | CH | 5-Cl-pyrimidin-2-oxy |
| 1-93 | H | F | H | CH | 5-F-pyrimidin-2-oxy |
| 1-94 | H | Cl | H | CH | (CH₂)₃CF₃ |
| 1-95 | H | F | H | N | 4-fluorobenzyl |
| 1-96 | H | Cl | H | CH | 4-fluorobenzyl |
| 1-97 | H | Cl | H | CH | 3,4-difluorophenyl |
| 1-98 | H | H | H | N | 4-fluorophenoxy |
| 1-99 | H | Cl | H | N | 4-fluorophenylsulfanyl |
| 1-100 | H | Cl | H | CH | 5-F-pyrimidin-2-oxy |
| 1-101 | H | Cl | H | CH | 5-Cl-3-F-pyridin-2-oxy |
| 1-102 | H | Cl | CN | CH | 4-fluorophenylsulfanyl |
| 1-103 | H | F | H | CH | 5-Cl-3-F-pyridin-2-oxy |
| 1-104 | H | F | H | N | 4-fluorophenylsulfanyl |
| 1-105 | H | Cl | H | CH | 4-fluorophenylsulfanyl |
| 1-106 | H | Cl | H | CF | 4-fluorophenylsulfanyl |
| 1-107 | H | Cl | CN | N | S(CH₂)₂CF₃ |
| 1-108 | H | F | CN | N | S(CH₂)₂CF₃ |
| 1-109 | H | Cl | CN | CH | S(CH₂)₂CF₃ |
| 1-110 | H | Cl | H | CF | S(CH₂)₂CF₃ |
| 1-111 | H | F | H | N | S(CH₂)₂CF₃ |
| 1-112 | H | Cl | H | N | S(CH₂)₂CF₃ |
| 1-113 | H | Cl | CN | CF | S(CH₂)₂CF₃ |
| 1-114 | H | Cl | CN | N | S(CH₂)₃CF₃ |
| 1-115 | H | F | CN | N | S(CH₂)₃CF₃ |
| 1-116 | H | Cl | CN | CF | S(CH₂)₃CF₃ |
| 1-117 | H | Cl | CN | CH | S(CH₂)₃CF₃ |
| 1-118 | H | Cl | CN | N | CH₂CF₂ |
| 1-119 | H | Cl | H | N | CH₂CF₂ |
| 1-120 | H | F | CN | N | CH₂CF₂ |
| 1-121 | H | F | H | N | CH₂CF₂ |
| 1-122 | H | Cl | CN | CH | CH₂CF₂ |
| 1-123 | H | Cl | H | CH | CH₂CF₂ |
| 1-124 | H | F | CN | N | 4-(CF₃)-pyrazol-1-yl |
| 1-125 | H | Cl | CN | CCN | 4-(CF₃)-pyrazol-1-yl |
| 1-126 | H | Cl | CN | CF | 4-(CF₃)-pyrazol-1-yl |
| 1-127 | H | Cl | H | CF | 3,4-difluorophenyl |
| 1-128 | 3-CN | F | CN | N | 4-(CF₃)-pyrazol-1-yl |
| 1-129 | H | F | H | N | 4-(CF₃)-pyrazol-1-yl |
| 1-130 | H | Cl | H | CF | 4-(CF₃)-pyrazol-1-yl |
| 1-131 | 3-CN | F | H | N | 4-(CF₃)-pyrazol-1-yl |
| 1-132 | H | Cl | CN | N | 3-(CHF₂)-isoxazol-5-yl |
| 1-133 | H | Cl | H | N | 3-(CHF₂)-isoxazol-5-yl |
| 1-134 | H | F | CN | N | 3-(CHF₂)-isoxazol-5-yl |
| 1-135 | H | F | H | N | 3-(CHF₂)-isoxazol-5-yl |
| 1-136 | H | Cl | CN | N | 2,4-Cl₂-phenylsulfanyl |
| 1-137 | H | Cl | H | N | 2,4-Cl₂-phenylsulfanyl |
| 1-138 | 3-Br | Cl | CN | N | 4-(CF₃)-pyrazol-1-yl |
| 1-139 | H | Cl | CN | N | 5-(CHF₂)-isoxazol-3-yl |
| 1-140 | 3-Cl | Cl | H | N | 3-(CHF₂)-isoxazol-5-yl |
| 1-141 | H | Cl | H | N | 4-fluorophenylsulfinyl |
| 1-142 | H | Cl | H | N | 4-fluorophenylsulfinyl |
| 1-143 | H | F | H | N | 4-fluorophenylsulfinyl |
| 1-144 | H | F | H | N | 4-fluorophenylsulfinyl |
| 1-145 | H | Cl | H | N | 2,4-Cl₂-phenylsulfinyl |
| 1-146 | H | F | CN | N | 2,4-Cl₂-phenylsulfinyl |
| 1-147 | H | F | H | N | 2,4-Cl₂-phenylsulfinyl |
| 1-148 | H | F | H | N | 2,4-Cl₂-phenylsulfinyl |
| 1-149 | H | F | H | N | SO(CH₂)₂CF₃ |
| 1-150 | H | F | H | N | SO₂(CH₂)₂CF₃ |
| 1-151 | 3-F | Cl | H | N | 5-Cl-pyrimidin-2-oxy |
| 1-152 | 3-F | Cl | H | N | 5-F-pyrimidin-2-oxy |
| 1-153 | 3-F | F | H | N | 5-F-pyrimidin-2-oxy |
| 1-154 | 3-F | F | H | N | 5-Cl-pyrimidin-2-oxy |
| 1-155 | 3-Cl | Cl | H | N | 5-F-pyrimidin-2-oxy |
| 1-156 | 3-Cl | F | H | N | 5-F-pyrimidin-2-oxy |
| 1-157 | 3-Cl | F | H | N | 5-Cl-pyrimidin-2-oxy |
| 1-158 | H | Cl | H | N | 1,2,4-oxadiazol-3-yl |
| 1-159 | H | Cl | H | N | 5-methyl-1,2,4-oxadiazol-3-yl |
| 1-160 | H | Cl | H | N | 5-(CHF₂)-1,2,4-oxadiazol-3-yl |
| 1-161 | H | Cl | H | N | 5-(CF₃)-1,2,4-oxadiazol-3-yl |
| 1-162 | 3-Me | F | H | N | 5-F-pyrimidin-2-oxy |
| 1-163 | H | F | H | N | 2,4-Cl₂-phenylsulfonyl |
| 1-164 | H | Cl | H | N | 2,4-Cl₂-phenylsulfonyl |
| 1-165 | 3-Me | Cl | H | N | 5-F-pyrimidin-2-oxy |
| 1-166 | 3-Me | Cl | H | N | 5-Cl-pyrimidin-2-oxy |
| 1-167 | H | Cl | H | N | 5-Cl-pyrimidin-2-ylsulfanyl |
| 1-168 | H | Cl | H | N | S(CH₂)₃CF₃ |
| 1-169 | H | Cl | H | N | 5-Cl-pyrimidin-2-ylsulfanyl |
| 1-170 | H | Cl | H | N | 5-Cl-pyrimidin-2-ylsulfonyl |
| 1-171 | H | Cl | H | N | 5-F-pyrimidin-2-oxy |
| 1-172 | H | F | CN | N | 4-Cl-pyrazol-1-ylmethyl |
| 1-173 | H | F | H | N | 4-Cl-pyrazol-1-ylmethyl |
| 1-174 | H | Cl | CN | N | 4-Cl-pyrazol-1-ylmethyl |
| 1-175 | H | Cl | H | N | 4-Cl-pyrazol-1-ylmethyl |
| 1-176 | H | F | CN | N | 4-Br-pyrazol-1-ylmethyl |
| 1-177 | H | F | H | N | 4-Br-pyrazol-1-ylmethyl |
| 1-178 | H | Cl | CN | N | 4-Br-pyrazol-1-ylmethyl |
| 1-179 | H | Cl | H | N | 4-Br-pyrazol-1-ylmethyl |
| 1-180 | H | F | CN | N | 4-(CF₃)-pyrazol-1-ylmethyl |
| 1-181 | H | F | H | N | 4-(CF₃)-pyrazol-1-ylmethyl |
| 1-182 | H | Cl | CN | N | 4-(CF₃)-pyrazol-1-ylmethyl |
| 1-183 | H | Cl | H | N | 4-(CF₃)-pyrazol-1-ylmethyl |
| 1-184 | 3-Me | Cl | H | N | OSO₂(CH₂)₃Cl |
| 1-185 | 3-F | F | H | N | OSO₂(CH₂)₃Cl |
| 1-186 | 3-F | Br | H | N | OSO₂(CH₂)₃CF₃ |
| 1-187 | 3-F | Br | H | N | OSO₂(CH₂)₂CF₃ |
| 1-188 | 3-Me | Cl | H | N | OSO₂(CH₂)₃CF₃ |
| 1-189 | 3-Me | Cl | H | N | OSO₂(CH₂)₂CF₃ |
| 1-190 | 3-Cl | Cl | H | N | OSO₂(CH₂)₃Cl |
| 1-191 | 3-Cl | F | H | N | OSO₂(CH₂)₃Cl |
| 1-192 | 3-F | Br | H | N | OSO₂(CH₂)₃Cl |
| 1-193 | 3-F | Cl | H | N | OSO₂(CH₂)₃Cl |
| 1-194 | 6-F | F | H | CH | 5-Cl-pyrimidin-2-oxy |
| 1-195 | 3-F | Cl | H | N | O(CH₂)₃CF₃ |
| 1-196 | H | Cl | H | N | SO(CH₂)₃CF₃ |
| 1-197 | H | Cl | H | N | SO₂(CH₂)₃CF₃ |
| 1-198 | H | Cl | H | N | S(CH₂)₃Cl |
| 1-199 | H | Cl | H | N | SO(CH₂)₃Cl |
| 1-200 | H | Cl | H | N | SO₂(CH₂)₃Cl |
| 1-201 | H | Cl | H | N | 5-F-pyrimidin-2-yl-sulfanyl |
| 1-202 | H | F | H | N | 5-F-pyrimidin-2-yl-sulfanyl |
| 1-203 | H | F | H | N | SO(CH₂)₃CF₃ |
| 1-204 | H | F | H | N | SO₂(CH₂)₃CF₃ |
| 1-205 | H | F | H | N | SO(CH₂)₃Cl |
| 1-206 | H | F | H | N | SO₂(CH₂)₃Cl |
| 1-207 | 3-Br | F | CN | N | 4-(CF₃)-pyrazol-1-yl |
| 1-208 | 3-Br | Cl | H | N | 4-(CF₃)-pyrazol-1-yl |
| 1-209 | 3-Br | F | H | N | 4-(CF₃)-pyrazol-1-yl |

In analogy to the preparation examples cited above and recited at the appropriate point, the compounds of the general formula specified hereinafter and shown in Table 2 are obtained.

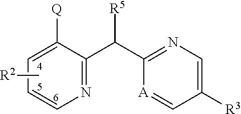

TABLE 2

| Example | | | | | |
|---|---|---|---|---|---|
| number | R² | R³ | R⁵ | A | Q |
| 2-1 | 4,6-Me₂ | F | H | N | 3-(CHF₂)-isoxazol-5-yl |
| 2-2 | 4,6-Me₂ | Cl | H | N | 3-(CHF₂)-isoxazol-5-yl |
| 2-3 | 6-Me | F | H | N | 4-(CF₃)-pyrazol-1-yl |
| 2-4 | 6-Me | Cl | H | N | 4-(CF₃)-pyrazol-1-yl |
| 2-5 | 4,6-Me₂ | F | CN | N | 3-(CHF₂)-isoxazol-5-yl |
| 2-6 | 4,6-Me₂ | Cl | CN | N | 3-(CHF₂)-isoxazol-5-yl |
| 2-7 | 6-Me | F | H | N | 5-Cl-pyrimidin-2-oxy |
| 2-8 | 6-Me | F | H | N | 5-F-pyrimidin-2-oxy |
| 2-9 | 6-Me | F | H | N | O(CH₂)₃CF₃ |
| 2-10 | 6-Me | F | H | N | OSO₂(CH₂)₃CF₃ |
| 2-11 | 6-Me | Cl | H | N | OSO₂(CH₂)₃CF₃ |
| 2-12 | 6-Me | Cl | H | N | 5-F-pyrimidin-2-oxy |
| 2-13 | 6-Me | Cl | H | N | 5-Cl-pyrimidin-2-oxy |
| 2-14 | 6-Me | Cl | H | N | O(CH₂)₃CF₃ |

The NMR data of disclosed examples are listed either in conventional form (6 values, number of hydrogen atoms, multiplet splitting) or as so-called NMR peak lists. In the NMR peak list method, the NMR data of selected examples are recorded in the form of NMR peak lists, where first the 6 value in ppm is listed for each signal peak, and then, separated by a space, the signal intensity. The 6 value-signal intensity number pairs for different signal peaks are listed with separation from one another by semicolons.

The peak list for one example therefore takes the form of:

δ₁(intensity₁);δ₂(intensity₂); . . . ;δᵢ(intensityᵢ); . . . ;δₙ(intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of H NMR peaks show the standard solvent peaks, for example peaks of DMSO in d₆-DMSO and the peak of water, which usually have a high intensity on average.

Such stereoisomers and/or impurities may be typical of the particular preparation process. Their peaks can thus help in this case to identify reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of ¹H NMR peak lists can be found in Research Disclosure Database Number 564025.

1-8: ¹H-NMR (400.6 MHz, CDCl₃):
δ = 8.5935 (16.0); 7.2666 (2.8); 7.2593 (13.1); 7.2472 (3.2); 7.2422 (1.2); 7.2274 (1.2); 7.2231 (0.9); 6.9818 (1.2); 6.9791 (1.3);
6.9631 (1.9); 6.9606 (2.2); 6.9446 (1.0); 6.9419 (1.0); 6.8306 (1.9); 6.8107 (1.8); 4.2924 (10.2); 3.9605 (2.0); 3.9460 (3.8);
3.9315 (2.1); 2.1705 (1.0); 2.1561 (0.8); 2.1511 (1.0); 2.1433 (1.4); 2.1303 (1.4); 2.1240 (1.0); 2.1161 (0.9); 2.1033 (1.3);
1.9217 (0.8); 1.9075 (1.9); 1.9014 (0.8); 1.8935 (1.4); 1.8819 (1.4); 1.8672 (1.2); 1.8531 (0.5); 1.5572 (5.5); 0.0079
(0.5); −0.0002 (19.7); −0.0085 (0.6)
1-30: ¹H-NMR (400.6 MHz, CDCl₃):
δ = 8.7038 (0.6); 8.5508 (16.0); 7.5383 (0.9); 7.5336 (1.0); 7.5197 (1.1); 7.5150 (1.1); 7.2971 (0.6); 7.2814 (1.6); 7.2781 (1.3);
7.2638 (2.6); 7.2599 (15.8); 7.2451 (2.6); 7.2262 (0.9); 6.9087 (2.3); 6.9028 (0.7); 6.8916 (0.9); 6.8886 (2.5); 6.8857 (3.0);
6.8830 (0.8); 6.8717 (0.7); 6.8657 (2.7); 6.6096 (2.8); 6.6036 (0.8); 6.5989 (2.8); 6.5928 (1.3); 6.5866 (2.3); 6.5819 (0.8);
6.5760 (2.3); 5.6286 (5.7); 2.0636 (12.7); 1.5742 (2.8); 0.0080 (0.6); −0.0002 (22.1); −0.0085 (0.6)
1-31: ¹H-NMR (400.6 MHz, CDCl₃):
δ = 8.4912 (16.0); 7.2597 (13.4); 7.2084 (0.7); 7.2026 (0.8); 7.1905 (1.3); 7.1857 (2.5); 7.1747 (2.6); 7.1692 (1.0); 7.1612 (4.2);
7.1443 (1.7); 7.1252 (0.7); 6.8735 (2.9); 6.8675 (0.9); 6.8563 (1.1); 6.8531 (3.3); 6.8504 (3.6); 6.8473 (1.0); 6.8361 (1.0);
6.8301 (3.4); 6.5935 (3.5); 6.5875 (1.0); 6.5827 (3.6); 6.5766 (1.8); 6.5705 (2.8); 6.5656 (1.0); 6.5597 (2.8); 4.1887 (10.7);
2.0819 (15.9); 1.5606 (3.2); 0.0080 (0.6); −0.0002 (21.5); −0.0085 (0.6)
1-32: ¹H-NMR (400.6 MHz, CDCl₃):
δ = 8.5384 (11.9); 7.2942 (2.4); 7.2855 (2.1); 7.2778 (6.4); 7.2658 (0.5); 7.2602 (5.9); 7.0347 (1.2); 7.0267 (1.1); 7.0185 (0.9);
7.0104 (1.0); 6.8871 (1.8); 6.8811 (0.6); 6.8700 (0.7); 6.8669 (2.0); 6.8640 (2.4); 6.8610 (0.7); 6.8499 (0.6); 6.8438 (2.2);
6.6412 (2.2); 6.6351 (0.7); 6.6304 (2.3); 6.6242 (1.1); 6.6181 (1.8); 6.6134 (0.6); 6.6074 (1.7); 5.6813 (5.0); 3.7180 (16.0);
1.5507 (1.9); −0.0002 (9.1)
1-33: ¹H-NMR (400.6 MHz, CDCl₃):
δ = 8.5451 (0.5); 8.5400 (0.5); 8.5311 (1.1); 8.5256 (1.1); 8.4703 (9.9); 7.8904 (0.6); 7.8854 (0.7); 7.7775 (1.2); 7.7721 (1.2);
7.2599 (6.7); 7.2387 (0.6); 7.2255 (0.6); 7.2165 (1.8); 7.2032 (0.8); 7.1967 (2.0); 7.1767 (1.6); 7.0271 (0.9); 7.0057 (1.4);
6.9834 (0.7); 6.9717 (1.0); 6.9681 (1.3); 6.9523 (1.0); 6.9487 (1.0); 6.9417 (1.3); 6.9383 (1.0); 6.9212 (1.1); 6.9177 (0.9);
6.8561 (1.7); 6.8501 (0.6); 6.8389 (0.7); 6.8356 (2.0); 6.8330 (2.3); 6.8298 (0.6); 6.8187 (0.6); 6.8127 (2.1); 6.6308 (2.2);
6.6247 (0.7); 6.6199 (2.3); 6.6138 (1.1); 6.6077 (1.7); 6.6028 (0.6); 6.5969 (1.7); 5.8979 (1.4); 5.4981 (1.4); 4.2343 (6.6);
3.7303 (16.0); 1.2555 (0.5); −0.0002 (10.4)
1-34: ¹H-NMR (400.6 MHz, CDCl₃):
δ = 8.5742 (16.0); 7.4893 (0.8); 7.4860 (1.4); 7.4826 (0.8); 7.4697 (1.0); 7.4664 (1.7); 7.4631 (1.0); 7.3097 (0.8); 7.2970 (0.8);
7.2890 (1.4); 7.2765 (1.4); 7.2694 (1.2); 7.2602 (8.8); 7.2567 (1.2); 7.2316 (1.2); 7.2275 (1.2); 7.2107 (0.8); 7.2064 (1.8);
7.2023 (1.2); 7.1855 (0.8); 7.1815 (0.7); 6.9401 (2.4); 6.9340 (0.8); 6.9229 (1.1); 6.9200 (2.7); 6.9170 (3.4); 6.9142 (0.9);
6.9031 (0.9); 6.8970 (3.1); 6.7468 (1.9); 6.7456 (2.0); 6.7409 (0.6); 6.7363 (1.9); 6.7349 (1.9); 6.7301 (1.0); 6.7285 (1.0);
6.7238 (1.6); 6.7194 (0.6); 6.7177 (0.6); 6.7132 (1.6); 5.7416 (5.8); 2.0451 (0.5); 1.5480 (4.3); 1.2645 (0.6); 1.2593 (0.7);
0.8818 (1.2); −0.0002 (13.2)
1-35: ¹H-NMR (400.6 MHz, CDCl₃):
δ = 8.4948 (16.0); 7.2591 (3.2); 7.1849 (1.7); 7.1729 (1.5); 7.1656 (2.1); 7.1550 (3.6); 7.1505 (3.2); 7.1362 (0.7); 7.1179 (1.3);
7.1126 (1.0); 7.0986 (0.7); 7.0928 (1.7); 7.0859 (1.1); 7.0746 (0.7); 7.0680 (0.7); 6.9020 (2.8); 6.8959 (1.0); 6.8849 (1.2);
6.8818 (3.2); 6.8788 (4.0); 6.8758 (1.1); 6.8648 (1.1); 6.8586 (3.7); 6.7225 (2.4); 6.7177 (0.8); 6.7160 (0.8); 6.7129 (2.3);
6.7117 (2.3); 6.7068 (1.2); 6.7053 (1.1); 6.7006 (1.8); 6.6960 (0.7); 6.6944 (0.7); 6.6898 (1.9); 4.2929 (11.6); −0.0002 (5.0)
1-36: ¹H-NMR (400.6 MHz, CDCl₃):
δ = 8.5542 (16.0); 7.7121 (1.4); 7.7083 (1.9); 7.6917 (2.0); 7.6896 (3.4); 7.6862 (1.9); 7.6698 (2.6); 7.6660 (1.8); 7.2660 (2.2);
7.2601 (8.7); 7.2462 (3.5); 7.2264 (1.8); 6.9304 (2.5); 6.9244 (0.8); 6.9132 (1.1); 6.9104 (2.9); 6.9073 (3.2); 6.9046 (0.9);
6.8934 (0.8); 6.8873 (2.9); 6.6307 (3.1); 6.6246 (0.9); 6.6201 (3.1); 6.6138 (1.5); 6.6076 (2.4); 6.6030 (0.9); 6.5971 (2.4);
5.6483 (5.7); 1.5471 (3.9); −0.0002 (13.1)
1-37: ¹H-NMR (400.6 MHz, CDCl₃):
δ = 8.4932 (16.0); 7.5783 (2.2); 7.5747 (2.3); 7.5583 (2.5); 7.5547 (2.5); 7.3411 (2.0); 7.3388 (2.0); 7.3373 (2.0); 7.3220 (2.5);
7.3203 (2.4); 7.3183 (2.4); 7.2605 (8.0); 7.1500 (2.4); 7.1305 (3.8); 7.1109 (1.9); 6.8987 (3.0); 6.8928 (1.2); 6.8884 (0.7);
6.8784 (4.6); 6.8758 (4.6); 6.8658 (0.8); 6.8614 (1.3); 6.8555 (3.5); 6.6290 (3.8); 6.6229 (1.9); 6.6183 (3.9); 6.6122 (2.3);
6.6060 (3.2); 6.6013 (1.6); 6.5954 (3.0); 4.2303 (12.8); 1.5673 (10.3); 1.5618 (3.0); 0.0078 (0.6); −0.0002 (12.5)
1-38: ¹H-NMR (400.6 MHz, CDCl₃):
δ = 8.5194 (16.0); 7.6424 (1.3); 7.6383 (2.1); 7.6292 (2.4); 7.6248 (2.2); 7.6191 (2.5); 7.6097 (2.9); 7.6055 (1.5); 7.3387 (2.5);
7.3193 (4.2); 7.3000 (1.9); 7.2606 (19.8); 6.9387 (3.0); 6.9327 (1.0); 6.9215 (1.3); 6.9186 (3.4); 6.9156 (4.1); 6.9128 (1.2);
6.9017 (1.1); 6.8957 (3.8); 6.7203 (3.9); 6.7143 (1.2); 6.7098 (4.0); 6.7035 (2.0); 6.6973 (3.1); 6.6927 (1.1); 6.6867 (3.0);
4.2657 (11.5); 1.5936 (0.9); 1.2555 (0.6); 0.0080 (0.8); −0.0002 (31.4); −0.0085 (0.9)
1-39: ¹H-NMR (400.6 MHz, CDCl₃):
δ = 8.5849 (9.8); 8.0022 (0.7); 7.9984 (0.7); 7.9823 (0.8); 7.9787 (0.8); 7.7902 (0.8); 7.7733 (0.8); 7.7705 (0.8); 7.5115 (0.6);
7.4919 (1.0); 7.4721 (0.5); 7.2601 (30.9); 6.9373 (1.5); 6.9174 (1.6); 6.9142 (1.9); 6.8944 (1.8); 6.6568 (1.7); 6.6464 (1.7);
6.6401 (0.8); 6.6338 (1.4); 6.6234 (1.4); 5.5496 (3.6); 3.5556 (0.6); 1.5372 (16.0); 1.2646 (0.8); 0.8820 (1.6); 0.8643 (0.6);
0.0080 (1.2); −0.0002 (47.4); −0.0085 (1.4)
1-40: ¹H-NMR (400.6 MHz, CDCl₃):
δ = 8.5846 (2.7); 8.5170 (16.0); 7.6642 (1.7); 7.6447 (1.9); 7.5634 (1.6); 7.5454 (2.0); 7.3682 (1.4); 7.3489 (2.3); 7.3296 (1.0);
7.2602 (9.7); 6.9172 (0.6); 6.9141 (0.6); 6.8945 (0.7); 6.8851 (2.9); 6.8792 (1.0); 6.8649 (3.8); 6.8622 (4.2); 6.8522 (0.5);
6.8479 (1.1); 6.8419 (3.4); 6.6572 (0.6); 6.6465 (0.7); 6.6355 (3.8); 6.6294 (1.6); 6.6248 (3.8); 6.6187 (1.9); 6.6127 (2.8);
6.6079 (1.2); 6.6021 (2.8); 5.5507 (1.1); 4.1416 (12.1); 1.5578 (5.1); −0.0002 (14.4); −0.0084 (0.6)
1-51: ¹H-NMR (400.6 MHz, CDCl₃):
δ = 8.6534 (0.6); 8.4811 (11.9); 8.4030 (16.0); 7.3662 (0.6); 7.3503 (0.6); 7.3455 (1.3); 7.3297 (1.3); 7.3248 (0.8); 7.3090 (0.8);
7.2604 (18.0); 7.0931 (0.8); 7.0904 (0.8); 7.0703 (1.3); 7.0497 (0.7); 7.0471 (0.7); 6.9855 (0.9); 6.9828 (1.6); 6.9650 (0.8);
6.9623 (1.4); 4.3211 (4.7); 4.3191 (4.8); 0.0080 (0.8); −0.0002 (26.2); −0.0085 (0.8)
1-52: ¹H-NMR (400.0 MHz, CDCl₃):
δ = 8.4874 (15.2); 8.3362 (16.0); 7.3623 (1.1); 7.3423 (2.8); 7.3217 (2.8); 7.3054 (1.3); 7.2602 (22.3); 7.0809 (2.0); 7.0607 (3.8);
7.0388 (1.8); 6.9812 (3.8); 6.9607 (3.4); 4.3277 (14.9); 2.0456 (0.6); 1.5511 (7.9); 1.2578 (0.6); −0.0002 (32.1)
1-53: ¹H-NMR (400.6 MHz, CDCl₃):
δ = 8.6539 (1.6); 8.4959 (9.4); 8.4161 (12.0); 7.5732 (1.3); 7.5703 (1.2); 7.5532 (1.5); 7.5503 (1.4); 7.2692 (1.2); 7.2603 (26.8);
7.2490 (2.6); 7.2288 (1.5); 7.1483 (1.6); 7.1455 (1.5); 7.1279 (1.1); 7.1251 (1.0); 4.5010 (6.9); 1.5457 (16.0); 0.0079
(0.9); −0.0002 (29.6); −0.0085 (1.1)
1-54: ¹H-NMR (400.6 MHz, CDCl₃):
δ = 8.5010 (14.4); 8.3471 (16.0); 7.5655 (2.2); 7.5629 (2.1); 7.5456 (2.5); 7.5429 (2.4); 7.2670 (2.1); 7.2603 (25.0); 7.2471 (4.1);
7.2269 (2.3); 7.1496 (2.7); 7.1472 (2.6); 7.1293 (1.9); 4.5088 (12.2); 1.5484 (12.3); 0.0078 (1.1); −0.0002 (27.2); −0.0083 (1.4)
1-55: ¹H-NMR (400.6 MHz, CDCl₃):
δ = 8.6053 (1.4); 8.5087 (13.2); 7.7817 (3.6); 7.7762 (3.8); 7.5513 (1.8); 7.5485 (1.8); 7.5313 (2.2); 7.5285 (2.0); 7.4680 (2.3);
7.4626 (2.0); 7.4456 (2.1); 7.4401 (2.0); 7.2600 (32.2); 7.2446 (1.9); 7.2243 (3.5); 7.2171 (0.6); 7.2072 (1.1); 7.2041 (2.0);
7.1113 (2.2); 7.1085 (2.1); 7.0909 (1.7); 7.0882 (1.5); 6.0097 (0.5); 4.5072 (10.1); 1.5440 (16.0); 1.3053 (0.6); 1.2881 (0.6);
0.0079 (1.2); −0.0002 (35.1); −0.0085 (1.4)
1-56: ¹H-NMR (400.6 MHz, CDCl₃):
δ = 8.6665 (0.8); 8.6526 (2.5); 8.4919 (13.3); 8.4166 (16.0); 7.3932 (1.2); 7.3901 (1.4); 7.3730 (2.4); 7.3699 (2.4); 7.3329 (2.2);
7.3127 (3.4); 7.2926 (1.5); 7.2607 (10.4); 7.1088 (1.9); 7.1057 (2.0); 7.0887 (1.7); 7.0855 (1.7); 4.5396 (0.6); 4.4688 (10.2);
1.5691 (3.7); −0.0002 (11.4)
1-57: ¹H-NMR (400.6 MHz, CDCl₃):
δ = 8.4974 (14.3); 8.3480 (16.0); 7.3860 (1.3); 7.3829 (1.5); 7.3658 (2.8); 7.3627 (2.8); 7.3309 (2.4); 7.3108 (3.7); 7.2906 (1.6);
7.2610 (8.9); 7.1102 (2.2); 7.1071 (2.3); 7.0901 (1.9); 7.0871 (1.9); 4.4769 (12.0); 1.5731 (3.1); −0.0002 (9.8); −0.0083 (0.6)
1-58: ¹H-NMR (400.6 MHz, CDCl₃):
δ = 8.6051 (2.7); 8.5054 (14.6); 8.2917 (0.7); 8.2864 (0.7); 7.7789 (4.1); 7.7734 (4.4); 7.4725 (2.5); 7.4670 (2.4); 7.4500 (2.4);
7.4445 (2.4); 7.3723 (1.4); 7.3692 (1.5); 7.3522 (2.7); 7.3490 (2.5); 7.3085 (2.3); 7.2883 (3.8); 7.2820 (0.6); 7.2681 (1.8);
7.2602 (26.2); 7.1743 (0.8); 7.1719 (0.6); 7.1534 (0.5); 7.0712 (2.1); 7.0680 (2.1); 7.0510 (1.8); 7.0479 (1.8); 6.0263 (0.8);
5.2995 (1.8); 4.4743 (10.8); 1.5494 (16.0); 0.0079 (0.8); −0.0002 (28.6); −0.0085 (1.1)
1-59: ¹H-NMR (400.6 MHz, CDCl₃):
δ = 8.5012 (2.7); 8.4897 (13.4); 8.3474 (3.2); 8.3217 (16.0); 7.6656 (1.7); 7.6624 (1.8); 7.6466 (2.2); 7.6434 (2.1); 7.4907 (1.5);
7.4703 (2.8); 7.4510 (2.1); 7.4048 (2.4); 7.4015 (2.3); 7.3842 (1.5); 7.3810 (1.3); 7.2610 (22.6); 7.2474 (0.9); 7.1502 (0.5);
7.1474 (0.5); 5.3000 (5.4); 4.5370 (10.3); 4.5091 (2.2); 1.5564 (7.2); 0.0079 (0.8); −0.0002 (24.7); −0.0068 (0.9); −0.0084 (1.2)
1-60: ¹H-NMR (400.6 MHz, CDCl₃):
δ = 8.5813 (16.0); 7.2598 (17.3); 7.2388 (0.8); 7.2220 (0.9); 7.2179 (1.8); 7.2012 (1.8); 7.1971 (1.1); 7.1804 (1.0); 6.7924 (1.0);
6.7902 (1.1); 6.7692 (1.9); 6.7491 (1.0); 6.7468 (1.0); 6.6467 (1.9); 6.6259 (1.8); 4.3464 (6.4); 4.3441 (6.6); 3.9739 (1.9);
3.9593 (3.6); 3.9447 (2.0); 2.1342 (0.9); 2.1191 (0.8); 2.1149 (0.9); 2.1072 (1.3); 2.0941 (1.3); 2.0882 (0.9); 2.0800 (0.9);
2.0672 (1.2); 1.9222 (0.8); 1.9080 (1.9); 1.9019 (0.7); 1.8941 (1.4); 1.8886 (1.0); 1.8831 (1.3); 1.8678 (1.1); 0.0080
(0.7); −0.0002 (26.1); −0.0085 (0.7)

NMR data of the end products (conventional form):

The ¹H NMR spectroscopic data given for the chemical examples described in the following sections (400 MHz for ¹H NMR, solvent CDCl₃ or d₆-DMSO, internal standard: tetramethylsilane δ=0.00 ppm) were obtained on a Bruker instrument, and the signals listed have the meanings given below: b=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, what is reported is either the significant signals for each of the two diastereomers or the characteristic signal of the main diastereomer.

1-1: ¹H-NMR (400.0 MHz, CDCl₃):
δ = 8.60 (s, 2H); 7.29 (dd, 1H); 7.22-7.15 (m, 3H); 4.30 (s, 2H); 2.78 (m, 2H); 2.10 (m, 2H); 1.79 (m, 2H)
1-3: ¹H-NMR (400.0 MHz, CDCl₃):
δ = 8.51 (s, 2H); 7.29 (dd, 1H); 7.22-7.15 (m, 3H); 4.32 (s, 2H); 2.78 (m, 2H); 2.10 (m, 2H); 1.79 (m, 2H)
1-10: ¹H-NMR (400.0 MHz, CDCl₃):
δ = 8.60 (s, 2H); 7.42-7.27 (m, 4H); 4.37 (s, 2H); 3.46 (t, 2H); 2.40-2.26 (m, 4H)
1-14: ¹H-NMR (400.0 MHz, CDCl₃):
δ = 8.62 (s, 2H); 7.27-7.21 (m, 1H); 7.14-7.12 (m, 2H); 4.46 (s, 2H); 3.56-3.48 (m, 2H); 2.41-2.17 (m, 4H)
1-15: ¹H-NMR (400.0 MHz, CDCl₃):
δ = 8.53 (s, 2H); 7.25-7.21 (m, 1H); 7.14-7.09 (m, 2H); 4.48 (s, 2H); 3.56-3.52 (m, 2H); 2.41-2.27 (m, 4H)
1-16: ¹H-NMR (400.0 MHz, CDCl₃):
δ = 8.62 (s, 2H); 7.28-7.23 (m, 1H); 7.16-7.11 (m, 2H); 4.45 (s, 2H); 3.71-3.67 (m, 2H); 2.93-2.84 (m, 2H)
1-17: ¹H-NMR (400.0 MHz, CDCl₃):
δ = 8.53 (s, 2H); 7.26-7.23 (m, 1H); 7.16-7.11 (m, 2H); 4.47 (s, 2H); 3.71-3.67 (m, 2H); 2.90-2.84 (m, 2H)
1-18: ¹H-NMR (400.0 MHz, CDCl₃):
δ = 8.62 (s, 2H); 7.39-7.37 (m, 1H); 7.24-7.18 (m, 2H); 4.51 (s, 2H); 3.67-3.63 (t, 2H); 2.45-2.30 (m, 4H)
1-19: ¹H-NMR (400.0 MHz, CDCl₃):
δ = 8.53 (s, 2H); 7.39-7.37 (m, 1H); 7.24-7.18 (m, 2H); 4.52 (s, 2H); 3.67-3.63 (t, 2H); 2.43-2.29 (m, 4H)
1-20: ¹H-NMR (400.0 MHz, CDCl₃):
δ = 8.62 (s, 2H); 7.40-7.38 (m, 1H); 7.27-7.20 (m, 2H); 4.49 (s, 2H); 3.82-3.78 (m, 2H); 2.96-2.84 (m, 2H)
1-21: ¹H-NMR (400.0 MHz, CDCl₃):
δ = 8.53 (s, 2H); 7.40-7.38 (m, 1H); 7.27-7.20 (m, 2H); 4.50 (s, 2H); 3.82-3.78 (m, 2H); 2.96-2.84 (m, 2H)
1-23: ¹H-NMR (400.0 MHz, CDCl₃):
δ = 8.53 (s, 2H); 7.31-7.22 (m, 3H); 7.11 (m, 1H); 6.99 (m, 2H); 6.90 (m, 2H); 4.25 (s, 2H); 4.05 (s, 2H)
1-26: ¹H-NMR (400.0 MHz, CDCl₃):
δ = 8.57 (s, 2H); 7.34-7.18 (m, 5H); 7.13 (m, 1H); 7.07 (m, 1H); 4.25 (s, 2H)
1-27: ¹H-NMR (400.0 MHz, CDCl₃):
δ = 8.48 (s, 2H); 7.34-7.18 (m, 5H); 7.13 (m, 1H); 7.06 (m, 1H); 4.27 (s, 2H)
1-29: ¹H-NMR (400.0 MHz, CDCl₃):
δ = 8.55 (s, 2H); 7.33 (dd, 1H); 7.23 (ddd, 1H); 7.11 (dd, 1H); 6.94 (m, 2H); 6.85-6.79 (m, 3H); 4.35 (s, 2H)
1-41: ¹H-NMR (400.0 MHz, CDCl₃):
δ = 8.61 (s, 2H); 7.91 (s, 2H); 7.82 (dd, 1H); 7.59-7.51 (m, 2H); 7.38 (dd, 1H); 6.04 (s, 1H)
1-42: ¹H-NMR (400.0 MHz, CDCl₃):
δ = 8.54 (s, 2H); 8.00 (m, 1H); 7.84 (s, 1H); 7.46-7.36 (m, 4H); 4.29 (s, 2H)
1-45: ¹H-NMR (400.0 MHz, CDCl₃):
δ = 8.54 (s, 2H); 7.98 (m, 1H); 7.95 (s, 1H); 7.75 (dd, 1H); 7.73 (dd, 1H); 7.59 (dd, 1H); 4.20 (s, 2H)
1-50: ¹H-NMR (400.0 MHz, CDCl₃):
δ = 8.48 (s, 2H); 8.40 (s, 2H); 7.43 (dd, 1H); 7.35 (ddd, 1H); 7.28 (dd, 1H); 7.13 (d, 1H); 4.26 (s, 2H)
1-64: ¹H-NMR (400.0 MHz, CDCl₃):
δ = 8.45 (s, 2H); 8.39 (s, 2H); 7.26 (dd, 1H); 7.16 (d, 1H); 6.99 (dd, 1H); 4.32 (s, 2H); 2.40 (s, 3H)
1-65: ¹H-NMR (400.0 MHz, CDCl₃):
δ = 8.46 (s, 2H); 8.32 (s, 2H); 7.29-7.24 (m, 1H); 7.14 (dd, 1H); 6.99 (d, 1H); 4.33 (s, 2H); 2.39 (s, 3H)
1-66: ¹H-NMR (400.0 MHz, CDCl₃):
δ = 8.47 (s, 2H); 7.72 (d, 1H); 7.44 (dd, 1H); 7.24 (dd, 1H); 7.14 (d, 1H); 6.95 (d, 1H); 4.30 (s, 2H); 2.41 (s, 3H)
1-67: ¹H-NMR (400.0 MHz, CDCl₃):
δ = 8.47 (s, 2H); 7.33 (dd, 1H); 7.22 (ddd, 1H); 7.11 (ddd, 1H); 6.94 (m, 2H); 6.85-6.79 (m, 3H); 4.37 (s, 2H)
1-68: ¹H-NMR (400.0 MHz, CDCl₃):
δ = 8.68 (s, 2H); 7.60 (dd, 1H); 7.34-7.28 (m, 2H); 7.22 (dd, 1H); 5.61 (s, 1H); 2.87 (m, 2H); 2.17 (m, 2H); 1.87 (m, 2H)
1-69: ¹H-NMR (400.0 MHz, CDCl₃):
δ = 8.60 (s, 2H); 7.65 (m, 1H); 7.37-7.31 (m, 2H); 7.15 (m, 1H); 7.01-6.92 (m, 4H); 5.56 (s, 1H); 4.25 (d, 1H); 4.07 (d, 1H)
1-70: ¹H-NMR (400.0 MHz, CDCl₃):
δ = 8.41 (s, 2H); 8.40 (s, 2H); 7.43 (dd, 1H); 7.35 (ddd, 1H); 7.28 (ddd, 1H); 7.13 (dd, 1H); 4.28 (s, 2H)
1-71: ¹H-NMR (400.0 MHz, CDCl₃):
δ = 8.60 (s, 2H); 8.14 (dd, 1H); 8.00 (s, 1H); 7.94 (s, 1H); 7.87 (dd, 1H); 7.74 (dd, 1H); 5.73 (s, 1H)
1-72: ¹H-NMR (400.0 MHz, CDCl₃):
δ = 8.59 (s, 2H); 7.61 (dd, 1H); 7.36-7.28 (m, 2H); 7.22 (dd, 1H); 5.64 (s, 1H); 2.86 (m, 2H); 2.17 (m, 2H); 1.86 (m, 2H)
1-73: ¹H-NMR (400.0 MHz, CDCl₃):
δ = 8.54 (dd, 1H); 7.67 (dd, 1H); 7.48 (dd, 1H); 7.40-7.21 (m, 4H); 5.47 (s, 1H); 2.73 (m, 2H); 2.11 (m, 2H); 1.78 (m, 2H)
1-74: ¹H-NMR (400.0 MHz, CDCl₃):
δ = 8.46 (s, 1H); 7.50 (d, 1H); 7.31 (d, 1H); 7.20 (dd, 1H); 7.08 (m, 2H); 6.97 (m, 2H); 6.68-6.83 (m, 3H); 4.16 (s, 2H)
1-75: ¹H-NMR (400.0 MHz, CDCl₃):
δ = 8.55 (d, 2H); 8.31 (d, 2H); 7.44 (dd, 1H); 7.34 (ddd, 1H); 7.27 (ddd, 1H); 7.13 (dd, 1H); 7.04 (t, 1H); 4.29 (s, 2H)
1-76: ¹H-NMR (400.0 MHz, CDCl₃):
δ = 8.53 (d, 2H); 8.38 (s, 2H); 7.45 (dd, 1H); 7.34 (ddd, 1H); 7.28 (ddd, 1H); 7.13 (dd, 1H); 7.05 (t, 1H); 4.28 (s, 2H)
1-77: ¹H-NMR (400.0 MHz, CDCl₃):
δ = 8.55 (d, 2H); 7.73 (d, 1H); 7.46-7.43 (m, 2H); 7.32 (ddd, 1H); 7.26 (ddd, 1H); 7.10 (dd, 1H); 7.05 (t, 1H); 4.27 (s, 2H)
1-78: ¹H-NMR (400.0 MHz, CDCl₃):
δ = 8.68 (s, 2H); 7.62 (m, 1H); 7.44-7.38 (m, 2H); 7.31-7.22 (m, 3H); 7.16 (m, 1H); 5.53 (s, 1H)
1-79: ¹H-NMR (400.0 MHz, CDCl₃):
δ = 8.58 (s, 2H); 7.63 (m, 1H); 7.44-7.38 (m, 2H); 7.32-7.22 (m, 3H); 7.15 (m, 1H); 5.56 (s, 1H)
1-80: ¹H-NMR (400.0 MHz, CDCl₃):
δ = 8.50 (dd, 1H); 7.64 (dd, 1H); 7.52 (dd, 1H); 7.46-7.38 (m, 2H); 7.28-7.14 (m, 4H); 7.06 (m, 1H); 5.36 (s, 1H)
1-81: ¹H-NMR (400.0 MHz, CDCl₃):
δ = 8.52 (s, 2H); 7.66 (m, 1H); 7.37-7.29 (m, 2H); 7.15 (m, 1H); 7.03-6.93 (m, 4H); 5.59 (s, 1H); 4.25 (d, 1H); 4.07 (d, 1H)
1-82: ¹H-NMR (400.0 MHz, CDCl₃):
δ = 8.51 (dd, 1H); 7.58-7.52 (m, 2H); 7.36-7.30 (m, 2H); 7.18 (m, 1H); 7.05-6.92 (m, 5H); 5.41 (s, 1H); 4.06 (d, 1H); 4.00 (d, 1H)
1-83: ¹H-NMR (400.0 MHz, CDCl₃):
δ = 8.75 (s, 2H); 7.58 (d, 1H); 7.32 (dd, 1H); 7.16 (dd, 1H); 7.03 (m, 2H); 6.98 (m, 2H); 6.80 (m, 1H); 5.86 (s, 1H)
1-84: ¹H-NMR (400.0 MHz, CDCl₃):
δ = 8.54 (s, 1H); 7.63 (d, 1H); 7.57 (d, 1H); 7.35 (d, 1H); 7.28 (dd, 1H); 7.15 (dd, 1H); 7.02 (m, 2H); 6.88 (m, 2H); 6.76 (d, 1H); 5.68
(s, 1H)
1-85: ¹H-NMR (400.0 MHz, CDCl₃):
δ = 8.75 (d, 1H); 7.90 (d, 1H); 7.68 (dd, 1H); 7.30 (m, 1H); 7.16 (ddd, 1H); 7.02 (m, 2H); 6.88 (m, 2H); 6.75 (dd, 1H); 6.13 (s, 1H)
1-86: ¹H-NMR (400.0 MHz, CDCl₃):
δ = 8.63 (s, 2H); 7.71 (m, 1H); 7.42-7.38 (m, 2H); 7.33 (m, 1H); 7.18 (m, 2H); 6.97 (m, 2H); 6.22 (s, 1H)
1-87: ¹H-NMR (400.0 MHz, CDCl₃):
δ = 8.64 (s, 2H); 7.33 (dd, 1H); 7.23 (ddd, 1H); 7.13 (ddd, 1H); 6.94 (m, 2H); 6.85-6.79 (m, 3H); 4.33 (s, 2H)
1-88: ¹H-NMR (400.0 MHz, CDCl₃):
δ = 8.55 (s, 2H); 7.71 (m, 1H); 7.41-7.30 (m, 3H); 7.19 (m, 2H); 6.97 (m, 2H); 6.24 (s, 1H)
1-89: ¹H-NMR (400.0 MHz, CDCl₃):
δ = 8.41 (s, 2H); 8.34 (s, 2H); 7.42 (dd, 1H); 7.35 (ddd, 1H); 7.27 (ddd, 1H); 7.13 (dd, 1H); 4.29 (s, 2H)
1-90: ¹H-NMR (400.0 MHz, CDCl₃):
δ = 8.41 (s, 2H); 7.75 (d, 1H); 7.46 (dd, 1H); 7.43 (dd, 1H); 7.33 (ddd, 1H); 7.26 (ddd, 1H); 7.10 (dd, 1H); 4.27 (s, 2H)
1-91: ¹H-NMR (400.0 MHz, CDCl₃):
δ = 8.41 (s, 2H); 8.38 (m, 1H); 7.45 (dd, 1H); 7.37-7.32 (m, 2H); 7.26 (ddd, 1H); 7.12 (dd, 1H); 7.03 (d, 1H); 4.08 (s, 2H)
1-92: ¹H-NMR (400.0 MHz, CDCl₃):
δ = 8.41 (s, 2H); 8.29 (d, 1H); 7.37-7.31 (m, 2H); 7.28-7.19 (m, 3H); 7.12 (dd, 1H); 7.07 (dd, 1H); 4.09 (s, 2H)
1-93: ¹H-NMR (400.0 MHz, CDCl₃):
δ = 8.34 (s, 2H); 8.28 (d, 1H); 7.37-7.31 (m, 2H); 7.27-7.19 (m, 3H); 7.12 (dd, 1H); 7.07 (dd, 1H); 4.10 (s, 2H)
1-94: ¹H-NMR (400.0 MHz, CDCl₃):
δ = 8.49 (d, 1H); 7.53 (dd, 1H); 7.23-7.17 (m, 4H); 6.93 (d, 1H); 4.16 (s, 2H); 2.66 (m, 2H); 2.05 (m, 2H); 1.72 (m, 2H)
1-95: ¹H-NMR (400.0 MHz, CDCl₃):
δ = 8.45 (s, 2H); 7.29-7.21 (m, 3H); 7.11 (m, 1H); 7.00 (m, 2H); 6.90 (m, 2H); 4.27 (s, 2H); 4.06 (s, 2H)
1-96: ¹H-NMR (400.0 MHz, CDCl₃):
δ = 8.46 (dd, 1H); 7.44 (dd, 1H); 7.25-7.12 (m, 4H); 6.98 (m, 2H); 6.90 (m, 2H); 6.78 (dd, 1H); 4.10 (s, 2H); 3.93 (s, 2H)
1-97: ¹H-NMR (400.0 MHz, CDCl₃):
δ = 8.44 (dd, 1H); 7.48 (dd, 1H); 7.36-7.21 (m, 4H); 7.17-7.05 (m, 2H); 6.95 (m, 1H); 6.80 (dd, 1H); 4.08 (s, 2H)
1-98: ¹H-NMR (400.0 MHz, CDCl₃):
δ = 8.62 (d, 2H); 7.35 (ddd, 1H); 7.22 (ddd, 1H); 7.11 (ddd, 1H); 7.06 (t, 1H); 6.92 (m, 2H); 6.85-6.79 (m, 3H); 4.38 (s, 2H)
1-99: ¹H-NMR (400.0 MHz, CDCl₃):
δ = 8.56 (s, 2H); 7.35-7.20 (m, 4H); 7.15 (m, 2H); 6.93 (m, 2H); 4.50 (s, 2H)
1-100: ¹H-NMR (400.0 MHz, CDCl₃):
δ = 8.39 (m, 1H); 8.34 (s, 2H); 7.46 (dd, 1H); 7.37-7.32 (m, 2H); 7.25 (ddd, 1H); 7.12 (dd, 1H); 7.05 (d, 1H); 4.10 (s, 2H)
1-101: ¹H-NMR (400.0 MHz, CDCl₃):
δ = 8.39 (m, 1H); 7.78 (d, 1H); 7.48 (dd, 1H); 7.47 (d, 1H); 7.36-7.30 (m, 2H); 7.23 (ddd, 1H); 7.10 (dd, 1H); 7.03 (d, 1H); 4.10 (s, 2H)
1-102: ¹H-NMR (400.0 MHz, CDCl₃):
δ = 8.53 (dd, 1H); 7.67 (dd, 1H); 7.60 (dd, 1H); 7.41-7.29 (m, 3H); 7.22-7.14 (m, 3H); 6.99 (m, 2H); 6.00 (s, 1H)
1-103: ¹H-NMR (400.0 MHz, CDCl₃):
δ = 8.30 (d, 1H); 7.78 (d, 1H); 7.46 (dd, 1H); 7.37-7.32 (m, 2H); 7.25-7.20 (m, 2H); 7.09 (dd, 1H); 7.05 (dd, 1H); 4.10 (s, 2H)
1-104: ¹H-NMR (400.0 MHz, CDCl₃):
δ = 8.48 (s, 2H); 7.34-7.21 (m, 4H); 7.18 (m, 2H); 6.94 (m, 2H); 4.52 (s, 2H)
1-105: ¹H-NMR (400.0 MHz, CDCl₃):
δ = 8.48 (d, 1H); 7.50 (dd, 1H); 7.30-7.15 (m, 6H); 7.00-6.94 (m, 3H); 4.32 (s, 2H)
1-106: ¹H-NMR (400.0 MHz, CDCl₃):
δ = 8.28 (d, 1H); 7.35 (dd, 1H); 7.30-7.18 (m, 4H); 7.16 (m, 2H); 6.94 (m, 2H); 4.32 (s, 2H)
1-107: ¹H-NMR (400.0 MHz, CDCl₃):
δ = 8.69 (s, 2H); 7.64 (m, 1H); 7.56 (m, 1H); 7.40-7.36 (m, 2H); 6.30 (s, 1H); 3.15 (m, 1H); 3.05 (m, 1H); 2.38 (m, 2H)
1-108: ¹H-NMR (400.0 MHz, CDCl₃):
δ = 8.60 (s, 2H); 7.65 (m, 1H); 7.56 (m, 1H); 7.40-7.36 (m, 2H); 6.32 (s, 1H); 3.14 (m, 1H); 3.04 (m, 1H); 2.38 (m, 2H)
1-109: ¹H-NMR (400.0 MHz, CDCl₃):
δ = 8.54 (s, 1H); 7.68 (dd, 1H); 7.57 (m, 1H); 7.51 (m, 1H); 7.40-7.34 (m, 3H); 6.05 (s, 1H); 3.09 (m, 1H); 3.02 (m, 1H); 2.36 (m, 2H)
1-110: ¹H-NMR (400.0 MHz, CDCl₃):
δ = 8.31 (d, 1H); 7.43-7.41 (m, 2H); 7.27-7.17 (m, 3H); 4.36 (s, 2H); 3.02 (m, 2H); 2.33 (m, 2H)
1-111: ¹H-NMR (400.0 MHz, CDCl₃):
δ = 8.52 (s, 2H); 7.44 (m, 1H); 7.33-7.24 (m, 3H); 4.54 (s, 2H); 3.02 (m, 2H); 2.33 (m, 2H)
1-112: ¹H-NMR (400.0 MHz, CDCl₃):
δ = 8.61 (s, 2H); 7.44 (m, 1H); 7.33-7.25 (m, 3H); 4.52 (s, 2H); 3.03 (m, 2H); 2.33 (m, 2H)
1-113: ¹H-NMR (400.0 MHz, CDCl₃):
δ = 8.43 (dd, 1H); 7.65 (m, 1H); 7.53-7.34 (m, 4H); 6.26 (d, 1H); 3.04 (m, 2H); 2.34 (m, 2H)
1-114: ¹H-NMR (400.0 MHz, CDCl₃):
δ = 8.69 (s, 2H); 7.66 (dd, 1H); 7.52 (dd, 1H); 7.37-7.32 (m, 2H); 6.30 (s, 1H); 2.98 (m, 2H); 2.24 (m, 2H); 1.87 (m, 2H)
1-115: ¹H-NMR (400.0 MHz, CDCl₃):
δ = 8.60 (s, 2H); 7.62 (m, 1H); 7.52 (m, 1H); 7.37-7.32 (m, 2H); 6.32 (d, 1H); 2.98 (m, 2H); 2.24 (m, 2H); 1.87 (m, 2H)
1-116: ¹H-NMR (400.0 MHz, CDCl₃):
δ = 8.43 (dd, 1H); 7.63 (m, 1H); 7.51-7.32 (m, 4H); 6.28 (d, 1H); 2.95 (m, 2H); 2.19 (m, 2H); 1.86 (m, 2H)
1-117: ¹H-NMR (400.0 MHz, CDCl₃):
δ = 8.55 (d, 1H); 7.67 (dd, 1H); 7.56 (m, 1H); 7.48 (m, 1H); 7.38-7.29 (m, 3H); 6.05 (s, 1H); 2.94 (m, 2H); 2.19 (m, 2H); 1.85 (m, 2H)
1-118: ¹H-NMR (400.0 MHz, CDCl₃):
δ = 8.70 (s, 2H); 7.61 (m, 1H); 7.33-7.30 (m, 2H); 7.23 (m, 1H); 5.60 (s, 1H); 3.07 (m, 2H); 2.49 (m, 1H); 2.38 (m, 1H)
1-119: ¹H-NMR (400.0 MHz, CDCl₃):
δ = 8.61 (s, 2H); 7.28 (m, 1H); 7.23-7.17 (m, 3H); 4.31 (s, 2H); 2.99 (m, 2H); 2.36 (m, 2H)
1-120: ¹H-NMR (400.0 MHz, CDCl₃):
δ = 8.61 (s, 2H); 7.62 (m, 1H); 7.35-7.29 (m, 2H); 7.23 (m, 1H); 5.63 (s, 1H); 3.07 (m, 2H); 2.49 (m, 1H); 2.38 (m, 1H)
1-121: ¹H-NMR (400.0 MHz, CDCl₃):
δ = 8.52 (s, 2H); 7.29 (m, 1H); 7.25-7.17 (m, 3H); 4.33 (s, 2H); 2.99 (m, 2H); 2.35 (m, 2H)
1-122: ¹H-NMR (400.0 MHz, CDCl₃):
δ = 8.54 (dd, 1H); 7.70 (dd, 1H); 7.48 (dd, 1H); 7.37-7.30 (m, 3H); 7.24 (dd, 1H); 5.47 (s, 1H); 2.95 (m, 2H); 2.34 (m, 2H)
1-123: ¹H-NMR (400.0 MHz, CDCl₃):
δ = 8.50 (d, 1H); 7.54 (dd, 1H); 7.25-7.17 (m, 4H); 6.97 (d, 1H); 4.17 (s, 2H); 2.87 (m, 2H); 2.26 (m, 2H)
1-124: ¹H-NMR (400.0 MHz, CDCl₃):
δ = 8.52 (s, 2H); 7.91 (s, 1H); 7.90 (m, 1H); 7.83 (dd, 1H); 7.57 (ddd, 1H); 7.53 (ddd, 1H); 7.38 (dd, 1H); 6.06 (s, 1H)
1-125: ¹H-NMR (400.0 MHz, CDCl₃):
δ = 8.65 (d, 1H); 7.94 (s, 1H); 7.90-7.88 (m, 2H); 7.79 (m, 1H); 7.62-7.54 (m, 2H); 7.36 (dd, 1H); 6.26 (s, 1H)
1-126: ¹H-NMR (400.0 MHz, CDCl₃):
δ = 8.40 (dd, 1H); 7.94-7.90 (m, 2H); 7.69 (m, 1H); 7.63-7.50 (m, 2H); 7.39 (m, 1H); 7.32 (dd, 1H); 6.13 (s, 1H)
1-127: ¹H-NMR (400.0 MHz, CDCl₃):
δ = 8.30 (dd, 1H); 7.34-7.27 (m, 3H); 7.22-7.12 (m, 4H); 7.02 (m, 1H); 4.10 (d, 2H)
1-128: ¹H-NMR (400.0 MHz, CDCl₃):
δ = 8.52 (s, 2H); 8.15 (dd, 1H); 8.00 (s, 1H); 7.93 (m, 1H); 7.88 (dd, 1H); 7.75 (dd, 1H); 5.76 (s, 1H)
1-129: ¹H-NMR (400.0 MHz, CDCl₃):
δ = 8.45 (s, 2H); 7.99 (m, 1H); 7.84 (s, 1H); 7.46-7.36 (m, 4H); 4.31 (d, 2H)
1-130: ¹H-NMR (400.0 MHz, CDCl₃):
δ = 8.24 (dd, 1H); 7.87 (m, 1H); 7.86 (s, 1H); 7.46-7.32 (m, 5H); 4.15 (d, 2H)
1-131: ¹H-NMR (400.0 MHz, CDCl₃):
δ = 8.46 (s, 2H); 7.97 (m, 1H); 7.95 (s, 1H); 7.75 (dd, 1H); 7.73 (dd, 1H); 7.59 (dd, 1H); 4.22 (s, 2H)
1-132: ¹H-NMR (400.0 MHz, CDCl₃):
δ = 8.68 (s, 2H); 7.84 (dd, 1H); 7.67 (dd, 1H); 7.59 (dt, 1H); 7.52 (ddd, 1H); 6.95 (s, 1H); 6.83 (t, 1H); 5.95 (s, 1H)
1-133: ¹H-NMR (400.0 MHz, CDCl₃):
δ = 8.60 (s, 2H); 7.70 (dd, 1H); 7.48-7.39 (m, 3H); 6.87 (s, 1H); 6.79 (t, 1H); 4.50 (s, 2H)
1-134: ¹H-NMR (400.0 MHz, CDCl₃):
δ = 8.59 (s, 2H); 7.84 (dd, 1H); 7.67 (dd, 1H); 7.59 (dt, 1H); 7.52 (ddd, 1H); 6.94 (s, 1H); 6.83 (t, 1H); 5.97 (s, 1H)
1-135: ¹H-NMR (400.0 MHz, CDCl₃):
δ = 8.51 (s, 2H); 7.70 (d, 1H); 7.48-7.38 (m, 3H); 6.87 (s, 1H); 6.79 (t, 1H); 4.52 (s, 2H)
1-136: ¹H-NMR (400.0 MHz, CDCl₃):
δ = 8.57 (s, 2H); 7.82 (dd, 1H); 7.55-7.51 (m, 2H); 7.43 (m, 1H); 7.37 (m, 1H); 6.99 (m, 1H); 6.55 (d, 1H); 6.21 (s, 1H)
1-137: ¹H-NMR (400.0 MHz, CDCl₃):
δ = 8.49 (s, 2H); 7.49 (d, 1H); 7.44-7.42 (m, 2H); 7.35-7.31 (m, 2H); 6.94 (dd, 1H); 6.53 (d, 1H); 4.48 (s, 2H)
1-138: ¹H-NMR (400.0 MHz, CDCl₃):
δ = 8.60 (s, 2H); 7.95 (s, 1H); 7.82 (d, 1H); 7.78 (dd, 1H); 7.70 (s, 1H); 7.49 (dd, 1H); 5.42 (s, 1H)
1-139: ¹H-NMR (400.0 MHz, CDCl₃):
δ = 10.50 (bs, 1H); 8.75 (s, 2H); 7.90 (d, 1H); 7.81 (dd, 1H); 7.68 (m, 1H); 7.62 (m, 1H); 6.25 (m, 1H); 5.91 (t, 1H)
1-140: ¹H-NMR (400.0 MHz, CDCl₃):
δ = 8.55 (s, 2H); 7.45 (dd, 1H); 7.42 (dd, 1H); 7.34 (dd, 3H); 6.81 (t, 1H); 6.57 (s, 1H); 4.22 (s, 2H)
1-141: ¹H-NMR (400.0 MHz, CDCl₃):
δ = 8.55 (s, 2H); 7.92 (d, 1H); 7.62-7.59 (m, 2H); 7.51-7.44 (m, 2H); 7.31 (d, 1H); 7.11-7.07 (m, 2H); 4.53 (d, 1H); 4.46 (d, 1H)
1-142: ¹H-NMR (400.0 MHz, CDCl₃):
δ = 8.46 (s, 2H); 8.26 (dd, 1H); 7.83-7.80 (m, 2H); 7.59 (m, 1H); 7.51 (m, 1H); 7.27 (d, 1H); 7.09-7.05 (m, 2H); 4.66 (s, 2H)
1-143: ¹H-NMR (400.0 MHz, CDCl₃):
δ = 8.47 (s, 2H); 7.92 (dd, 1H); 7.63-7.59 (m, 2H); 7.51-7.43 (m, 2H); 7.31 (m, 1H); 7.11-7.06 (m, 2H); 4.53 (d, 1H); 4.48 (d, 1H)
1-144: ¹H-NMR (400.0 MHz, CDCl₃):
δ = 8.38 (s, 2H); 8.26 (dd, 1H); 7.85-7.81 (m, 2H); 7.58 (m, 1H); 7.50 (m, 1H); 7.28 (d, 1H); 7.10-7.05 (m, 2H); 4.67 (s, 2H)
1-145: ¹H-NMR (400.0 MHz, CDCl₃):
δ = 8.60 (s, 2H); 7.92 (d, 1H); 7.56 (dd, 1H); 7.44-7.33 (m, 5H); 4.88 (d, 1H); 4.50 (d, 1H)
1-146: ¹H-NMR (400.0 MHz, CDCl₃):
δ = 8.50 (s, 2H); 7.81 (dd, 1H); 7.54-7.50 (m, 2H); 7.42 (m, 1H); 7.38 (m, 1H); 7.00 (dd, 1H); 6.60 (d, 1H); 6.24 (s, 1H)
1-147: ¹H-NMR (400.0 MHz, CDCl₃):
δ = 8.42 (d, 2H); 7.48 (dd, 1H); 7.43-7.40 (m, 2H); 7.34-7.30 (m, 2H); 6.95 (dd, 1H); 6.57 (d, 1H); 4.50 (s, 2H)
1-148: ¹H-NMR (400.0 MHz, CDCl₃):
δ = 8.51 (s, 2H); 7.94 (d, 1H); 7.55-7.47 (m, 2H); 7.49 (m, 5H); 4.91 (d, 1H); 4.53 (d, 1H)
1-149: ¹H-NMR (400.0 MHz, CDCl₃):
δ = 8.50 (s, 2H); 7.97 (dd, 1H); 7.33-7.24 (m, 2H); 7.41 (d, 1H); 4.47 (d, 1H); 4.38 (d, 1H); 3.36 (m, 1H); 3.13 (m, 1H); 2.71 (m, 1H);
2.40 (m, 1H)
1-150: ¹H-NMR (400.0 MHz, CDCl₃):
δ = 8.49 (s, 2H); 8.06 (d, 1H); 7.60 (dd, 1H); 7.53 (d, 1H); 7.47 (dd, 1H); 4.89 (s, 2H); 3.79 (m, 2H); 2.55 (m, 2H)
1-151: ¹H-NMR (400.0 MHz, CDCl₃):
δ = 8.48 (s, 2H); 8.42 (s, 2H); 7.24-7.20 (m, 2H); 7.11 (m, 1H); 4.32 (s, 2H)
1-152: ¹H-NMR (400.0 MHz, CDCl₃):
δ = 8.48 (s, 2H); 8.35 (s, 2H); 7.24-7.19 (m, 2H); 7.11 (m, 1H); 4.34 (s, 2H)
1-153: ¹H-NMR (400.0 MHz, CDCl₃):
δ = 8.40 (s, 2H); 8.35 (s, 2H); 7.24-7.20 (m, 2H); 7.11 (m, 1H); 4.32 (s, 2H)
1-154: ¹H-NMR (400.0 MHz, CDCl₃):
δ = 8.43 (d, 2H); 8.39 (s, 2H); 7.24-7.19 (m, 2H); 7.11 (m, 1H); 4.33 (s, 2H)
1-155: ¹H-NMR (400.0 MHz, CDCl₃):
δ = 8.49 (s, 2H); 8.33 (s, 2H); 7.40 (dd, 1H); 7.35 (dd, 1H); 7.23 (dd, 1H); 4.28 (s, 2H)
1-156: ¹H-NMR (400.0 MHz, CDCl₃):
δ = 8.41 (s, 2H); 8.34 (s, 2H); 7.40 (dd, 1H); 7.35 (dd, 1H); 7.22 (dd, 1H); 4.29 (s, 2H)
1-157: ¹H-NMR (400.0 MHz, CDCl₃):
δ = 8.41 (s, 2H); 8.40 (s, 2H); 7.40 (dd, 1H); 7.35 (dd, 1H); 7.23 (dd, 1H); 4.29 (s, 2H)
1-158: ¹H-NMR (400.0 MHz, CDCl₃):
δ = 8.70 (s, 1H); 8.56 (s, 2H); 8.09 (dd, 1H); 7.50 (m, 1H); 7.45 (m, 1H); 7.38 (m, 1H); 4.80 (s, 2H)
1-159: ¹H-NMR (400.0 MHz, CDCl₃):
δ = 8.57 (s, 2H); 8.03 (dd, 1H); 7.47 (m, 1H); 7.41 (m, 1H); 7.34 (m, 1H); 4.78 (s, 2H); 2.61 (s, 3H)
1-160: ¹H-NMR (400.0 MHz, CDCl₃):
δ = 8.55 (s, 2H); 8.09 (dd, 1H); 7.52 (m, 1H); 7.45 (m, 1H); 7.39 (m, 1H); 6.83 (t, 1H); 4.78 (s, 2H)
1-161: ¹H-NMR (400.0 MHz, CDCl₃):
δ = 8.58 (s, 2H); 8.11 (dd, 1H); 7.54 (m, 1H); 7.47 (m, 1H); 7.40 (m, 1H); 4.78 (s, 2H)
1-162: ¹H-NMR (400.0 MHz, CDCl₃):
δ = 8.39 (s, 2H); 8.32 (s, 2H); 7.26 (d, 1H); 7.20-7.19 (m, 2H); 4.22 (s, 2H); 2.10 (s, 3H)
1-163: ¹H-NMR (400.0 MHz, CDCl₃):
δ = 8.35 (dd, 1H); 8.33 (s, 2H); 8.21 (d, 1H); 7.61 (ddd, 1H); 7.52 (ddd, 1H); 7.33-7.27 (m, 3H); 4.56 (s, 2H)
1-164: ¹H-NMR (400.0 MHz, CDCl₃):
δ = 8.41 (s, 2H); 8.36 (dd, 1H); 8.19 (d, 1H); 7.62 (ddd, 1H); 7.52 (ddd, 1H); 7.32-7.28 (m, 3H); 4.55 (s, 2H)
1-165: ¹H-NMR (400.0 MHz, CDCl₃):
δ = 8.48 (s, 2H); 8.32 (s, 2H); 7.27 (m, 1H); 7.20-7.19 (m, 2H); 4.21 (s, 2H); 2.10 (s, 3H)
1-166: ¹H-NMR (400.0 MHz, CDCl₃):
δ = 8.47 (s, 2H); 8.39 (s, 2H); 7.27 (m, 1H); 7.21-7.20 (m, 2H); 4.20 (s, 2H); 2.10 (s, 3H)
1-167: ¹H-NMR (400.0 MHz, CDCl₃):
δ = 8.53 (s, 2H); 8.36 (s, 2H); 7.66 (dd, 1H); 7.47-7.45 (m, 2H); 7.36 (ddd, 1H); 4.50 (s, 2H)
1-168: ¹H-NMR (400.0 MHz, CDCl₃):
δ = 8.61 (s, 2H); 7.41 (ddd, 1H); 7.34-7.19 (m, 3H); 4.50 (s, 2H); 2.93 (t, 2H); 2.20 (m, 2H); 1.84 (m, 2H)
1-169: ¹H-NMR (400.0 MHz, CDCl₃):
δ = 8.69 (s, 2H); 8.61 (s, 2H); 8.02 (dd, 1H); 7.43-7.40 (m, 2H); 7.30 (dd, 1H); 5.02 (d, 1H); 4.53 (d, 1H)
1-170: ¹H-NMR (400.0 MHz, CDCl₃):
δ = 8.76 (s, 2H); 8.42 (s, 2H); 8.37 (dd, 1H); 7.62 (ddd, 1H); 7.53 (m, 1H); 7.38 (dd, 1H); 4.77 (s, 2H)
1-171: ¹H-NMR (400.0 MHz, CDCl₃):
δ = 8.49 (s, 2H); 8.34 (s, 2H); 7.43 (dd, 1H); 7.35 (ddd, 1H); 7.27 (ddd, 1H); 7.13 (dd, 1H); 4.27 (s, 2H)
1-180: ¹H-NMR (300.0 MHz, d₆-DMSO):
δ = 8.93 (d, 2H); 8.44 (s, 1H); 7.90 (s, 1H); 7.52-7.33 (m, 3H); 7.13 (m, 1H); 6.53 (s, 1H); 5.70 (d, H); 5.59 (d, 1H)
1-182: ¹H-NMR (300.0 MHz, d₆-DMSO):
δ = 8.95 (s, 2H); 8.44 (s, 1H); 7.88 (s, 1H); 7.52-7.33 (m, 3H); 7.14 (m, 1H); 6.52 (s, 1H); 5.69 (d, H); 5.58 (d, 1H)
1-184: ¹H-NMR (400.0 MHz, CDCl₃):
δ = 8.61 (s, 2H); 7.19-7.16 (m, 3H); 4.43 (s, 2H); 3.76-3.74 (t, 2H); 3.70-3.66 (t, 2H); 2.52-2.40 (m, 2H); 2.31 (s, 3H)
1-185: ¹H-NMR (400.0 MHz, CDCl₃):
δ = 8.53 (s, 2H); 7.25-7.20 (m, 1H); 7.14-7.09 (m, 2H); 4.48 (s, 2H); 3.75-3.72 (t, 2H); 3.67-3.63 (t, 2H); 2.53-2.46 (m, 2H)
1-186: ¹H-NMR (400.0 MHz, CDCl₃):
δ = 8.71 (s, 2H); 7.25-7.21 (m, 1H); 7.14-7.09 (m, 2H); 4.44 (s, 2H); 3.56-3.52 (m, 2H); 2.41-2.27 (m, 4H)
1-187: ¹H-NMR (400.0 MHz, CDCl₃):
δ = 8.71 (s, 2H); 7.32-7.24 (m, 1H); 7.22-7.17 (m, 2H); 4.42 (s, 2H); 3.71-3.61 (m, 2H); 2.89-2.57 (m, 2H)
1-188: ¹H-NMR (400.0 MHz, CDCl₃):
δ = 8.60 (s, 2H); 7.20-7.16 (m, 3H); 4.42 (s, 2H); 3.56 (t, 2H); 2.43-2.26 (m, 7H)
1-189: ¹H-NMR (400.0 MHz, CDCl₃):
δ = 8.61 (s, 2H); 7.24-7.21 (m, 1H); 7.19-7.08 (m, 2H); 4.39 (s, 2H); 3.80-3.71 (m, 2H); 2.93-2.83 (m, 2H); 2.39 (S, 3H)
1-190: ¹H-NMR (400.0 MHz, CDCl₃):
δ = 8.62 (s, 2H); 7.39-7.37 (m, 1H); 7.25-7.20 (m, 2H); 4.51 (s, 2H); 3.77-3.73 (m, 4H); 2.55-2.49 (m, 2H)
1-191: ¹H-NMR (400.0 MHz, CDCl₃):
δ = 8.53 (s, 2H); 7.39-7.36 (m, 1H); 7.25-7.17 (m, 2H); 4.53 (s, 2H); 3.77-3.73 (m, 4H); 2.56-2.49 (m, 2H)
1-192: ¹H-NMR (400.0 MHz, CDCl₃):
δ = 8.71 (s, 2H); 7.24-7.20 (m, 1H); 7.14-7.09 (m, 2H); 4.44 (s, 2H); 3.75-3.72 (t, 2H); 3.67-3.63 (t, 2H); 2.53-2.46 (m, 2H)
1-193: ¹H-NMR (400.0 MHz, CDCl₃):
δ = 8.62 (s, 2H); 7.25-7.20 (m, 1H); 7.14-7.09 (m, 2H); 4.47 (s, 2H); 3.75-3.72 (t, 2H); 3.67-3.63 (t, 2H); 2.53-2.46 (m, 2H)
1-194: ¹H-NMR (400.0 MHz, CDCl₃):
δ = 8.38 (s, 2H); 8.19 (s, 1H); 7.35-7.29 (m, 1H); 7.21-7.17 (m, 1H); 7.10-7.04 (m, 2H); 6.96-6.95 (m, 1H), 4.15 (s, 2H)
1-195: ¹H-NMR (400.0 MHz, CDCl₃):
δ = 8.61 (s, 2H); 7.05-6.95 (m, 3H); 4.31 (s, 2H); 4.08 (t, 2H); 2.29 (m, 2H); 1.92 (m, 2H)
1-196: ¹H-NMR (400.0 MHz, CDCl₃):
δ = 8.60 (s, 2H); 7.98 (dd, 1H); 7.51 (ddd, 1H); 7.45 (ddd, 1H); 7.38 (dd, 1H); 4.46 (d, 1H); 4.35 (d, 1H); 3.12 (m, 1H); 2.91 (m, 1H);
2.31-1.97 (m, 4H)
1-197: ¹H-NMR (400.0 MHz, CDCl₃):
δ = 8.60 (s, 2H); 8.07 (ddd, 1H); 7.58 (ddd; 1H); 7.48-7.44 (m, 2H); 4.85 (s, 2H); 3.51 (t, 2H); 2.27 (m, 2H); 2.03 (m, 2H)
1-198: ¹H-NMR (400.0 MHz, CDCl₃):
δ = 8.62 (s, 2H); 7.42 (dd, 1H); 7.28-7.18 (m, 3H); 4.50 (s, 2H); 3.61 (t, 2H); 3.03 (t, 2H); 2.03 (quint, 2H)
1-199: ¹H-NMR (400.0 MHz, CDCl₃):
δ = 8.61 (s, 2H); 7.99 (dd, 1H); 7.51 (ddd, 1H); 7.45 (ddd, 1H); 7.37 (dd, 1H); 4.49 (d, 1H); 4.37 (d, 1H); 3.72 (m, 1H); 3.62 (m, 1H);
3.27 (m, 1H); 2.93 (m, 1H); 2.36 (m, 1H); 2.22 (m, 1H)
1-200: ¹H-NMR (400.0 MHz, CDCl₃):
δ = 8.61 (s, 2H); 8.07 (ddd, 1H); 7.58 (ddd; 1H); 7.48-7.44 (m, 2H); 4.86 (s, 2H); 3.65-3.57 (m, 4H); 2.23 (m, 2H)
1-201: ¹H-NMR (400.0 MHz, CDCl₃):
δ = 8.53 (s, 2H); 8.32 (d, 2H); 7.67 (dd, 1H); 7.48-7.43 (m, 2H); 7.35 (ddd, 1H); 4.51 (s, 2H)
1-202: ¹H-NMR (400.0 MHz, CDCl₃):
δ = 8.46 (s, 2H); 8.32 (d, 2H); 7.67 (dd, 1H); 7.48-7.41 (m, 2H); 7.35 (ddd, 1H); 4.52 (s, 2H)
1-203: ¹H-NMR (400.0 MHz, CDCl₃):
δ = 8.50 (s, 2H); 7.98 (dd, 1H); 7.51 (ddd, 1H); 7.45 (ddd, 1H); 7.38 (dd, 1H); 4.48 (d, 1H); 4.38 (d, 1H); 3.12 (m, 1H); 2.89 (m, 1H);
2.31-1.97 (m, 4H)
1-204: ¹H-NMR (400.0 MHz, CDCl₃):
δ = 8.51 (s, 2H); 8.07 (ddd, 1H); 7.58 (ddd; 1H); 7.48-7.44 (m, 2H); 4.86 (s, 2H); 3.51 (t, 2H); 2.27 (m, 2H); 2.03 (m, 2H)
1-205: ¹H-NMR (400.0 MHz, CDCl₃):
δ = 8.52 (s, 2H); 7.99 (dd, 1H); 7.51 (ddd, 1H); 7.45 (ddd, 1H); 7.37 (dd, 1H); 4.50 (dd, 1H); 4.39 (d, 1H); 3.72 (m, 1H); 3.62 (m, 1H);
3.27 (m, 1H); 2.93 (m, 1H); 2.36 (m, 1H); 2.22 (m, 1H)
1-206: ¹H-NMR (400.0 MHz, CDCl₃):
δ = 8.52 (s, 2H); 8.07 (dd, 1H); 7.58 (ddd; 1H); 7.48-7.43 (m, 2H); 4.88 (s, 2H); 3.65-3.57 (m, 4H); 2.23 (m, 2H)
1-207: ¹H-NMR (400.0 MHz, CDCl₃):
δ = 8.51 (s, 2H); 7.95 (s, 1H); 7.83 (dd, 1H); 7.78 (dd, 1H); 7.68 (s, 1H); 7.49 (dd, 1H); 5.45 (s, 1H)
1-208: ¹H-NMR (400.0 MHz, CDCl₃):
δ = 8.53 (s, 2H); 7.89 (s, 1H); 7.67 (m, 1H); 7.65 (dd, 1H); 7.41 (dd, 1H); 7.35 (dd, 1H); 4.12 (s, 2H)
1-209: ¹H-NMR (400.0 MHz, CDCl₃):
δ = 8.44 (s, 2H); 7.89 (s, 1H); 7.65 (s, 1H); 7.64 (dd, 1H); 7.41 (dd, 1H); 7.35 (dd, 1H); 4.13 (s, 2H)
2-1: ¹H-NMR (400.0 MHz, CDCl₃):
δ = 8.46 (s, 2H); 7.06 (s, 1H); 6.77 (t, 1H); 6.46 (s, 1H); 4.44 (s, 2H); 2.56 (s, 3H); 2.20 (s, 3H)
2-2: ¹H-NMR (400.0 MHz, CDCl₃):
δ = 8.56 (s, 2H); 7.06 (s, 1H); 6.78 (t, 1H); 6.49 (s, 1H); 4.42 (s, 2H); 2.55 (s, 3H); 2.20 (s, 3H)
2-5: ¹H-NMR (400.0 MHz, CDCl₃):
δ = 8.56 (s, 2H); 7.15 (s, 1H); 6.82 (t, 1H); 6.71 (s, 1H); 6.60 (s, 1H); 2.56 (s, 3H); 2.22 (s, 3H)
2-6: ¹H-NMR (400.0 MHz, CDCl₃):
δ = 8.65 (s, 2H); 7.17 (s, 1H); 6.84 (t, 1H); 6.73 (s, 1H); 6.59 (s, 1H); 2.56 (s, 3H); 2.23 (s, 3H)
2-7: ¹H-NMR (400.0 MHz, CDCl₃):
δ = 8.42 (s, 2H); 8.41 (s, 2H); 7.38 (d, 1H); 7.16 (d, 1H); 4.50 (s, 2H); 2.58 (s, 3H)
2-8: ¹H-NMR (400.0 MHz, CDCl₃):
δ = 8.41 (s, 2H); 8.35 (s, 2H); 7.38 (d, 1H); 7.15 (d, 1H); 4.50 (s, 2H); 2.57 (s, 3H)
2-9: ¹H-NMR (400.0 MHz, CDCl₃):
δ = 8.50 (s, 2H); 7.02 (s, 2H); 4.53 (d, 2H); 3.91 (t, 2H); 2.49 (s, 3H); 2.05 (m, 2H); 1.87 (m, 2H)
2-10: ¹H-NMR (400.0 MHz, CDCl₃):
δ = 8.51 (s, 2H); 7.58 (d, 1H); 7.12 (d, 1H); 4.58 (d, 2H); 3.43 (t, 2H); 2.55 (s, 3H); 2.37-2.22 (m, 4H)
2-11: ¹H-NMR (400.0 MHz, CDCl₃):
δ = 8.60 (s, 2H); 7.58 (d, 1H); 7.12 (d, 1H); 4.56 (d, 2H); 3.43 (t, 2H); 2.55 (s, 3H); 2.37-2.22 (m, 4H)
2-12: ¹H-NMR (400.0 MHz, CDCl₃):
δ = 8.49 (s, 2H); 8.34 (s, 2H); 7.39 (d, 1H); 7.16 (d, 1H); 4.49 (s, 2H); 2.57 (s, 3H)
2-13: ¹H-NMR (400.0 MHz, CDCl₃):
δ = 8.48 (s, 2H); 8.45 (s, 2H); 7.94 (d, 1H); 7.52 (d, 1H); 4.76 (s, 2H); 2.85 (s, 3H)
2-14: ¹H-NMR (400.0 MHz, CDCl₃):
δ = 8.56 (s, 2H); 7.61 (d, 1H); 7.46 (d, 1H); 4.81 (s, 2H); 4.08 (t, 2H); 2.78 (s, 3H); 2.04 (m, 2H); 1.96 (m, 2H)

The present invention further provides for the use of one or more compounds of the general formula (I) and/or salts thereof, as defined above, preferably in one of the embodiments identified as preferred or particularly preferred, in particular one or more compounds of the formulae (1-1) to (1-209), (2-1) to (2-14) and/or salts thereof, in each case as defined above, as herbicide and/or plant growth regulator, preferably in crops of useful plants and/or ornamentals.

The present invention further provides a method of controlling harmful plants and/or for regulating the growth of plants, characterised in that an effective amount of
one or more compounds of the general formula (I) and/or salts thereof, as defined above, preferably in one of the embodiments identified as preferred or particularly preferred, in particular one or more compounds of the formulae (1-1) to (1-209) or (2-1) to (2-14) and/or salts thereof, in each case as defined above, or
a composition of the invention, as defined below, is applied to the (harmful) plants, seeds of (harmful) plants, the soil in which or on which the (harmful) plants grow or the area under cultivation.

The present invention also provides a method for controlling unwanted plants, preferably in crops of useful plants, characterised in that an effective amount of
one or more compounds of the general formula (I) and/or salts thereof, as defined above, preferably in one of the embodiments identified as preferred or particularly preferred, in particular one or more compounds of the formulae (1-1) to (1-209) or (2-1) to (2-14) and/or salts thereof, in each case as defined above, or
a composition of the invention, as defined below,
is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop-growing land or non-crop-growing land) or the area under cultivation (i.e. the area on which the unwanted plants will grow).

The present invention also further provides a method for controlling harmful plants or for regulating the growth of plants, preferably of useful plants, characterised in that an effective amount of
one or more compounds of the general formula (I) and/or salts thereof, as defined above, preferably in one of the embodiments identified as preferred or particularly preferred, in particular one or more compounds of the formulae (1-1) to (1-209) or (2-1) to (2-14) and/or salts thereof, in each case as defined above, or
a composition of the invention, as defined below,
is applied to the plant, the seed of the plant (i.e. plant seed, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the inventive compounds or the inventive compositions can be applied for example by pre-sowing (if appropriate also by incorporation into the soil), pre-emergence and/or post-emergence processes. Specific examples of some representatives of the monocotyledonous and dicotyledonous weed flora which can be controlled by the inventive compounds are as follows, though there is no intention to restrict the enumeration to particular species.

In a method of the invention for controlling harmful plants or for regulating the growth of plants, preference is given to using one or more compounds of the general formula (I) and/or salts thereof for control of harmful plants or for regulation of growth in crops of useful plants or ornamental plants, where the useful plants or ornamental plants in a preferred configuration are transgenic plants.

The inventive compounds of the general formula (I) and/or salts thereof are suitable for controlling the following genera of monocotyledonous and dicotyledonous harmful plants:

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the inventive compounds of the general formula (I) are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

If the active ingredients of the general formula (I) are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the inventive compounds of the general formula (I) display outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, are damaged only to an insignificant extent, or not at all, depending on the structure of the respective compound according to the invention and its application rate. For these reasons, the present compounds are very suitable for selective control of unwanted plant growth in plant crops such as agriculturally useful plants or ornamental plants.

In addition, the inventive compounds of the general formula (I) (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the compounds of the general formula (I) can also be used to control harmful plants in crops of genetically modified plants or plants modified by conventional mutagenesis. In general, the transgenic plants are characterised by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the inventive compounds of the general formula (I) and/or salts thereof in economically important transgenic crops of useful plants and ornamentals, for example of cereals such as wheat, barley, rye, oats, millet, rice and maize or else crops of sugar beet, cotton, soybean, oilseed rape, potato, tomato, peas and other vegetables.

It is preferable to employ the inventive compounds of the general formula (I) also as herbicides in crops of useful plants which are resistant, or have been made resistant by recombinant means, to the phytotoxic effects of the herbicides.

By virtue of their herbicidal and plant growth regulatory properties, the inventive compounds of the general formula (I) can also be used to control harmful plants in crops of genetically modified plants which are known or are yet to be developed. In general, the transgenic plants are characterised by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the inventive compounds of the general formula (I) or salts thereof in economically important transgenic crops of useful plants and ornamentals, for example of cereals such as wheat, barley, rye, oats, triticale, millet, rice, cassava and maize, or else crops of sugar beet, cotton, soybean, oilseed rape, potatoes, tomatoes, peas and other vegetables.

It is preferable to employ the compounds of the general formula (I) as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical.

When expressing nucleic acid molecules in plants, the protein synthesised may be localised in any desired compartment of the plant cell. However, to achieve localisation in a particular compartment, it is possible, for example, to join the coding region to DNA sequences which ensure localisation in a particular compartment. Sequences of this kind are known to the person skilled in the art (see, for example, Braun et al., EMBO J. 11 (1992), 3219-3227). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Obtainable in this way are transgenic plants having properties altered by overexpression, suppression or inhibition of homologous (=natural) genes or gene sequences or expression of heterologous (=foreign) genes or gene sequences.

It is preferable to employ the inventive compounds of the general formula (I) in transgenic crops which are resistant to growth regulators, for example dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, glyphosates, glufosinates or benzoylisoxazoles and analogous active ingredients.

When the inventive compounds of the general formula (I) are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the general formula (I) and/or salts thereof as herbicides for controlling harmful plants in crops of useful plants or ornamentals, optionally in transgenic crop plants.

Preference is given to the use of compounds of the general formula (I) in cereals, here preferably maize, wheat, barley, rye, oats, millet or rice, by the pre- or post-emergence method.

Preference is also given to the use of compounds of the general formula (I) in soybean by the pre-emergence or post-emergence method.

The use of inventive compounds of the formula (I) for the control of harmful plants or for growth regulation of plants also includes the case in which a compound of the general formula (I) or its salt is not formed from a precursor substance (“prodrug”) until after application on the plant, in the plant or in the soil.

The invention also provides the use of one or more compounds of the general formula (I) or salts thereof or of a composition according to the invention (as defined below) (in a method) for controlling harmful plants or for regulating the growth of plants which comprises applying an effective amount of one or more compounds of the general formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterised in that the composition comprises
(a) one or more compounds of the general formula (I) and/or salts thereof, as defined above, preferably in one of the embodiments identified as preferred or particularly preferred, in particular one or more compounds of the formulae (1-1) to (1-209) or (2-1) to (2-14) and/or salts thereof, in each case as defined above,
and
(b) one or more further substances selected from groups (i) and/or (ii):
(i) one or more further agrochemically active substances, preferably selected from the group consisting of insecticides, acaricides, nematicides, further herbicides (i.e. those not conforming to the general formula (I) defined above), fungicides, safeners, fertilisers and/or further growth regulators,
(ii) one or more formulation auxiliaries customary in crop protection.

The further agrochemically active substances of component (i) of a composition of the invention are preferably selected from the group of substances mentioned in “The Pesticide Manual”, 16th edition, The British Crop Protection Council and the Royal Soc. of Chemistry, 2012.

A herbicidal or plant growth-regulating composition of the invention comprises preferably one, two, three or more formulation auxiliaries (ii) customary in crop protection selected from the group consisting of surfactants, emulsifiers, dispersants, film formers, thickeners, inorganic salts, dusting agents, carriers that are solid at 25° C. and 1013 mbar, preferably adsorptive granulated inert materials, wetting agents, antioxidants, stabilisers, buffer substances, antifoam agents, water, organic solvents, preferably organic solvents miscible with water in any ratio at 25° C. and 1013 mbar.

The inventive compounds of the general formula (I) can be used in the form of wettable powders, emulsifiable concentrates, sprayable solutions, dusting products or granules in the customary formulations. The invention therefore also provides herbicidal and plant growth-regulating compositions which comprise compounds of the general formula (I) and/or salts thereof.

The inventive compounds of the general formula (I) and/or salts thereof can be formulated in various ways according to which biological and/or physicochemical parameters are specified. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation auxiliaries, such as inert materials, surfactants, solvents and further additives, are known to the person skilled in the art and are described, for example, in: Watkins, “Handbook of Insecticide Dust Diluents and Carriers”, 2nd ed., Darland Books, Caldwell N. J., H.v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesellschaft, Stuttgart 1976, Winnacker-Küichler, “Chemische Technologie”, Volume 7, C. Hanser Verlag Munich, 4th ed. 1986.

Wettable powders are preparations which can be dispersed uniformly in water and, in addition to the active ingredient, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts, for example calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They can be produced, for example, by wet grinding by means of standard commercial bead mills and optionally the addition of surfactants, as have already been listed e.g. above for the other types of formulation.

Emulsions, e.g. oil-in-water emulsions (EW), can be prepared, for example, by means of stirrers, colloid mills and/or static mixers using aqueous organic solvents and optionally surfactants, as have already been listed e.g. above for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertiliser granules—if desired as a mixture with fertilisers.

Water-dispersible granules are produced generally by the customary processes such as spray-drying, fluidised-bed granulation, pan granulation, mixing with high-speed mixers and extrusion without solid inert material.

For the production of pan granules, fluidised bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection agents, see, for example, G. C. Klingman, “Weed Control as a Science”, John Wiley and Sons, Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical preparations, preferably herbicidal or plant growth-regulating compositions, of the present invention preferably comprise a total amount of 0.1 to 99% by weight, preferably 0.5 to 95% by weight, more preferably 1 to 90% by weight, especially preferably 2 to 80% by weight, of active ingredients of the general formula (I) and salts thereof.

In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partly on whether the active ingredient is in liquid or solid form and on which granulation auxiliaries, fillers, and so forth are used. In the water-dispersible granules, the content of active ingredient is, for example, between 1% and 95% by weight, preferably between 10% and 80% by weight.

In addition, the active ingredient formulations mentioned optionally comprise the respectively customary stickers, wetters, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described inter alia in “Chemistry and Technology of Agrochemical Formulations”, ed. D. A. Knowles, Kluwer Academic Publishers (1998).

The inventive compounds of the general formula (I) or salts thereof can be used as such or in the form of their preparations (formulations) in a combination with other pesticidally active substances, for example insecticides, acaricides, nematicides, herbicides, fungicides, safeners, fertilisers and/or growth regulators, for example in the form of a finished formulation or of a tankmix. The combination formulations can be produced on the basis of the abovementioned formulations, taking account of the physical properties and stabilities of the active ingredients to be combined.

Combination partners usable for the inventive compounds of the general formula (I) in mixed formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012, and the literature cited therein.

Of particular interest is the selective control of harmful plants in crops of useful plants and ornamentals. Although the inventive compounds of the general formula (I) have already demonstrated very good to adequate selectivity in a large number of crops, in principle, in some crops and in particular also in the case of mixtures with other, less selective herbicides, phytotoxicities on the crop plants may occur. In this connection, combinations of compounds of the general formula (I) according to the invention are of particular interest which comprise the compounds of the general formula (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, maize, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20. Analogously to the compounds of the general formula (I) or mixtures thereof, the safeners can be formulated with further herbicides/pesticides and be provided and employed as a finished formulation or tank mix with the herbicides.

For application, the herbicide formulations or herbicide-safener formulations in the commercial form are diluted if appropriate in a customary manner, for example with water in the case of wettable powders, emulsifiable concentrates, dispersions and water-dispersible granules. Preparations in dust form, granules for soil application or granules for scattering and sprayable solutions are not normally diluted further with other inert substances prior to application.

The application rate of the compounds of the general formula (I) and/or their salts is affected to a certain extent by external conditions such as temperature, humidity, etc. The application rate may vary within wide limits. For the application as a herbicide for controlling harmful plants, the total amount of compounds of the general formula (I) and their salts is preferably in the range from 0.001 to 10.0 kg/ha, with preference in the range from 0.005 to 5 kg/ha, more preferably in the range from 0.01 to 1.5 kg/ha, particularly preferably in the range from 0.05 to 1 kg/ha. This applies both to pre-emergence and to post-emergence application.

When the inventive compounds of the general formula (I) and/or salts thereof are used as plant growth regulator, for example as culm stabiliser for crop plants like those mentioned above, preferably cereal plants, such as wheat, barley, rye, triticale, millet, rice or maize, the total application rate is preferably in the range of from 0.001 to 2 kg/ha, preferably in the range of from 0.005 to 1 kg/ha, in particular in the range of from 10 to 500 g/ha, very particularly preferably in the range from 20 to 250 g/ha. This applies both to pre-emergence and to post-emergence application.

The application as culm stabiliser may take place at various stages of the growth of the plants. Preferred is, for example, the application after the tillering phase, at the beginning of the longitudinal growth.

As an alternative, application as plant growth regulator is also possible by treating the seed, which includes various techniques for dressing and coating seed. The application rate depends on the particular techniques and can be determined in preliminary tests.

Combination partners usable for the inventive compounds of the general formula (I) in compositions of the invention (e.g. mixed formulations or in a tankmix) are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as described, for example, from Weed Research 26 (1986) 441-445 or “The Pesticide Manual”, 16th edition, The British Crop Protection Council and the Royal Soc. of Chemistry, 2012, and literature cited therein. Known herbicides or plant growth regulators which can be combined with the compounds of the invention are, for example, the following, where said active ingredients are referred to either by their “common name” in accordance with the International Organization for Standardization (ISO) or by the chemical name or by the code number. They always encompass all the use forms, for example acids, salts, esters and also all isomeric forms such as stereoisomers and optical isomers, even if they are not mentioned explicitly.

Examples of such herbicidal mixing partners are:
acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]-phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. 0-(2,4-dimethyl-6-nitrophenyl)O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

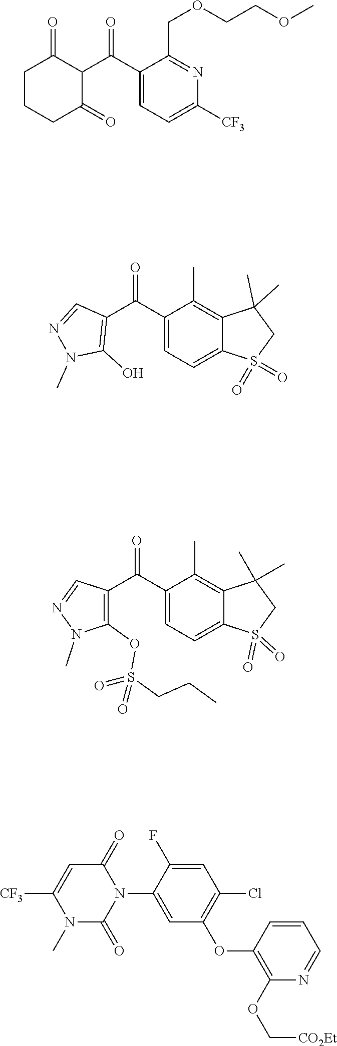 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

Examples of plant growth regulators as possible mixing partners are: acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Useful combination partners for the inventive compounds of the general formula (I) also include, for example, the following safeners:
S1) Compounds from the group of heterocyclic carboxylic acid derivatives:
S1^{a}) Compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333131 and EP-A-269806;
S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;
S1^{d}) Compounds of the triazolecarboxylic acid type (Sid), preferably compounds such as fenchlorazole(-ethyl ester), i.e. ethyl 1-(2,4-dichlorophenyl)-5-trichloromethyl-1H-1,2,4-triazole-3-carboxylate (S1-7), and related compounds, as described in EP-A-174562 and EP-A-346620;
S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13) as described in patent application WO-A-95/07897.
S2) Compounds from the group of the 8-quinolinoxy derivatives (S2):
S2^{a}) Compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5),
methyl (5-chloro-8-quinolinoxy)acetate (S2-6),
allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86750, EP-A-94349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminium, iron, ammonium, quaternary ammonium, sulfonium or phosphonium salts thereof, as described in WO-A-2002/34048;
S2^{b}) Compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Active ingredients of the dichloroacetamide type (S3), which are frequently used as pre-emergence safeners (soil-acting safeners), for example
“dichlormid” (N,N-diallyl-2,2-dichloroacetamide) (S3-1),
“R-29148” (3-dichloroacetyl-2,2,5-trimethyl-1,3-oxazolidine) from Stauffer (S3-2),
“R-28725” (3-dichloroacetyl-2,2-dimethyl-1,3-oxazolidine) from Stauffer (S3-3),
“benoxacor” (4-dichloroacetyl-3,4-dihydro-3-methyl-2H-1,4-benzoxazine) (S3-4),
“PPG-1292” (N-allyl-N-[(1,3-dioxolan-2-yl)methyl]dichloroacetamide) from PPG Industries (S3-5),
“DKA-24” (N-allyl-N-[(allylaminocarbonyl)methyl]dichloroacetamide) from Sagro-Chem (S3-6),
“AD-67” or “MON 4660” (3-dichloroacetyl-1-oxa-3-azaspiro[4.5]decane) from Nitrokemia or Monsanto (S3-7),
“TI-35” (1-dichloroacetylazepane) from TRI-Chemical RT (S3-8),
“diclonon” (dicyclonon) or “BAS145138” or “LAB145138” (S3-9)
((RS)-1-dichloroacetyl-3,3,8a-trimethylperhydropyrrolo[1,2-a]pyrimidin-6-one) from BASF,
“furilazole” or “MON 13900” ((RS)-3-dichloroacetyl-5-(2-furyl)-2,2-dimethyloxazolidine) (S3-10), and the (R) isomer thereof (S3-11).
S4) Compounds from the class of the acylsulfonamides (S4):
S4^{a})N-Acylsulfonamides of the formula (S4^{a}) and salts thereof, as described in WO-A-97/45016,

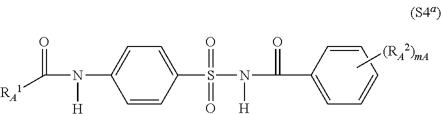

in which
R_{A}¹ is (C₁-C₆)-alkyl, (C₃-C₆)-cycloalkyl, where the 2 latter radicals are substituted by v_{A}substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl;
R_{A}² is halogen, (C₁-C₄)-alkyl, (C₁-C₄)-alkoxy, CF₃;
m_{A} is 1 or 2;
v_{A} is 0, 1, 2 or 3;
S4^{b}) Compounds of the 4-(benzoylsulfamoyl)benzamide type of the formula (S4^{b}) and salts thereof, as described in WO-A-99/16744,

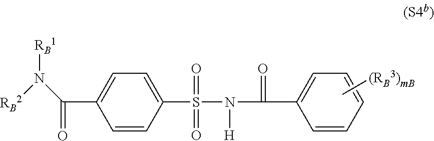

in which
R_{B}¹, R_{B}² are independently hydrogen, (C₁-C₆)-alkyl, (C₃-C₆)-cycloalkyl, (C₃-C₆)-alkenyl, (C₃-C₆)-alkynyl,
R_{B}³ is halogen, (C₁-C₄)-alkyl, (C₁-C₄)-haloalkyl or (C₁-C₄)-alkoxy and
m_{B} is 1 or 2,
e.g. those in which
R_{B}¹=cyclopropyl, R_{B}²=hydrogen and (R_{B}³)=2-OMe (“cyprosulfamide”, S4-1),
R_{B}¹=cyclopropyl, R_{B}²=hydrogen and (R_{B}³)=5-Cl-2-OMe (S4-2),
R_{B}¹=ethyl, R_{B}²=hydrogen and (R_{B}³)=2-OMe (S4-3),
R_{B}¹=isopropyl, R_{B}²=hydrogen and (R_{B}³)=5-Cl-2-OMe (S4-4) and
R_{B}¹=isopropyl, R_{B}²=hydrogen and (R_{B}³)=2-OMe (S4-5);
S4^{c}) Compounds from the class of the benzoylsulfamoylphenylureas of the formula (S4^{c}), as described in EP-A-365484,

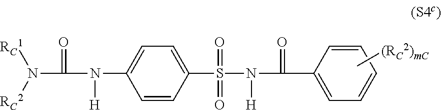

in which
R_{C}¹, R_{C}² are independently hydrogen, (C₁-C₅)-alkyl, (C₃-C₅)-cycloalkyl, (C₃-C₆)-alkenyl, (C₃-C₆)-alkynyl,
R_{C}³ is halogen, (C₁-C₄)-alkyl, (C₁-C₄)-alkoxy, CF₃ and
m_{C} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea;
S4^{d}) Compounds of the N-phenylsulfonylterephthalamide type of the formula (S4^{d}) and salts thereof, which are known, for example, from CN 101838227,

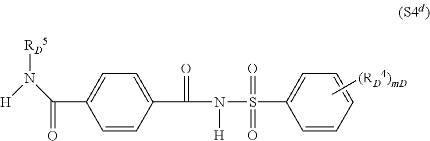

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.
S5) Active ingredients from the class of the hydroxyaromatics and the aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 2-hydroxycinnamic acid, 2,4-dichlorocinnamic acid, as described in WO-A-2004/084631, WO-A-2005/015994, WO-A-2005/016001.
S6) Active ingredients from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds from the class of the diphenylmethoxyacetic acid derivatives (S7), for example methyl diphenylmethoxyacetate (CAS Reg. No. 41858-19-9) (S7-1), ethyl diphenylmethoxyacetate or diphenylmethoxyacetic acid, as described in WO-A-98/38856.
S8) Compounds of the formula (S8), as described in WO-A-98/27049,

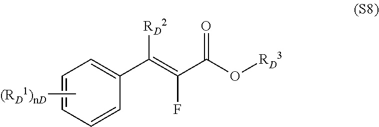

in which the symbols and indices are defined as follows:
R_{D}¹ is halogen, (C₁-C₄)-alkyl, (C₁-C₄)-haloalkyl, (C₁-C₄)-alkoxy, (C₁-C₄)-haloalkoxy,
R_{D}² is hydrogen or (C₁-C₄)-alkyl,
R_{D}³ is hydrogen, (C₁-C₅)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof,
n_{D} is an integer from 0 to 2.
S9) Active ingredients from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS reg. no.: 219479-18-2), 1,2-dihydro-4-hydroxy-1-methyl-3-(5-tetrazolylcarbonyl)-2-quinolone (CAS reg. no.: 95855-00-8), as described in WO-A-1999/000020.
S10) Compounds of the formulae (S10^{a}) or (S10^{b})
   as described in WO-A-2007/023719 and WO-A-2007/023764,

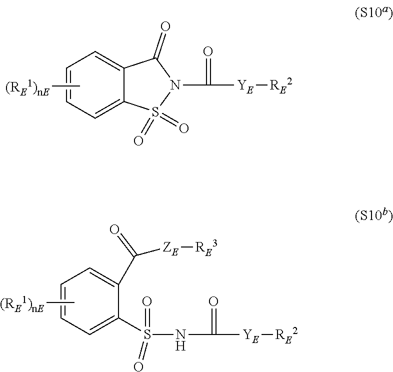

in which
R_{E}¹ is halogen, (C₁-C₄)-alkyl, methoxy, nitro, cyano, CF₃, OCF₃,
Y_{E}, Z_{E} are independently O or S,
n_{E} is an integer from 0 to 4,
R_{E}² is (C₁-C₁₆)-alkyl, (C₂-C₆)-alkenyl, (C₃-C₆)-cycloalkyl, aryl; benzyl, halobenzyl,
R_{E}³ is hydrogen or (C₁-C₆)-alkyl.
S11) Active ingredients of the oxyimino compounds type (S11), which are known as seed-dressing agents, for example
“oxabetrinil” ((Z)-1,3-dioxolan-2-ylmethoxyimino(phenyl)acetonitrile) (S11-1), which is known as a seed-dressing safener for millet/sorghum against metolachlor damage,
   “fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) Active ingredients from the class of the isothiochromanones (S12), for example methyl [(3-oxo-1H-2-benzothiopyran-4(3H)-ylidene)methoxy]acetate (CAS reg. no. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for maize against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8) (4-carboxy-3,4-dihydro-2H-1-benzopyran-4-acetic acid) (S13-4) from American Cyanamid, which is known as a safener for maize against damage by imidazolinones,
   “MG 191” (CAS Reg. No. 96420-72-3) (2-dichloromethyl-2-methyl-1,3-dioxolane) (S13-5) from Nitrokemia, which is known as a safener for maize,
   “MG 838” (CAS Reg. No. 133993-74-5) (2-propenyl 1-oxa-4-azaspiro[4.5]decane-4-carbodithioate) (S13-6) from Nitrokemia “disulfoton” (0,0-diethyl S-2-ethylthioethyl phosphorodithioate) (S13-7),
   “dietholate” (0,0-diethyl 0-phenyl phosphorothioate) (S13-8),
   “mephenate” (4-chlorophenyl methylcarbamate) (S13-9).
S14) Active ingredients which, in addition to herbicidal action against harmful plants, also have safener action on crop plants such as rice, for example
“dimepiperate” or “MY-93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as a safener for rice against damage by the herbicide imazosulfuron,
   “cumyluron”=“JC-940” (3-(2-chlorophenylmethyl)-1-(1-methyl-1-phenylethyl)urea, see JP-A-60087270), which is known as a safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

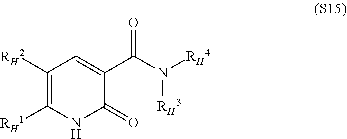

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ are independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxy, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted, or (C₃-C₆)-cycloalkyl, (C₄-C₆)-cycloalkenyl, (C₃-C₆)-cycloalkyl fused on one side of the ring to a 4- to 6-membered saturated or unsaturated carbocyclic ring, or (C₄-C₆)-cycloalkenyl fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active ingredients which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichloro-ethyl).

Preferred safeners in combination with the compounds of the general formula (I) according to the invention and/or salts thereof, in particular with the compounds of the formulae (1-1) to (1-209) or (2-1) to (2-14) and/or salts thereof, are: cloquintocet-mexyl, cyprosulfamide, fenchlorazole ethyl ester, isoxadifen-ethyl, mefenpyr-diethyl, fenclorim, cumyluron, 54-1 and S4-5, and particularly preferred safeners are: cloquintocet-mexyl, cyprosulfamide, isoxadifen-ethyl and mefenpyr-diethyl.

### Biological Examples

In the examples and tables below, the following abbreviations are used:

### Unwanted Plants/Weeds

ABUTH: Abutilon theophrasti
ALOMY: Alopecurus myosuroides
AMARE: Amaranthus retroflexus
DIGSA: Digitaria sanguinalis
ECHCG: Echinochloa crus-galli
KCHSC: Kochia scoparia
LOLRI: Lolium rigidum
MATIN: Matricaria inodora
POAAN: Poa annua
SETVI: Setaria viridis
STEME: Stellaria media
VERPE: Veronica persica
VIOTR: Viola tricolor
   A. Post-Emergence Herbicidal Efficacy at 1280 g/ha

Seeds of mono- and dicotyledonous weed plants were placed in plastic pots in sandy loam soil (doubly sown with in each case one species of mono- or dicotyledonous weed plants per pot), covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were applied to the green parts of the plants as aqueous suspension or emulsion with addition of 0.5% additive at a water application rate equivalent to 600 litres per hectare. After the test plants had been kept in the greenhouse under optimum growth conditions for about 3 weeks, the activity of the preparations was rated visually in comparison to untreated controls.

For example, 100% activity=the plants have died, 0% activity=like control plants.

Tables A1 to A12 below show the effects of selected compounds of the general formula (I) according to Table 1 on various harmful plants and at an application rate corresponding to 1280 g/ha, which were obtained by the trial procedure specified above.

TABLE A1
Post-emergence efficacy at 1280 g/ha against ABUTH in %

| Example number | Dosage [g/ha] | ABUTH |
|---|---|---|
| 1-1 | 1280 | 90 |
| 1-3 | 1280 | 90 |
| 1-23 | 1280 | 100 |
| 1-26 | 1280 | 100 |
| 1-27 | 1280 | 90 |
| 1-29 | 1280 | 90 |
| 1-31 | 1280 | 100 |
| 1-35 | 1280 | 100 |
| 1-37 | 1280 | 100 |
| 1-38 | 1280 | 100 |
| 1-40 | 1280 | 100 |
| 1-41 | 1280 | 90 |
| 1-42 | 1280 | 90 |
| 1-45 | 1280 | 90 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-59 | 1280 | 100 |
| 1-65 | 1280 | 100 |
| 1-66 | 1280 | 100 |
| 1-70 | 1280 | 100 |
| 1-81 | 1280 | 90 |
| 1-87 | 1280 | 100 |
| 1-89 | 1280 | 90 |
| 1-90 | 1280 | 90 |
| 1-91 | 1280 | 100 |
| 1-92 | 1280 | 90 |
| 1-93 | 1280 | 90 |
| 1-95 | 1280 | 100 |
| 1-96 | 1280 | 100 |
| 1-97 | 1280 | 100 |
| 1-99 | 1280 | 90 |
| 1-100 | 1280 | 90 |
| 1-101 | 1280 | 90 |
| 1-103 | 1280 | 100 |
| 1-104 | 1280 | 100 |
| 1-105 | 1280 | 90 |
| 1-106 | 1280 | 90 |
| 1-110 | 1280 | 90 |
| 1-111 | 1280 | 90 |
| 1-112 | 1280 | 100 |
| 1-127 | 1280 | 100 |
| 1-129 | 1280 | 100 |
| 1-130 | 1280 | 100 |
| 1-131 | 1280 | 100 |
| 1-133 | 1280 | 100 |
| 1-137 | 1280 | 90 |

TABLE A2
Post-emergence efficacy at 1280 g/ha against ALOMY in %

| Example number | Dosage [g/ha] | ALOMY |
|---|---|---|
| 1-35 | 1280 | 90 |
| 1-40 | 1280 | 100 |
| 1-54 | 1280 | 90 |
| 1-55 | 1280 | 90 |
| 1-66 | 1280 | 90 |
| 1-130 | 1280 | 90 |
| 1-133 | 1280 | 90 |

TABLE A3
Post-emergence efficacy at 1280 g/ha against AMARE in %

| Example number | Dosage [g/ha] | AMARE |
|---|---|---|
| 1-1 | 1280 | 100 |
| 1-3 | 1280 | 90 |
| 1-23 | 1280 | 100 |
| 1-26 | 1280 | 100 |
| 1-27 | 1280 | 100 |
| 1-29 | 1280 | 90 |
| 1-31 | 1280 | 90 |
| 1-32 | 1280 | 90 |
| 1-34 | 1280 | 90 |
| 1-35 | 1280 | 100 |
| 1-37 | 1280 | 100 |
| 1-38 | 1280 | 100 |
| 1-40 | 1280 | 100 |
| 1-41 | 1280 | 90 |
| 1-42 | 1280 | 100 |
| 1-45 | 1280 | 100 |
| 1-51 | 1280 | 100 |
| 1-52 | 1280 | 100 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-58 | 1280 | 90 |
| 1-59 | 1280 | 100 |
| 1-65 | 1280 | 100 |
| 1-66 | 1280 | 100 |
| 1-68 | 1280 | 100 |
| 1-69 | 1280 | 90 |
| 1-70 | 1280 | 90 |
| 1-71 | 1280 | 100 |
| 1-73 | 1280 | 90 |
| 1-74 | 1280 | 90 |
| 1-75 | 1280 | 90 |
| 1-76 | 1280 | 90 |
| 1-77 | 1280 | 90 |
| 1-78 | 1280 | 90 |
| 1-80 | 1280 | 100 |
| 1-81 | 1280 | 90 |
| 1-82 | 1280 | 100 |
| 1-87 | 1280 | 100 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-94 | 1280 | 90 |
| 1-95 | 1280 | 100 |
| 1-96 | 1280 | 100 |
| 1-97 | 1280 | 100 |
| 1-98 | 1280 | 100 |
| 1-99 | 1280 | 100 |
| 1-100 | 1280 | 100 |
| 1-101 | 1280 | 100 |
| 1-103 | 1280 | 100 |
| 1-104 | 1280 | 100 |
| 1-105 | 1280 | 90 |
| 1-106 | 1280 | 100 |
| 1-110 | 1280 | 100 |
| 1-111 | 1280 | 100 |
| 1-112 | 1280 | 100 |
| 1-119 | 1280 | 100 |
| 1-123 | 1280 | 100 |
| 1-127 | 1280 | 100 |
| 1-129 | 1280 | 100 |
| 1-130 | 1280 | 100 |
| 1-131 | 1280 | 100 |
| 1-133 | 1280 | 100 |
| 1-137 | 1280 | 100 |

TABLE A4
Post-emergence efficacy at 1280 g/ha against DIGSA in %

| Example number | Dosage [g/ha] | DIGSA |
|---|---|---|
| 1-1 | 1280 | 90 |
| 1-23 | 1280 | 100 |
| 1-26 | 1280 | 90 |
| 1-27 | 1280 | 90 |
| 1-29 | 1280 | 90 |
| 1-35 | 1280 | 90 |
| 1-37 | 1280 | 90 |
| 1-38 | 1280 | 90 |
| 1-40 | 1280 | 90 |
| 1-45 | 1280 | 90 |
| 1-53 | 1280 | 90 |
| 1-54 | 1280 | 90 |
| 1-55 | 1280 | 100 |
| 1-56 | 1280 | 90 |
| 1-57 | 1280 | 90 |
| 1-58 | 1280 | 90 |
| 1-59 | 1280 | 100 |
| 1-66 | 1280 | 100 |
| 1-90 | 1280 | 90 |
| 1-91 | 1280 | 90 |
| 1-92 | 1280 | 90 |
| 1-99 | 1280 | 90 |
| 1-104 | 1280 | 100 |
| 1-112 | 1280 | 90 |
| 1-131 | 1280 | 90 |
| 1-133 | 1280 | 100 |

TABLE A5
Post-emergence efficacy at 1280 g/ha against ECHCG in %

| Example number | Dosage [g/ha] | ECHCG |
|---|---|---|
| 1-23 | 1280 | 100 |
| 1-26 | 1280 | 90 |
| 1-31 | 1280 | 100 |
| 1-35 | 1280 | 100 |
| 1-37 | 1280 | 100 |
| 1-38 | 1280 | 100 |
| 1-40 | 1280 | 100 |
| 1-42 | 1280 | 90 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-58 | 1280 | 90 |
| 1-59 | 1280 | 90 |
| 1-65 | 1280 | 90 |
| 1-66 | 1280 | 100 |
| 1-81 | 1280 | 90 |
| 1-87 | 1280 | 90 |
| 1-96 | 1280 | 90 |
| 1-99 | 1280 | 100 |
| 1-101 | 1280 | 90 |
| 1-104 | 1280 | 100 |
| 1-106 | 1280 | 90 |
| 1-112 | 1280 | 90 |
| 1-130 | 1280 | 90 |
| 1-131 | 1280 | 100 |
| 1-133 | 1280 | 100 |

TABLE A6
Post-emergence efficacy at 1280 g/ha against KCHSC in %

| Example number | Dosage [g/ha] | KCHSC |
|---|---|---|
| 1-1 | 1280 | 90 |
| 1-3 | 1280 | 90 |
| 1-23 | 1280 | 90 |
| 1-26 | 1280 | 100 |
| 1-27 | 1280 | 90 |
| 1-29 | 1280 | 90 |
| 1-35 | 1280 | 100 |
| 1-37 | 1280 | 100 |
| 1-38 | 1280 | 100 |
| 1-39 | 1280 | 90 |
| 1-40 | 1280 | 100 |
| 1-45 | 1280 | 100 |
| 1-51 | 1280 | 90 |
| 1-52 | 1280 | 90 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-58 | 1280 | 90 |
| 1-59 | 1280 | 100 |
| 1-65 | 1280 | 100 |
| 1-66 | 1280 | 100 |
| 1-69 | 1280 | 90 |
| 1-71 | 1280 | 100 |
| 1-82 | 1280 | 90 |
| 1-87 | 1280 | 100 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-95 | 1280 | 100 |
| 1-96 | 1280 | 90 |
| 1-97 | 1280 | 100 |
| 1-98 | 1280 | 90 |
| 1-99 | 1280 | 100 |
| 1-100 | 1280 | 100 |
| 1-103 | 1280 | 90 |
| 1-104 | 1280 | 100 |
| 1-105 | 1280 | 90 |
| 1-106 | 1280 | 90 |
| 1-110 | 1280 | 90 |
| 1-111 | 1280 | 100 |
| 1-112 | 1280 | 100 |
| 1-119 | 1280 | 90 |
| 1-127 | 1280 | 100 |
| 1-129 | 1280 | 100 |
| 1-130 | 1280 | 100 |
| 1-131 | 1280 | 100 |
| 1-133 | 1280 | 100 |
| 1-137 | 1280 | 90 |

TABLE A7
Post-emergence efficacy at 1280 g/ha against LOLRI in %

| Example number | Dosage [g/ha] | LOLRI |
|---|---|---|
| 1-35 | 1280 | 90 |
| 1-40 | 1280 | 100 |
| 1-56 | 1280 | 90 |
| 1-66 | 1280 | 90 |
| 1-110 | 1280 | 90 |
| 1-133 | 1280 | 100 |

TABLE A8
Post-emergence efficacy at 1280 g/ha against MATIN in %

| Example number | Dosage [g/ha] | MATIN |
|---|---|---|
| 1-55 | 1280 | 90 |
| 1-59 | 1280 | 100 |
| 1-65 | 1280 | 100 |
| 1-70 | 1280 | 90 |
| 1-131 | 1280 | 90 |

TABLE A9
Post-emergence efficacy at 1280 g/ha against POAAN in %

| Example number | Dosage [g/ha] | POAAN |
|---|---|---|
| 1-1 | 1280 | 90 |
| 1-3 | 1280 | 100 |
| 1-23 | 1280 | 100 |
| 1-26 | 1280 | 90 |
| 1-27 | 1280 | 90 |
| 1-29 | 1280 | 90 |
| 1-31 | 1280 | 90 |
| 1-33 | 1280 | 90 |
| 1-35 | 1280 | 90 |
| 1-37 | 1280 | 100 |
| 1-38 | 1280 | 90 |
| 1-40 | 1280 | 90 |
| 1-41 | 1280 | 90 |
| 1-42 | 1280 | 90 |
| 1-45 | 1280 | 90 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-58 | 1280 | 90 |
| 1-59 | 1280 | 100 |
| 1-65 | 1280 | 100 |
| 1-66 | 1280 | 100 |
| 1-74 | 1280 | 90 |
| 1-77 | 1280 | 90 |
| 1-87 | 1280 | 100 |
| 1-90 | 1280 | 100 |
| 1-91 | 1280 | 100 |
| 1-92 | 1280 | 100 |
| 1-93 | 1280 | 100 |
| 1-94 | 1280 | 90 |
| 1-95 | 1280 | 100 |
| 1-96 | 1280 | 90 |
| 1-97 | 1280 | 100 |
| 1-98 | 1280 | 90 |
| 1-99 | 1280 | 100 |
| 1-100 | 1280 | 100 |
| 1-101 | 1280 | 100 |
| 1-103 | 1280 | 100 |
| 1-104 | 1280 | 100 |
| 1-105 | 1280 | 90 |
| 1-106 | 1280 | 100 |
| 1-110 | 1280 | 100 |
| 1-111 | 1280 | 100 |
| 1-112 | 1280 | 100 |
| 1-123 | 1280 | 90 |
| 1-127 | 1280 | 100 |
| 1-129 | 1280 | 100 |
| 1-130 | 1280 | 100 |
| 1-131 | 1280 | 100 |
| 1-133 | 1280 | 100 |
| 1-137 | 1280 | 90 |

TABLE A10
Post-emergence efficacy at 1280 g/ha against SETVI in %

| Example number | Dosage [g/ha] | SETVI |
|---|---|---|
| 1-23 | 1280 | 90 |
| 1-26 | 1280 | 90 |
| 1-27 | 1280 | 90 |
| 1-29 | 1280 | 90 |
| 1-31 | 1280 | 90 |
| 1-35 | 1280 | 90 |
| 1-38 | 1280 | 90 |
| 1-40 | 1280 | 90 |
| 1-45 | 1280 | 90 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-59 | 1280 | 90 |
| 1-65 | 1280 | 90 |
| 1-66 | 1280 | 100 |
| 1-74 | 1280 | 90 |
| 1-87 | 1280 | 90 |
| 1-91 | 1280 | 90 |
| 1-92 | 1280 | 100 |
| 1-95 | 1280 | 90 |
| 1-99 | 1280 | 90 |
| 1-100 | 1280 | 90 |
| 1-103 | 1280 | 90 |
| 1-104 | 1280 | 90 |
| 1-112 | 1280 | 90 |
| 1-127 | 1280 | 90 |
| 1-130 | 1280 | 90 |
| 1-131 | 1280 | 90 |
| 1-133 | 1280 | 100 |

TABLE A11
Post-emergence efficacy at 1280 g/ha against STEME in %

| Example number | Dosage [g/ha] | STEME |
|---|---|---|
| 1-1 | 1280 | 100 |
| 1-3 | 1280 | 90 |
| 1-8 | 1280 | 90 |
| 1-23 | 1280 | 90 |
| 1-26 | 1280 | 100 |
| 1-27 | 1280 | 90 |
| 1-31 | 1280 | 90 |
| 1-32 | 1280 | 90 |
| 1-33 | 1280 | 90 |
| 1-34 | 1280 | 90 |
| 1-35 | 1280 | 100 |
| 1-37 | 1280 | 90 |
| 1-38 | 1280 | 100 |
| 1-40 | 1280 | 100 |
| 1-42 | 1280 | 90 |
| 1-45 | 1280 | 90 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-58 | 1280 | 90 |
| 1-59 | 1280 | 100 |
| 1-65 | 1280 | 100 |
| 1-66 | 1280 | 100 |
| 1-69 | 1280 | 90 |
| 1-70 | 1280 | 90 |
| 1-71 | 1280 | 90 |
| 1-77 | 1280 | 90 |
| 1-80 | 1280 | 90 |
| 1-87 | 1280 | 90 |
| 1-90 | 1280 | 90 |
| 1-91 | 1280 | 100 |
| 1-92 | 1280 | 100 |
| 1-95 | 1280 | 100 |
| 1-96 | 1280 | 90 |
| 1-97 | 1280 | 90 |
| 1-98 | 1280 | 90 |
| 1-99 | 1280 | 100 |
| 1-100 | 1280 | 90 |
| 1-101 | 1280 | 90 |
| 1-103 | 1280 | 100 |
| 1-104 | 1280 | 100 |
| 1-105 | 1280 | 90 |
| 1-106 | 1280 | 100 |
| 1-110 | 1280 | 100 |
| 1-111 | 1280 | 100 |
| 1-112 | 1280 | 100 |
| 1-119 | 1280 | 100 |
| 1-123 | 1280 | 90 |
| 1-127 | 1280 | 100 |
| 1-129 | 1280 | 100 |
| 1-130 | 1280 | 100 |
| 1-131 | 1280 | 100 |
| 1-133 | 1280 | 100 |
| 1-137 | 1280 | 90 |

TABLE A12
Post-emergence efficacy at 1280 g/ha against VERPE in %

| Example number | Dosage [g/ha] | VERPE |
|---|---|---|
| 1-1 | 1280 | 100 |
| 1-3 | 1280 | 90 |
| 1-8 | 1280 | 100 |
| 1-23 | 1280 | 100 |
| 1-26 | 1280 | 100 |
| 1-27 | 1280 | 90 |
| 1-29 | 1280 | 100 |
| 1-30 | 1280 | 90 |
| 1-31 | 1280 | 90 |
| 1-32 | 1280 | 90 |
| 1-33 | 1280 | 90 |
| 1-34 | 1280 | 100 |
| 1-35 | 1280 | 100 |
| 1-36 | 1280 | 90 |
| 1-37 | 1280 | 100 |
| 1-38 | 1280 | 100 |
| 1-39 | 1280 | 100 |
| 1-40 | 1280 | 100 |
| 1-41 | 1280 | 90 |
| 1-42 | 1280 | 90 |
| 1-45 | 1280 | 100 |
| 1-51 | 1280 | 100 |
| 1-52 | 1280 | 90 |
| 1-53 | 1280 | 100 |
| 1-54 | 1280 | 100 |
| 1-55 | 1280 | 100 |
| 1-56 | 1280 | 90 |
| 1-57 | 1280 | 90 |
| 1-58 | 1280 | 90 |
| 1-59 | 1280 | 100 |
| 1-60 | 1280 | 100 |
| 1-65 | 1280 | 100 |
| 1-66 | 1280 | 100 |
| 1-69 | 1280 | 100 |
| 1-70 | 1280 | 90 |
| 1-71 | 1280 | 90 |
| 1-73 | 1280 | 90 |
| 1-75 | 1280 | 90 |
| 1-76 | 1280 | 90 |
| 1-77 | 1280 | 90 |
| 1-80 | 1280 | 90 |
| 1-81 | 1280 | 90 |
| 1-82 | 1280 | 90 |
| 1-87 | 1280 | 100 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-94 | 1280 | 100 |
| 1-95 | 1280 | 90 |
| 1-96 | 1280 | 100 |
| 1-97 | 1280 | 100 |
| 1-98 | 1280 | 100 |
| 1-99 | 1280 | 90 |
| 1-100 | 1280 | 100 |
| 1-101 | 1280 | 100 |
| 1-103 | 1280 | 100 |
| 1-104 | 1280 | 100 |
| 1-105 | 1280 | 90 |
| 1-106 | 1280 | 100 |
| 1-110 | 1280 | 100 |
| 1-111 | 1280 | 100 |
| 1-112 | 1280 | 100 |
| 1-119 | 1280 | 90 |
| 1-123 | 1280 | 100 |
| 1-127 | 1280 | 100 |
| 1-129 | 1280 | 90 |
| 1-130 | 1280 | 100 |
| 1-131 | 1280 | 100 |
| 1-133 | 1280 | 100 |
| 1-137 | 1280 | 90 |

The trial results show that inventive compounds of the general formula (I) in post-emergence treatment show good herbicidal efficacy against selected harmful plants, for example Abutilon theophrasti, Alopecurus myosuroides, Amaranthus retroflexus, Digitaria sanguinalis, Echinochloa crus-galli, Kochia scoparia, Lolium rigidum, Matricaria inodora, Poa annua, Setaria viridis, Stellaria media and Veronica persica, at a respective application rate of 1280 g of active substance per hectare.

B. Post-Emergence Herbicidal Efficacy at 80 g/ha

Seeds of monocotyledonous and dicotyledonous weeds and crop plants were placed in sandy loam in plastic or organic planting pots, covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were then sprayed onto the green parts of the plants as aqueous suspension or emulsion with addition of 0.5% additive at a water application rate of 600 l/ha (converted). After the test plants had been kept in the greenhouse under optimum growth conditions for about 3 weeks, the activity of the preparations was rated visually in comparison to untreated controls. For example, 100% activity=the plants have died, 0% activity=like control plants.

Tables B1 to B10 below show the effects of selected compounds of the general formula (I) according to Table 1 on various harmful plants and an application rate corresponding to 80 g/ha, which were obtained by the experimental procedure mentioned above.

TABLE B1
Post-emergence efficacy at 80 g/ha against ABUTH in %

| Example number | Dosage [g/ha] | ABUTH |
|---|---|---|
| 1-133 | 80 | 80 |
| 1-140 | 80 | 100 |

TABLE B2
Post-emergence efficacy at 80 g/ha against ALOMY in %

| Example number | Dosage [g/ha] | ALOMY |
|---|---|---|
| 1-140 | 80 | 80 |

TABLE B3
Post-emergence efficacy at 80 g/ha against AMARE in %

| Example number | Dosage [g/ha] | AMARE |
|---|---|---|
| 1-14 | 80 | 95 |
| 1-15 | 80 | 90 |
| 1-16 | 80 | 100 |
| 1-17 | 80 | 90 |
| 1-18 | 80 | 90 |
| 1-20 | 80 | 90 |
| 1-21 | 80 | 90 |
| 1-40 | 80 | 90 |
| 1-51 | 80 | 100 |
| 1-52 | 80 | 80 |
| 1-54 | 80 | 100 |
| 1-56 | 80 | 100 |
| 1-59 | 80 | 100 |
| 1-67 | 80 | 100 |
| 1-131 | 80 | 100 |
| 1-133 | 80 | 100 |
| 1-135 | 80 | 100 |
| 1-140 | 80 | 100 |
| 1-142 | 80 | 80 |
| 1-150 | 80 | 80 |
| 1-151 | 80 | 80 |
| 1-152 | 80 | 90 |
| 1-153 | 80 | 90 |
| 1-154 | 80 | 90 |
| 1-156 | 80 | 80 |
| 1-157 | 80 | 80 |
| 1-159 | 80 | 80 |
| 1-160 | 80 | 100 |
| 1-186 | 80 | 90 |
| 1-187 | 80 | 90 |
| 1-193 | 80 | 90 |
| 1-194 | 80 | 98 |

TABLE B4
Post-emergence efficacy at 80 g/ha against DIGSA in %

| Example number | Dosage [g/ha] | DIGSA |
|---|---|---|
| 1-140 | 80 | 90 |

TABLE B5
Post-emergence efficacy at 80 g/ha against ECHCG in %

| Example number | Dosage [g/ha] | ECHCG |
|---|---|---|
| 1-140 | 80 | 90 |

TABLE B6
Post-emergence efficacy at 80 g/ha against KCHSC in %

| Example number | Dosage [g/ha] | KCHSC |
|---|---|---|
| 1-40 | 80 | 90 |
| 1-56 | 80 | 80 |
| 1-59 | 80 | 90 |
| 1-133 | 80 | 100 |

TABLE B7
Post-emergence efficacy at 80 g/ha against POLCO in %

| Example number | Dosage [g/ha] | POLCO |
|---|---|---|
| 1-16 | 80 | 80 |
| 1-54 | 80 | 80 |
| 1-56 | 80 | 80 |
| 1-133 | 80 | 100 |
| 1-140 | 80 | 100 |

TABLE B8
Post-emergence efficacy at 80 g/ha against SETVI in %

| Example number | Dosage [g/ha] | SETVI |
|---|---|---|
| 1-140 | 80 | 80 |

TABLE B9
Post-emergence efficacy at 80 g/ha against VERPE in %

| Example number | Dosage [g/ha] | VERPE |
|---|---|---|
| 1-14 | 80 | 95 |
| 1-16 | 80 | 90 |
| 1-40 | 80 | 90 |
| 1-51 | 80 | 100 |
| 1-54 | 80 | 100 |
| 1-56 | 80 | 100 |
| 1-59 | 80 | 90 |
| 1-65 | 80 | 90 |
| 1-67 | 80 | 80 |
| 1-133 | 80 | 100 |
| 1-140 | 80 | 100 |
| 1-186 | 80 | 90 |
| 1-187 | 80 | 90 |
| 1-189 | 80 | 90 |
| 1-192 | 80 | 80 |
| 1-193 | 80 | 90 |

TABLE B10
Post-emergence efficacy at 80 g/ha against VIOTR in %

| Example number | Dosage [g/ha] | VIOTR |
|---|---|---|
| 1-40 | 80 | 100 |
| 1-54 | 80 | 100 |
| 1-56 | 80 | 100 |
| 1-59 | 80 | 100 |
| 1-65 | 80 | 100 |
| 1-67 | 80 | 100 |
| 1-131 | 80 | 90 |
| 1-133 | 80 | 100 |

The trial results show that inventive compounds of the general formula (I) in post-emergence treatment show good herbicidal efficacy against selected harmful plants, for example Abutilon theophrasti, Alopecurus myosuroides, Amaranthus retroflexus, Digitaria sanguinalis, Echinochloa crus-galli, Kochia scoparia, Polygonum convolvulus, Setaria viridis, Veronica persica and Viola tricolor, at a respective application rate of 80 g of active substance per hectare.

C. Pre-Emergence Herbicide Efficacy at 1280 g/ha

Seeds of mono- and dicotyledonous weed plants were placed in plastic pots in sandy loam soil (doubly sown with one species each of mono- or dicotyledonous weed plants per pot) and covered with soil. The compounds of the invention, formulated in the form of wettable powders (WP) or as emulsion concentrates (EC), were then applied to the surface of the covering soil as aqueous suspension or emulsion with addition of 0.5% additive at a water application rate of 600 litres per hectare (converted). After the treatment, the pots were placed in a greenhouse and kept under good growth conditions for the test plants. After about 3 weeks, the efficacy of the preparations was scored visually in comparison with untreated controls as percentages.

For example, 100% activity=the plants have died, 0% activity=like control plants.

Tables C₁ to C₁₂ below show the effects of selected compounds of the general formula (I) according to Table 1 on various harmful plants and at an application rate corresponding to 1280 g/ha, which were obtained by the trial procedure specified above.

TABLE C1
Pre-emergence efficacy at 1280 g/ha against ABUTH in %

| Example number | Dosage [g/ha] | ABUTH |
|---|---|---|
| 1-23 | 1280 | 100 |
| 1-26 | 1280 | 90 |
| 1-27 | 1280 | 100 |
| 1-29 | 1280 | 100 |
| 1-35 | 1280 | 90 |
| 1-39 | 1280 | 100 |
| 1-40 | 1280 | 100 |
| 1-42 | 1280 | 100 |
| 1-45 | 1280 | 100 |
| 1-51 | 1280 | 100 |
| 1-52 | 1280 | 90 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 90 |
| 1-59 | 1280 | 100 |
| 1-65 | 1280 | 100 |
| 1-66 | 1280 | 100 |
| 1-69 | 1280 | 100 |
| 1-70 | 1280 | 90 |
| 1-75 | 1280 | 90 |
| 1-76 | 1280 | 90 |
| 1-77 | 1280 | 90 |
| 1-80 | 1280 | 90 |
| 1-82 | 1280 | 100 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-95 | 1280 | 100 |
| 1-96 | 1280 | 100 |
| 1-98 | 1280 | 100 |
| 1-99 | 1280 | 90 |
| 1-100 | 1280 | 100 |
| 1-101 | 1280 | 90 |
| 1-103 | 1280 | 100 |
| 1-104 | 1280 | 100 |
| 1-110 | 1280 | 90 |
| 1-112 | 1280 | 90 |
| 1-129 | 1280 | 100 |
| 1-130 | 1280 | 100 |
| 1-131 | 1280 | 100 |
| 1-133 | 1280 | 100 |
| 2-6 | 1280 | 100 |

TABLE C2
Pre-emergence efficacy at 1280 g/ha against ALOMY in %

| Example number | Dosage [g/ha] | ALOMY |
|---|---|---|
| 1-40 | 1280 | 100 |
| 1-53 | 1280 | 100 |
| 1-54 | 1280 | 90 |
| 1-56 | 1280 | 100 |
| 1-57 | 1280 | 100 |
| 1-59 | 1280 | 100 |
| 1-65 | 1280 | 90 |
| 1-66 | 1280 | 90 |
| 1-133 | 1280 | 100 |

TABLE C3
Pre-emergence efficacy at 1280 g/ha against AMARE in %

| Example number | Dosage [g/ha] | AMARE |
|---|---|---|
| 1-1 | 1280 | 100 |
| 1-8 | 1280 | 100 |
| 1-23 | 1280 | 100 |
| 1-26 | 1280 | 100 |
| 1-27 | 1280 | 100 |
| 1-29 | 1280 | 100 |
| 1-31 | 1280 | 90 |
| 1-32 | 1280 | 90 |
| 1-33 | 1280 | 100 |
| 1-34 | 1280 | 90 |
| 1-35 | 1280 | 100 |
| 1-37 | 1280 | 100 |
| 1-38 | 1280 | 100 |
| 1-40 | 1280 | 100 |
| 1-42 | 1280 | 100 |
| 1-45 | 1280 | 100 |
| 1-51 | 1280 | 100 |
| 1-52 | 1280 | 100 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 90 |
| 1-59 | 1280 | 100 |
| 1-65 | 1280 | 100 |
| 1-66 | 1280 | 100 |
| 1-70 | 1280 | 100 |
| 1-71 | 1280 | 100 |
| 1-73 | 1280 | 90 |
| 1-74 | 1280 | 100 |
| 1-75 | 1280 | 100 |
| 1-76 | 1280 | 100 |
| 1-77 | 1280 | 100 |
| 1-80 | 1280 | 100 |
| 1-82 | 1280 | 100 |
| 1-87 | 1280 | 100 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-95 | 1280 | 100 |
| 1-96 | 1280 | 100 |
| 1-97 | 1280 | 100 |
| 1-98 | 1280 | 100 |
| 1-99 | 1280 | 100 |
| 1-100 | 1280 | 100 |
| 1-101 | 1280 | 100 |
| 1-103 | 1280 | 100 |
| 1-104 | 1280 | 100 |
| 1-105 | 1280 | 100 |
| 1-106 | 1280 | 90 |
| 1-110 | 1280 | 100 |
| 1-111 | 1280 | 100 |
| 1-112 | 1280 | 100 |
| 1-119 | 1280 | 90 |
| 1-123 | 1280 | 90 |
| 1-127 | 1280 | 100 |
| 1-129 | 1280 | 100 |
| 1-130 | 1280 | 100 |
| 1-131 | 1280 | 100 |
| 1-133 | 1280 | 100 |
| 1-137 | 1280 | 100 |
| 2-6 | 1280 | 90 |

TABLE C4
Pre-emergence efficacy at 1280 g/ha against DIGSA in %

| Example number | Dosage [g/ha] | DIGSA |
|---|---|---|
| 1-1 | 1280 | 100 |
| 1-8 | 1280 | 100 |
| 1-23 | 1280 | 100 |
| 1-26 | 1280 | 100 |
| 1-27 | 1280 | 100 |
| 1-29 | 1280 | 100 |
| 1-31 | 1280 | 100 |
| 1-35 | 1280 | 90 |
| 1-37 | 1280 | 100 |
| 1-38 | 1280 | 100 |
| 1-40 | 1280 | 100 |
| 1-42 | 1280 | 100 |
| 1-45 | 1280 | 100 |
| 1-51 | 1280 | 100 |
| 1-52 | 1280 | 100 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 100 |
| 1-59 | 1280 | 100 |
| 1-65 | 1280 | 100 |
| 1-66 | 1280 | 100 |
| 1-70 | 1280 | 100 |
| 1-74 | 1280 | 100 |
| 1-77 | 1280 | 90 |
| 1-87 | 1280 | 100 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-95 | 1280 | 90 |
| 1-96 | 1280 | 100 |
| 1-98 | 1280 | 100 |
| 1-99 | 1280 | 100 |
| 1-100 | 1280 | 100 |
| 1-101 | 1280 | 100 |
| 1-103 | 1280 | 90 |
| 1-104 | 1280 | 100 |
| 1-111 | 1280 | 90 |
| 1-112 | 1280 | 100 |
| 1-127 | 1280 | 90 |
| 1-129 | 1280 | 90 |
| 1-131 | 1280 | 100 |
| 1-133 | 1280 | 100 |
| 2-6 | 1280 | 90 |

TABLE C5
Pre-emergence efficacy at 1280 g/ha against ECHCG in %

| Example number | Dosage [g/ha] | ECHCG |
|---|---|---|
| 1-1 | 1280 | 90 |
| 1-8 | 1280 | 90 |
| 1-23 | 1280 | 100 |
| 1-26 | 1280 | 90 |
| 1-27 | 1280 | 90 |
| 1-29 | 1280 | 100 |
| 1-31 | 1280 | 100 |
| 1-35 | 1280 | 90 |
| 1-37 | 1280 | 90 |
| 1-38 | 1280 | 100 |
| 1-40 | 1280 | 100 |
| 1-42 | 1280 | 90 |
| 1-45 | 1280 | 100 |
| 1-51 | 1280 | 90 |
| 1-52 | 1280 | 90 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 90 |
| 1-59 | 1280 | 100 |
| 1-65 | 1280 | 100 |
| 1-66 | 1280 | 100 |
| 1-70 | 1280 | 90 |
| 1-87 | 1280 | 90 |
| 1-90 | 1280 | 100 |
| 1-91 | 1280 | 90 |
| 1-92 | 1280 | 100 |
| 1-93 | 1280 | 100 |
| 1-95 | 1280 | 90 |
| 1-96 | 1280 | 90 |
| 1-98 | 1280 | 90 |
| 1-99 | 1280 | 100 |
| 1-100 | 1280 | 100 |
| 1-101 | 1280 | 100 |
| 1-103 | 1280 | 90 |
| 1-104 | 1280 | 100 |
| 1-105 | 1280 | 90 |
| 1-112 | 1280 | 100 |
| 1-129 | 1280 | 100 |
| 1-131 | 1280 | 100 |
| 1-133 | 1280 | 100 |

TABLE C6
Pre-emergence efficacy at 1280 g/ha against KCHSC in %

| Example number | Dosage [g/ha] | KCHSC |
|---|---|---|
| 1-1 | 1280 | 90 |
| 1-23 | 1280 | 90 |
| 1-26 | 1280 | 100 |
| 1-27 | 1280 | 90 |
| 1-35 | 1280 | 100 |
| 1-40 | 1280 | 90 |
| 1-42 | 1280 | 100 |
| 1-45 | 1280 | 100 |
| 1-51 | 1280 | 100 |
| 1-52 | 1280 | 100 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-59 | 1280 | 100 |
| 1-65 | 1280 | 100 |
| 1-66 | 1280 | 100 |
| 1-70 | 1280 | 100 |
| 1-74 | 1280 | 90 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-95 | 1280 | 90 |
| 1-100 | 1280 | 100 |
| 1-112 | 1280 | 100 |
| 1-127 | 1280 | 90 |
| 1-129 | 1280 | 90 |
| 1-131 | 1280 | 100 |
| 1-133 | 1280 | 100 |

TABLE C7
Pre-emergence efficacy at 1280 g/ha against LOLRI in %

| Example number | Dosage [g/ha] | LOLRI |
|---|---|---|
| 1-29 | 1280 | 100 |
| 1-40 | 1280 | 100 |
| 1-45 | 1280 | 90 |
| 1-53 | 1280 | 100 |
| 1-54 | 1280 | 100 |
| 1-55 | 1280 | 90 |
| 1-56 | 1280 | 100 |
| 1-57 | 1280 | 100 |
| 1-59 | 1280 | 100 |
| 1-65 | 1280 | 100 |
| 1-133 | 1280 | 90 |

TABLE C8
Pre-emergence efficacy at 1280 g/ha against MATIN in %

| Example number | Dosage [g/ha] | MATIN |
|---|---|---|
| 1-29 | 1280 | 90 |
| 1-35 | 1280 | 90 |
| 1-40 | 1280 | 90 |
| 1-42 | 1280 | 90 |
| 1-45 | 1280 | 90 |
| 1-51 | 1280 | 100 |
| 1-52 | 1280 | 100 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 100 |
| 1-59 | 1280 | 100 |
| 1-65 | 1280 | 100 |
| 1-70 | 1280 | 100 |
| 1-75 | 1280 | 90 |
| 1-76 | 1280 | 90 |
| 1-89 | 1280 | 100 |
| 1-91 | 1280 | 100 |
| 1-92 | 1280 | 100 |
| 1-93 | 1280 | 100 |
| 1-100 | 1280 | 100 |
| 1-104 | 1280 | 100 |
| 1-129 | 1280 | 100 |
| 1-131 | 1280 | 100 |
| 1-133 | 1280 | 100 |

TABLE C9
Pre-emergence efficacy at 1280 g/ha against POAAN in %

| Example number | Dosage [g/ha] | POAAN |
|---|---|---|
| 1-1 | 1280 | 90 |
| 1-23 | 1280 | 90 |
| 1-26 | 1280 | 100 |
| 1-27 | 1280 | 90 |
| 1-29 | 1280 | 100 |
| 1-31 | 1280 | 100 |
| 1-33 | 1280 | 100 |
| 1-35 | 1280 | 90 |
| 1-37 | 1280 | 90 |
| 1-38 | 1280 | 100 |
| 1-40 | 1280 | 100 |
| 1-41 | 1280 | 90 |
| 1-42 | 1280 | 90 |
| 1-45 | 1280 | 90 |
| 1-51 | 1280 | 90 |
| 1-52 | 1280 | 100 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 100 |
| 1-59 | 1280 | 100 |
| 1-60 | 1280 | 90 |
| 1-65 | 1280 | 100 |
| 1-66 | 1280 | 100 |
| 1-70 | 1280 | 90 |
| 1-72 | 1280 | 90 |
| 1-74 | 1280 | 100 |
| 1-77 | 1280 | 90 |
| 1-78 | 1280 | 90 |
| 1-79 | 1280 | 90 |
| 1-80 | 1280 | 90 |
| 1-81 | 1280 | 100 |
| 1-82 | 1280 | 90 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-95 | 1280 | 90 |
| 1-98 | 1280 | 90 |
| 1-99 | 1280 | 100 |
| 1-100 | 1280 | 100 |
| 1-101 | 1280 | 100 |
| 1-103 | 1280 | 90 |
| 1-104 | 1280 | 100 |
| 1-105 | 1280 | 100 |
| 1-110 | 1280 | 90 |
| 1-112 | 1280 | 90 |
| 1-119 | 1280 | 90 |
| 1-127 | 1280 | 90 |
| 1-129 | 1280 | 90 |
| 1-130 | 1280 | 90 |
| 1-131 | 1280 | 90 |
| 1-133 | 1280 | 100 |
| 1-137 | 1280 | 90 |

TABLE C10
Pre-emergence efficacy at 1280 g/ha against SETVI in %

| Example number | Dosage [g/ha] | SETVI |
|---|---|---|
| 1-1 | 1280 | 90 |
| 1-8 | 1280 | 90 |
| 1-23 | 1280 | 100 |
| 1-26 | 1280 | 90 |
| 1-27 | 1280 | 100 |
| 1-29 | 1280 | 100 |
| 1-31 | 1280 | 100 |
| 1-33 | 1280 | 100 |
| 1-35 | 1280 | 90 |
| 1-37 | 1280 | 100 |
| 1-38 | 1280 | 100 |
| 1-40 | 1280 | 100 |
| 1-42 | 1280 | 100 |
| 1-45 | 1280 | 100 |
| 1-51 | 1280 | 100 |
| 1-52 | 1280 | 100 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 100 |
| 1-59 | 1280 | 100 |
| 1-65 | 1280 | 100 |
| 1-66 | 1280 | 100 |
| 1-70 | 1280 | 100 |
| 1-74 | 1280 | 100 |
| 1-87 | 1280 | 100 |
| 1-89 | 1280 | 90 |
| 1-90 | 1280 | 90 |
| 1-91 | 1280 | 100 |
| 1-92 | 1280 | 100 |
| 1-93 | 1280 | 100 |
| 1-96 | 1280 | 90 |
| 1-98 | 1280 | 90 |
| 1-99 | 1280 | 100 |
| 1-100 | 1280 | 100 |
| 1-101 | 1280 | 100 |
| 1-103 | 1280 | 100 |
| 1-104 | 1280 | 100 |
| 1-112 | 1280 | 90 |
| 1-127 | 1280 | 100 |
| 1-129 | 1280 | 90 |
| 1-130 | 1280 | 90 |
| 1-131 | 1280 | 100 |
| 1-133 | 1280 | 100 |
| 1-137 | 1280 | 100 |

TABLE C11
Pre-emergence efficacy at 1280 g/ha against STEME in %

| Example number | Dosage [g/ha] | STEME |
|---|---|---|
| 1-8 | 1280 | 90 |
| 1-23 | 1280 | 100 |
| 1-29 | 1280 | 90 |
| 1-33 | 1280 | 90 |
| 1-34 | 1280 | 90 |
| 1-35 | 1280 | 100 |
| 1-37 | 1280 | 90 |
| 1-38 | 1280 | 100 |
| 1-40 | 1280 | 100 |
| 1-42 | 1280 | 100 |
| 1-45 | 1280 | 100 |
| 1-51 | 1280 | 100 |
| 1-52 | 1280 | 100 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 100 |
| 1-59 | 1280 | 100 |
| 1-65 | 1280 | 100 |
| 1-66 | 1280 | 100 |
| 1-70 | 1280 | 100 |
| 1-71 | 1280 | 90 |
| 1-76 | 1280 | 90 |
| 1-77 | 1280 | 90 |
| 1-80 | 1280 | 100 |
| 1-82 | 1280 | 100 |
| 1-89 | 1280 | 90 |
| 1-90 | 1280 | 90 |
| 1-91 | 1280 | 100 |
| 1-92 | 1280 | 100 |
| 1-93 | 1280 | 100 |
| 1-95 | 1280 | 100 |
| 1-98 | 1280 | 90 |
| 1-99 | 1280 | 100 |
| 1-100 | 1280 | 100 |
| 1-101 | 1280 | 100 |
| 1-103 | 1280 | 100 |
| 1-104 | 1280 | 100 |
| 1-105 | 1280 | 90 |
| 1-110 | 1280 | 100 |
| 1-112 | 1280 | 90 |
| 1-127 | 1280 | 90 |
| 1-129 | 1280 | 100 |
| 1-131 | 1280 | 100 |
| 1-133 | 1280 | 100 |
| 1-137 | 1280 | 100 |
| 2-6 | 1280 | 100 |

TABLE C12
Pre-emergence efficacy at 1280 g/ha against VERPE in %

| Example number | Dosage [g/ha] | VERPE |
|---|---|---|
| 1-23 | 1280 | 100 |
| 1-27 | 1280 | 100 |
| 1-29 | 1280 | 100 |
| 1-33 | 1280 | 100 |
| 1-35 | 1280 | 100 |
| 1-38 | 1280 | 100 |
| 1-40 | 1280 | 100 |
| 1-42 | 1280 | 90 |
| 1-45 | 1280 | 100 |
| 1-51 | 1280 | 100 |
| 1-52 | 1280 | 90 |
| 1-53 | 1280 | 100 |
| 1-54 | 1280 | 100 |
| 1-55 | 1280 | 100 |
| 1-56 | 1280 | 100 |
| 1-57 | 1280 | 100 |
| 1-58 | 1280 | 90 |
| 1-59 | 1280 | 100 |
| 1-60 | 1280 | 90 |
| 1-65 | 1280 | 100 |
| 1-66 | 1280 | 100 |
| 1-70 | 1280 | 90 |
| 1-76 | 1280 | 90 |
| 1-89 | 1280 | 100 |
| 1-90 | 1280 | 100 |
| 1-91 | 1280 | 100 |
| 1-92 | 1280 | 100 |
| 1-93 | 1280 | 100 |
| 1-95 | 1280 | 100 |
| 1-98 | 1280 | 90 |
| 1-100 | 1280 | 100 |
| 1-103 | 1280 | 90 |
| 1-104 | 1280 | 100 |
| 1-105 | 1280 | 100 |
| 1-112 | 1280 | 100 |
| 1-127 | 1280 | 90 |
| 1-129 | 1280 | 90 |
| 1-130 | 1280 | 90 |
| 1-131 | 1280 | 100 |
| 1-133 | 1280 | 100 |

The trial results show that inventive compounds of the general formula (I) in pre-emergence treatment show good herbicidal efficacy against selected harmful plants, for example Abutilon theophrasti, Alopecurus myosuroides, Amaranthus retroflexus, Digitaria sanguinalis, Echinochloa crus-galli, Kochia scoparia, Lolium rigidum, Matricaria inodora, Poa annua, Setaria viridis, Stellaria media and Veronica persica, at a respective application rate of 1280 g of active substance per hectare.

D. Pre-Emergence Herbicide Efficacy at 80 g/ha

Seeds of monocotyledonous and dicotyledonous weed plants and crop plants were placed in plastic or organic planting pots and covered with soil. The compounds of the invention, formulated in the form of wettable powders (WP) or as emulsion concentrates (EC), were then applied to the surface of the covering soil as aqueous suspension or emulsion with addition of 0.5% additive at a water application rate equivalent to 600 l/ha. After the treatment, the pots were placed in a greenhouse and kept under good growth conditions for the test plants. After about 3 weeks, the efficacy of the preparations was scored visually in comparison with untreated controls as percentages. For example, 100% activity=the plants have died, 0% activity=like control plants.

Tables D1 to D12 below show the effects of selected compounds of the general formula (I) according to Table 1 on various harmful plants and an application rate corresponding to 80 g/ha, which were obtained by the experimental procedure mentioned above.

TABLE D1
Pre-emergence efficacy at 80 g/ha against ABUTH in %

| Example number | Dosage [g/ha] | ABUTH |
|---|---|---|
| 1-52 | 80 | 90 |
| 1-54 | 80 | 100 |
| 1-56 | 80 | 100 |
| 1-59 | 80 | 100 |
| 1-65 | 80 | 90 |
| 1-67 | 80 | 100 |
| 1-133 | 80 | 100 |
| 1-135 | 80 | 95 |
| 1-156 | 80 | 90 |
| 1-152 | 80 | 80 |
| 1-140 | 80 | 100 |

TABLE D2
Pre-emergence efficacy at 80 g/ha against ALOMY in %

| Example number | Dosage [g/ha] | ALOMY |
|---|---|---|
| 1-54 | 80 | 80 |
| 1-59 | 80 | 80 |
| 1-140 | 80 | 90 |

TABLE D3
Pre-emergence efficacy at 80 g/ha against AMARE in %

| Example number | Dosage [g/ha] | AMARE |
|---|---|---|
| 1-14 | 80 | 90 |
| 1-16 | 80 | 100 |
| 1-17 | 80 | 80 |
| 1-21 | 80 | 90 |
| 1-45 | 80 | 100 |
| 1-50 | 80 | 90 |
| 1-51 | 80 | 90 |
| 1-52 | 80 | 100 |
| 1-54 | 80 | 100 |
| 1-56 | 80 | 100 |
| 1-59 | 80 | 100 |
| 1-65 | 80 | 100 |
| 1-67 | 80 | 90 |
| 1-131 | 80 | 100 |
| 1-133 | 80 | 100 |
| 1-135 | 80 | 100 |
| 1-140 | 80 | 100 |
| 1-151 | 80 | 80 |
| 1-152 | 80 | 100 |
| 1-153 | 80 | 100 |
| 1-154 | 80 | 100 |
| 1-156 | 80 | 100 |
| 1-157 | 80 | 100 |
| 1-159 | 80 | 90 |
| 1-160 | 80 | 100 |
| 1-162 | 80 | 90 |
| 1-166 | 80 | 95 |
| 1-187 | 80 | 100 |
| 1-189 | 80 | 80 |
| 1-194 | 80 | 100 |

TABLE D4
Pre-emergence efficacy at 80 g/ha against DIGSA in %

| Example number | Dosage [g/ha] | DIGSA |
|---|---|---|
| 1-54 | 80 | 100 |
| 1-56 | 80 | 100 |
| 1-59 | 80 | 100 |
| 1-65 | 80 | 90 |
| 1-133 | 80 | 100 |
| 1-140 | 80 | 100 |
| 1-152 | 80 | 90 |
| 1-156 | 80 | 90 |
| 1-157 | 80 | 80 |
| 1-165 | 80 | 80 |
| 1-166 | 80 | 100 |
| 1-194 | 80 | 90 |

TABLE D5
Pre-emergence efficacy at 80 g/ha against ECHCG in %

| Example number | Dosage [g/ha] | ECHCG |
|---|---|---|
| 1-54 | 80 | 100 |
| 1-56 | 80 | 80 |
| 1-59 | 80 | 100 |
| 1-133 | 80 | 80 |
| 1-140 | 80 | 100 |

TABLE D6
Pre-emergence efficacy at 80 g/ha against KCHSC in %

| Example number | Dosage [g/ha] | KCHSC |
|---|---|---|
| 1-50 | 80 | 90 |
| 1-54 | 80 | 100 |
| 1-56 | 80 | 90 |
| 1-59 | 80 | 100 |
| 1-65 | 80 | 100 |
| 1-133 | 80 | 100 |

TABLE D7
Pre-emergence efficacy at 80 g/ha against LOLRI in %

| Example number | Dosage [g/ha] | LOLRI |
|---|---|---|
| 1-140 | 80 | 90 |

TABLE D8
Pre-emergence efficacy at 80 g/ha against MATIN in %

| Example number | Dosage [g/ha] | MATIN |
|---|---|---|
| 1-54 | 80 | 90 |
| 1-56 | 80 | 100 |
| 1-59 | 80 | 100 |
| 1-65 | 80 | 100 |
| 1-133 | 80 | 80 |
| 1-140 | 80 | 80 |
| 1-156 | 80 | 80 |

TABLE D9
Pre-emergence efficacy at 80 g/ha against POLCO in %

| Example number | Dosage [g/ha] | POLCO |
|---|---|---|
| 1-54 | 80 | 80 |
| 1-56 | 80 | 90 |
| 1-59 | 80 | 90 |
| 1-131 | 80 | 80 |
| 1-133 | 80 | 80 |
| 1-135 | 80 | 90 |
| 1-140 | 80 | 90 |
| 1-152 | 80 | 90 |
| 1-154 | 80 | 80 |
| 1-156 | 80 | 80 |
| 1-162 | 80 | 80 |
| 1-165 | 80 | 80 |
| 1-166 | 80 | 90 |
| 1-194 | 80 | 90 |

TABLE D10
Pre-emergence efficacy at 80 g/ha against SETVI in %

| Example number | Dosage [g/ha] | SETVI |
|---|---|---|
| 1-54 | 80 | 100 |
| 1-56 | 80 | 100 |
| 1-59 | 80 | 100 |
| 1-65 | 80 | 90 |
| 1-133 | 80 | 100 |
| 1-140 | 80 | 100 |
| 1-152 | 80 | 80 |
| 1-166 | 80 | 80 |
| 1-194 | 80 | 100 |

TABLE D11
Pre-emergence efficacy at 80 g/ha against VERPE in %

| Example number | Dosage [g/ha] | VERPE |
|---|---|---|
| 1-16 | 80 | 90 |
| 1-51 | 80 | 80 |
| 1-54 | 80 | 90 |
| 1-56 | 80 | 90 |
| 1-59 | 80 | 90 |
| 1-65 | 80 | 100 |
| 1-133 | 80 | 90 |
| 1-135 | 80 | 95 |
| 1-140 | 80 | 100 |
| 1-194 | 80 | 90 |

TABLE D12
Pre-emergence efficacy at 80 g/ha against VIOTR in %

| Example number | Dosage [g/ha] | VIOTR |
|---|---|---|
| 1-40 | 80 | 100 |
| 1-54 | 80 | 100 |
| 1-56 | 80 | 100 |
| 1-59 | 80 | 100 |
| 1-65 | 80 | 100 |
| 1-131 | 80 | 90 |
| 1-133 | 80 | 80 |

The trial results show that inventive compounds of the general formula (I) in pre-emergence treatment show good herbicidal efficacy against selected harmful plants, for example Abutilon theophrasti, Alopecurus myosuroides, Amaranthus retroflexus, Digitaria sanguinalis, Echinochloa crus-galli, Kochia scoparia, Lolium rigidum, Matricaria inodora, Polygonum convolvulus, Setaria viridis, Veronica persica and Viola tricolor, at a respective application rate of 80 g of active substance per hectare.

1. Substituted 2-C-azines of the general formula (I) or salts thereof:
in which 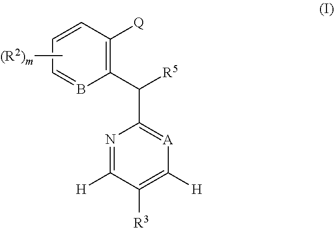
A is nitrogen or CR¹,
R¹ is hydrogen, CN or halogen,
B is CH, CR² or N,
Q is Y—(C₂-C₆)-haloalkyl, where Y denotes a direct bond, oxygen, S(O)ₙ, CO or OSO₂, or
Q is Z-aryl or Z-heteroaryl, where aryl is substituted by 1 to 5 substituents independently selected from the group of R⁴, where heteroaryl is substituted by up to 2 substituents independently selected from the group of R⁴, and where Z denotes a direct bond, O, S(O)ₙ or CH₂,
R² is independently halogen, cyano, nitro, amino, (C₁-C₄)-alkyl, (C₁-C₄)-haloalkyl, cyclopropyl, (C₁-C₄)-alkoxy, (C₁-C₄)-haloalkoxy or (C₁-C₄)-alkyl-S(O)ₙ—,
m is 0, 1, 2 or 3,
n is 0, 1 or 2,
R³ is hydrogen, halogen, cyano, nitro, (C₁-C₄)-alkyl, (C₁-C₄)-haloalkyl, (C₁-C₄)-alkoxy, (C₁-C₂)-haloalkoxy or (C₁-C₄)-alkyl-S(O)ₙ—,
under the condition that, when A is CR¹, R³ must not be hydrogen,
R⁴ is halogen, cyano, nitro, (C₁-C₄)-alkyl, (C₁-C₅)-haloalkyl, (C₁-C₄)-alkoxy, (C₁-C₂)-haloalkoxy, (C₁-C₄)-alkoxymethyl or (C₁-C₄)-alkyl-S(O)ₙ—,
and
R⁵ is hydrogen or CN.

2. Compounds of the general formula (I) according to claim 1 or salts thereof, in which
A is nitrogen or CR¹,
R¹ is hydrogen, CN or halogen,
B is CH, CR² or N,
Q is Y—(C₃-C₅)-haloalkyl, where Y denotes a direct bond, oxygen, S(O)ₙ, CO or OSO₂, or
Q is Z-aryl or Z-heteroaryl, where aryl is substituted by 1 to 3 substituents independently selected from the group of R⁴, where heteroaryl is substituted by up to 2 substituents independently selected from the group of R⁴, and where Z denotes a direct bond, O, S(O)ₙ or CH₂,
R² is independently halogen, cyano, nitro, amino, (C₁-C₄)-alkyl, (C₁-C₄)-haloalkyl, cyclopropyl, (C₁-C₄)-alkoxy, (C₁-C₄)-haloalkoxy or (C₁-C₄)-alkyl-S(O)ₙ—,
m is 0, 1, 2 or 3,
n is 0, 1 or 2,
R³ is hydrogen, halogen, cyano, (C₁-C₄)-alkyl, (C₁-C₄)-haloalkyl, (C₁-C₄)-alkoxy or (C₁-C₂)-haloalkoxy,
under the condition that, when A is CR¹, R³ must not be hydrogen,
R⁴ is halogen, cyano, (C₁-C₄)-alkyl, (C₁-C₅)-haloalkyl or (C₁-C₄)-alkoxymethyl,
and
R⁵ is hydrogen or CN.

3. Compounds of the general formula (I) according to claim 1 or salts thereof, in which
A is nitrogen or CR¹,
R¹ is hydrogen, cyano, fluorine or chlorine,
B is CH, CR² or N,
Q is Y—(C₃-C₅)-haloalkyl, where Y denotes a direct bond, oxygen, S, CO or OSO₂, or
Q is Z-aryl or Z-heteroaryl, where aryl is substituted by 1 or 2 substituents independently selected from the group of R⁴, where heteroaryl is substituted by up to 2 substituents independently selected from the group of R⁴, and where Z denotes a direct bond, O, S or CH₂,
R² is independently fluorine, chlorine, bromine, cyano, methyl, CF₃ or methoxy,
m is 0, 1 or 2,
R³ is hydrogen, chlorine, bromine, fluorine, cyano, methyl, CF₃, methoxy or CHF₂O, under the condition that, when A is CR¹, R³ must not be hydrogen,
R⁴ is fluorine, chlorine, methyl, CHF₂, CF₃, methoxymethyl,
and
R⁵ is hydrogen or CN.

4. Compounds of the general formula (I) according to claim 1 or salts thereof, in which
A is nitrogen or CR¹,
R¹ is hydrogen, cyano, fluorine or chlorine,
B is CH, CR² or N,
Q is Y—(C₃-C₅)-haloalkyl, where Y denotes a direct bond, oxygen, S, CO or OSO₂, or
Q is Z-aryl or Z-heteroaryl, where aryl is substituted by 1 or 2 substituents independently selected from the group of R⁴, where heteroaryl is substituted by up to 2 substituents independently selected from the group of R⁴, and where Z denotes a direct bond, O, S or CH₂,
R² is independently fluorine, chlorine, bromine, cyano, methyl, CF₃ or methoxy,
m is 0, 1 or 2,
R³ is hydrogen, chlorine, bromine, fluorine, cyano, methyl, CF₃, methoxy or CHF₂O, under the condition that, when A is CR¹, R³ must not be hydrogen,
R⁴ is fluorine, chlorine, methyl, CHF₂, CF₃ or methoxymethyl,
and
R⁵ is hydrogen or CN.

5. Compounds of the general formula (I) according to claim 1 or salts thereof, in which
A is nitrogen, CH, CCN or CF,
B is CH,
Q is (CH₂)₃CF₃, (CH₂)₄CF₃, S(CH₂)₃CF₃, S(CH₂)₃Cl, SO(CH₂)₃Cl, SO(CH₂)₂CF₃, SO(CH₂)₃CF₃, SO₂(CH₂)₃CF₃, O(CH₂)₃CF₃, C(O)(CH₂)₃CF₃, OSO₂(CH₂)₃CF₃, OSO₂(CH₂)₂CF₃, OSO₂(CH₂)₃Cl, 4-fluorobenzyl, 4-chlorophenyl, 3,4-difluorophenyl, 4-fluorophenoxy, 4-fluorophenylsulfanyl, 2,4-Cl₂-phenylsulfonyl, 2,4-C₁₂-phenylsulfanyl, 2,4-C₁₂-phenylsulfinyl, 4-(CF₃)-pyrazol-1-yl, 5-Cl-pyrimidin-2-oxy, 5-F-pyrimidin-2-oxy, 5-Cl-3-F-pyridin-2-oxy, 3-(CHF₂)-isoxazol-5-yl, 5-(CHF₂)-isoxazol-3-yl, 1,2,4-oxadiazol-3-yl, 5-methyl-1,2,4-oxadiazol-3-yl, 5-(CHF₂)-1,2,4-oxadiazol-3-yl, 5-(CF₃)-1,2,4-oxadiazol-3-yl, 5-F-pyrimidin-2-ylsulfanyl, 5-Cl-pyrimidin-2-ylsulfanyl, 5-Cl-pyrimidin-2-ylsulfinyl, 5-Cl-pyrimidin-2-ylsulfonyl, 4-Cl-pyrazol-1-ylmethyl, 4-Br-pyrazol-1-ylmethyl or 4-(CF₃)-pyrazol-1-ylmethyl,
R² is independently fluorine, chlorine, bromine, cyano, methyl, CF₃ or methoxy,
m is 0, 1 or 2,
R³ is chlorine, bromine or fluorine
and
R⁵ is hydrogen or CN.

6. An herbicidal composition, characterized by a herbicidally active amount of at least one compound of the general formula (I) according to claim 1.

7. An herbicidal composition according to claim 6 in a mixture with formulation auxiliaries.

8. An herbicidal composition according to claim 6, comprising at least one further pesticidally active substance from the group of insecticides, acaricides, herbicides, fungicides, safeners and growth regulators.

9. An herbicidal composition according to claim 8, comprising a safener.

10. An herbicidal composition according to claim 9, comprising cyprosulfamide, cloquintocet-mexyl, mefenpyr-diethyl or isoxadifen-ethyl.

11. An herbicidal composition according to claim 6, comprising a further herbicide.

12. A method of controlling unwanted plants, characterized in that an effective amount of at least one compound of the general formula (I) according to claim 1 is applied to the plants or to the site of the unwanted vegetation.

13. A method of using compounds of the general formula (I) according to claim 1 for controlling unwanted plants.

14. A method according to claim 13, characterized in that the compounds of the general formula (I) are used for controlling unwanted plants in crops of useful plants.

15. A method according to claim 14, characterized in that the useful plants are transgenic useful plants.
